(19) United States
(12) Patent Application Publication
(10) Pub. No.: US 2024/0209050 A1
(43) Pub. Date: Jun. 27, 2024

(54) MUTANT IL-15 COMPOSITIONS AND METHODS THEREOF

(71) Applicant: LEGEND BIOTECH IRELAND LIMITED, Dublin 18 (IE)

(72) Inventors: Ming ZENG, Nanjing (CN); Huihui ZHANG, Nanjing (CN); Zhigang LI, Nanjing (CN); Xiaohu FAN, Nanjing (CN); Shuai YANG, Nanjing (CN); Xiaojie TU, Nanjing (CN); Shu WU, Nanjing (CN)

(21) Appl. No.: 18/576,630

(22) PCT Filed: Jul. 8, 2022
(86) PCT No.: PCT/CN2022/104638

§ 371 Date: Jan. 4, 2024

(30) Foreign Application Priority Data
Jul. 9, 2021 (WO) PCT/CN2021/105481
Jul. 9, 2021 (WO) PCT/CN2021/105484

(51) Int. Cl. C07K 14/54; A61K 39/00; A61P 35/00; C07K 14/715; C12N 5/0783; C12N 15/86

(52) U.S. Cl. CPC C07K 14/5443; A61K 39/4611; A61K 39/4613; A61K 39/4631; A61P 35/00; C07K 14/7155; C12N 5/0636; C12N 5/0646; C12N 15/86; A61K 2239/15; A61K 2239/21; C07K 2319/03; C07K 2319/035; C12N 2510/00

(57) ABSTRACT

The present application provides modified immune cells expressing a mutant IL-15 polypeptide. In some embodiments, the modified immune cell further comprises an engineered receptor such as a chimeric antigen receptor (CAR). The present application also provides methods and pharmaceutical compositions for cancer treatment using the modified immune cells described herein.

### CROSS REFERENCE TO RELATED APPLICATION

This application claims priority benefits of International Application Nos. PCT/CN2021/105481 and PCT/CN2021/105484, filed on Jul. 9, 2021, the contents of which are incorporated herein by reference in their entirety.

### SEQUENCE STATEMENT

The content of the following submission on ASCII text file is incorporated herein by reference in its entirety: a computer readable form (CRF) of the Sequence Listing (file name: P11200-PCT.220708.Sequence listing.xml, date recorded: Jul. 8, 2022, size: 102, 176 bytes).

### FIELD

The present application relates to modified immune cells that express an IL-15 polypeptide, and methods of use thereof for treating a disease or condition such as cancer.

### BACKGROUND

Chimeric antigen receptor (CAR) T cells are cells that have been modified to produce an engineered T cell receptor in order to elicit an immune response. For example, CAR-T cells may be designed to more effectively recognize cancer cells for improved cancer therapy. An alternative approach to CAR-T cell treatment is the use of natural killer (NK) cells, which are immune cells that kill target cells (e.g., tumor cells) via spontaneous cytotoxic activity independent of tumor antigen. CAR-NK cells could therefore be engineered to target diverse antigens, increase targeting of solid tumors, and overall achieve an effective anti-tumor response. Despite the success of CAR-NK cell therapies, these methods often suffer from higher rates of cytokine production (e.g., cytokine storm) that may cause damage at the site of injury. There remains a need for highly efficient cell-based cancer immunotherapy.

Interleukin 15 (IL-15) is a cytokine that plays a role in the development and control of the immune system. In particular, IL-15 induces the proliferation, function, and development of CD8+ T cells, Natural Killer (NK) cells, killer T cells, B cells, intestinal intraepithelial lymphocytes (IEL) and antigen-presenting cells (APC). Studies have shown that IL-15 is a potent activator of pro-inflammatory eukaryotic cell signaling. IL-15 stimulates the production of pro-inflammatory cytokines and chemokines in a number of innate and non-immune cells, including dendritic cells (DCs), NK cells, epithelial cells, and lymph node stromal cells. IL-15 acts on cells in both lymphoid and non-lymphoid compartments (Van Belle and Grooten, Arch Immunol Ther Exp (2005) 53:115). Given its crucial role in the immune system, IL-15 administration has been employed to strengthen immune responses. Conversely, inhibitors of IL-15 activity can diminish autoimmune and other undesirable immune responses (Waldmann, T A, 2006, Nature Rev. Immunol. 6:595-601). Engineered immune cells, such as T cells and NK cells, expressing CARs can be armored with IL-15 in order to provide enhanced anti-tumor activity. See, for example U.S. Pat. Nos. 9,629,877 and 10,428,305.

The disclosures of all publications, patents, patent applications and published patent applications referred to herein are hereby incorporated herein by reference in their entirety.

### BRIEF SUMMARY

The present application provides modified immune cells that express a mutant IL-15 polypeptide, and methods of use thereof for treating a disease or condition, such as cancer.

One aspect of the present application provides a modified immune cell comprising a first heterologous nucleic acid sequence encoding an IL-15 polypeptide comprising one or more amino acid substitutions at positions 8 and/or 62, wherein numbering of the amino acid residue positions is according to SEQ ID NO: 1. In some embodiments, the IL-15 polypeptide comprises an amino acid substitution at position 62. In some embodiments, the IL-15 polypeptide comprises an amino acid residue selected from the group consisting of Glycine (G), Isoleucine (I), Glutamine (Q), Valine (V), Proline (P), Leucine (L), Alanine (A), Serine (S) and Tyrosine (Y) at position 62. In some embodiments, the amino acid substitution at position 62 is selected from the group consisting of T62G, T62I, T62Q, T62V, T62P, T62L, T62A, T62S and T62Y. In some embodiments, the amino acid substitution at position 62 is T62G. In some embodiments, the IL-15 polypeptide comprises an amino acid sequence having at least 90% sequence identity to the amino acid sequence of SEQ ID NO: 7. In some embodiments, the IL-15 polypepitde comprises SEQ ID NO: 7.

In some embodiments according to any one of the modified immune cells described above, the IL-15 polypeptide comprises an amino acid substitution at position 8. In some embodiments, the IL-15 polypeptide comprises an amino acid residue Glutamic acid (E) at position 8. In some embodiments, the amino acid substitution at position 8 is D8E. In some embodiments, the IL-15 polypeptide comprises the amino acid sequence having at least 90% sequence identity to the amino acid sequence of SEQ ID NO: 5. In some embodiments, the IL-15 polypepitde comprises SEQ ID NO: 5.

One aspect of the present application provides a modified immune cell comprising a first heterologous nucleic acid sequence encoding an IL-15 polypeptide comprising one or more amino acid substitutions at positions 3 and/or 25, wherein numbering of the amino acid residue positions is according to SEQ ID NO: 1. In some embodiments, the IL-15 polypeptide comprises an amino acid substitution at position 3. In some embodiments, the IL-15 polypeptide comprises an amino acid residue Tyrosine (Y) at position 3. In some embodiments, the amino acid substitution at position 3 is V3Y. In some embodiments, the IL-15 polypeptide comprises an amino acid sequence having at least 90% sequence identity to the amino acid sequence of SEQ ID NO: 78. In some embodiments, the IL-15 polypepitde comprises SEQ ID NO: 78.

In some embodiments according to any one of the modified immune cells described above, the IL-15 polypeptide comprises an amino acid substitution at position 25. In some embodiments, the IL-15 polypeptide comprises an amino acid residue Phenylalanine (F) at position 25. In some embodiments, the amino acid substitution at position 25 is L25F. In some embodiments, the IL-15 polypeptide comprises the amino acid sequence having at least 90% sequence identity to the amino acid sequence of SEQ ID NO: 79. In some embodiments, the IL-15 polypepitde comprises SEQ ID NO: 79.

One aspect of the present application provides a modified immune cell comprising a first heterologous nucleic acid sequence encoding an TL-15 polypeptide that induces secretion of an inflammatory cytokine by the modified immune cell at a level that is least 50% lower than that by a modified immune cell comprising a heterologous nucleic acid sequence encoding a wildtype IL-15 polypeptide. In some embodiments, the inflammatory cytokine is IFNγ, TNFα, and/or GM-CSF. In some embodiments, the present application provides a modified immune cell comprising a first heterologous nucleic acid sequence encoding an IL-15 polypeptide that enhances anti-tumor activity of the modified immune cell.

In some embodiments according to any one of the modified immune cells described above, the one or more amino acid substitutions reduce affinity of the IL-15 polypeptide to IL-15Rβ compared to an IL-15 polypeptide that does not comprise the one or more amino acid substitutions (e.g., a wildtype IL-15 polypeptide).

In some embodiments according to any one of the modified immune cells described above, the IL-15 polypeptide is secreted.

In some embodiments according to any one of the modified immune cells described above, the IL-15 polypeptide is membrane bound. In some embodiments, the IL-15 polypeptide is bound to the membrane via a glycosylphosphatidylinositol (GPI)—anchoring peptide sequence. In some embodiments, the GPI-anchoring peptide sequence is attached to a GPI linker. In some embodiments, the IL-15 polypeptide is bound to the membrane via a transmembrane domain. In some embodiments, the IL-15 polypeptide is bound to the membrane via a membrane anchoring domain.

In some embodiments according to any one of the modified immune cells described above, the IL-15 polypeptide is a fusion protein comprising an IL-15 fragment fused to a second polypeptide fragment. In some embodiments, the second polypeptide fragment is selected from the group consisting of IL-15Rα, an extracellular domain of IL-15Rα, a Sushi domain of IL-15Rα, a transmembrane domain of IL-15Rα, IL-15Rβ, common gamma chain (γc), and combinations thereof. In some embodiments, the second polypeptide fragment comprises an amino acid sequence selected from the group consisting of SEQ ID NOs: 50-55. In some embodiments according to any one of the modified immune cells described above, the IL-15 polypeptide comprises: (a) an antigen-binding domain; (b) an IL-15 fragment; (c) a transmembrane domain; and (d) an intracellular domain.

In some embodiments according to any one of the modified immune cells described above, the modified immune cell comprises a second heterologous nucleic acid sequence encoding an engineered receptor. In some embodiments, the engineered receptor is a chimeric antigen receptor (CAR). In some embodiments, the CAR is a BCMA CAR, a CD19 CAR, or a GPC3 CAR. In some embodiments, the engineered receptor is a modified T-cell receptor (TCR). In some embodiments, the engineered receptor is a T-cell antigen coupler (TAC) receptor. In some embodiments, the first nucleic acid sequence and the second nucleic acid sequence are operably linked to the same promoter. In some embodiments the first nucleic acid and the second nucleic acid are operably linked to separate promoters.

In some embodiments according to any one of the modified immune cells described above, the modified immune cell is selected from the group consisting of a cytotoxic T cell, a helper T cell, a natural killer (NK) cell, an NK-cell, an iNK-T cell, an NK-T like cell, an aβT cell and a γδT cell. In some embodiments, the modified immune cell is an NK cell. In some embodiments, the modified immune cell is a cytotoxic T cell.

In some embodiments according to any one of the modified immune cells described above, the modified immune cell has reduced toxicity in vivo when administered to an individual compared to a modified immune cell that comprises a heterologous nucleic acid encoding a wildtype IL-15 polypeptide. In some embodiments, the modified immune cell has improved safety in vivo when administered to an individual compared to a modified immune cell that comprises a heterologous nucleic acid encoding a wildtype IL-15 polypeptide. In some embodiments, the modified immune cell has improved anti-tumor activity compared to a modified immune cell that comprises a heterologous nucleic acid encoding a wildtype IL-15 polypeptide.

One aspect of the present application provides a method of producing a modified immune cell, comprising: introducing into a precursor immune cell a first nucleic acid sequence encoding an IL-15 polypeptide comprising one or more amino acid substitutions at positions 8 and/or 62, wherein numbering of the amino acid residue positions is according to SEQ ID NO: 1.

One aspect of the present application provides a method of producing a modified immune cell, comprising: introducing into a precursor immune cell a first nucleic acid sequence encoding an IL-15 polypeptide comprising one or more amino acid substitutions at positions 3 and/or 25, wherein numbering of the amino acid residue positions is according to SEQ ID NO: 1.

In some embodiments according to any one of the methods of production described above, the precursor immune cell is selected from the group consisting of a cytotoxic T cell, a helper T cell, an NK cell, an NK-T cell, an iNK-T cell, an NK-T like cell, an aβT cell and a γδT cell. In some embodiments, the precursor immune cell comprises an engineered receptor. In some embodiments according to any one of the methods of production described above, the method further comprises introducing into the precursor immune cell a second nucleic acid encoding an engineered receptor. In some embodiments, the engineered receptor is a chimeric antigen receptor (CAR), a modified T-cell receptor (TCR), or a T-cell antigen coupler (TAC) receptor.

In some embodiments according to any one of the methods of production described above, the first nucleic acid sequence and the second nucleic acid sequence are on the same vector. In some embodiments, the vector is a viral vector. In some embodiments, the viral vector is selected from the group consisting of an adenoviral vector, an adeno-associated virus vector, a retroviral vector, a lentiviral vector, a herpes simplex viral vector, and derivatives thereof.

In some embodiments according to any one of the methods of production described above, the method further comprises isolating or enriching immune cells comprising the first and/or the second nucleic acid sequence.

Also provided is a modified immune cell produced by the method according to any one of the methods of production described above.

Further provided is a pharmaceutical composition comprising the modified immune cell according to any one of the modified immune cells described above, and a pharmaceutically acceptable carrier.

Another aspect of the present application provides a method of treating a cancer in an individual, comprising administering to the individual an effective amount of the pharmaceutical composition according to any one of the pharmaceutical compositions described above. In some embodiments, the disease is cancer. In some embodiments, the individual has a low tumor burden. In some embodiments, the method does not result in cytokine storm in the individual. In some embodiments, the individual is human.

Another aspect of the present application provides a method of reducing cytokine storm in an individual receiving treatment with an immune cell comprising an engineered receptor, comprising: (a) introducing to the immune cell a heterologous nucleic acid sequence encoding an IL-15 polypeptide comprising one or more amino acid substitutions at positions 8 and/or 62, wherein numbering of the amino acid residue positions is according to SEQ ID NO: 1, thereby providing a modified immune cell; and (b) administering to the individual an effective amount of the modified immune cell.

A further aspect of the present application provides an engineered IL-15 polypeptide comprising amino acid substitution D8E and/or T62G; wherein numbering of the amino acid residue positions is according to SEQ ID NO: 1. In some embodiments, the engineered IL-15 polypeptide comprises an amino acid sequence having at least about 90% sequence identity to an amino acid sequence selected from the group consisting of SEQ ID NOs: 5 and 7.

Another aspect of the present application provides a method of enhancing anti-tumor activity of an immune cell comprising an engineered receptor, comprising: (a) introducing to the immune cell a heterologous nucleic acid sequence encoding an IL-15 polypeptide comprising one or more amino acid substitutions at positions 3 and/or 25, wherein numbering of the amino acid residue positions is according to SEQ ID NO: 1, thereby providing a modified immune cell; and (b) administering to the individual an effective amount of the modified immune cell.

A further aspect of the present application provides an engineered IL-15 polypeptide comprising amino acid substitution V3Y and/or L25F; wherein numbering of the amino acid residue positions is according to SEQ ID NO: 1. In some embodiments, the engineered IL-15 polypeptide comprises an amino acid sequence having at least about 90% sequence identity to an amino acid sequence selected from the group consisting of SEQ ID NOs: 78 and 79.

Compositions, uses, kits and articles of manufacture comprising any one of the modified immune cells are also provided.

### BRIEF DESCRIPTION OF THE DRAWINGS

FIGS. 1A-1C show in vitro cytotoxic effects of NK cells expressing eight selected mutated constructs of secreted IL-15 armored BCMA CARs (i.e., sIL-15 m1-m8 armored BCMA CAR-NK) against BCMA-positive target cells, NCI-H929. FIG. 1A shows short-term (4 hours) in vitro cytotoxicity of sIL-15 mutant armored BCMA CAR-NK cells against target cells. FIG. 1B shows in vitro cytotoxicity (i.e., fraction of tumor cells over total cells) of sIL-15 mutant armored BCMA CAR-NK cells against target cells during eight runs of antigen stimulation. Untransduced NK cells (i.e., “UnNK”) served as controls in the experiments. During the stimulation with tumor cells, the sIL-15 mutant armored BCMA CAR-NK cells were also evaluated for expansion fold (FIG. 1C).

FIGS. 2A-2B show in vivo anti-tumor efficacy of CAR-NK cells armored with wildtype secreted IL-15 (i.e., “sIL-15 wt”) against BCMA-positive target cells, NCI-H929, in a NCG mouse model (NCI-H929-luc model) with a multiple myeloma tumor xenograft. FIGS. 2A-2B show the anti-tumor activity (FIG. 2A) and survival of mice (FIG. 2B) treated with sIL-15 wt armored NK cells, sIL-15 wt armored CD19 CAR-NK cells, and sIL-15 wt armored BCMA CAR-NK cells in mice. Untransduced NK cells combined with hIL-15 i.p. (i.e., “UnNK, i.v.+IL-15, i.p.”), a HBSS (−/−) vehicle control (i.e., “Vehicle, i.v.”), and non-tumor bearing NCG mice served as controls in the experiments.

FIGS. 3A-3C show in vivo evaluation of BCMA CAR-NK cells armored with mutated secreted IL-15 against BCMA-positive target cells, NCI-H929, in a NCG mouse model (NCI-H929-luc model) with a multiple myeloma tumor xenograft. FIG. 3A shows anti-tumor efficacy of the mutant IL-15 BCMA CAR-NK cells, and FIG. 3B shows a bioluminescence imaging (BLI) representation of the anti-tumor efficacy of the mutant IL-15 BCMA CAR-NK cells in mouse peripheral blood. FIG. 3C shows the IFN-γ secretion in mouse plasma. HBSS (−/−) vehicle control (i.e., “Vehicle”) and non-tumor bearing NCG mice served as controls in the experiments.

FIGS. 4A-4G show in vivo evaluation of BCMA CAR-NK cells armored with mutated secreted IL-15 and membrane bound wildtype IL-15 against BCMA-positive target cells, NCI-H929, in a NCG mouse model (NCI-H929-Luc model) with a high tumor burden. FIG. 4A shows anti-tumor efficacy and FIG. 4B shows BCMA CAR PK in mouse peripheral blood. FIGS. 4C-4D show the BLI and survival curve of mice treated with mutant IL-15 armored BCMA CAR-NK cells. FIGS. 4E-4G show the levels of pro-inflammatory cytokines in mice plasma, including IFN-γ (FIG. 4E), TNF-α (FIG. 4F), and GM-CSF (FIG. 4G) in mice plasma, corresponding with observations of toxicity. Untransduced NK cells combined with hIL-15 i.p. (i.e., “UnNK, i.v.+IL-15, i.p.”) served as controls in the experiments.

FIGS. 5A-5B show in vitro cytotoxic effects of NK cells expressing membrane bound mutated IL-15 (i.e., membrane bound IL-15 m6) armored BCMA CARs against BCMA-positive target cells, NCI-H929. FIG. 5A shows short-term (4 hours) in vitro cytotoxicity of sIL-15 wt armored BCMA CAR-NK cells and membrane bound IL-15 m6 armored BCMA CAR-NK cells against target cells. FIG. 5B shows long-term in vitro cytotoxicity (i.e., fraction of tumor cells over total cells) of sIL-15 wt armored BCMA CAR-NK cells and membrane bound IL-15 m6 armored BCMA CAR-NK cells against target cells during four runs of antigen stimulation. Untransduced NK cells (i.e., “UnNK”) served as controls in the experiments.

FIGS. 6A-6C show in vitro cytotoxic effects of NK cells expressing membrane bound mutated IL-15 (i.e., membrane bound IL-15 m4) armored BCMA CARs against BCMA-positive target cells, NCI-H929. FIG. 6A shows short-term (4 hours) in vitro cytotoxicity of sIL-15 wt armored BCMA CAR-NK cells and membrane bound IL-15 m4 armored BCMA CAR-NK cells against target cells. FIG. 6B shows long-term in vitro cytotoxicity (i.e., fraction of tumor cells over total cells) of sIL-15 wt armored BCMA CAR-NK cells and membrane bound IL-15 m4 armored BCMA CAR-NK cells against target cells during seven runs of antigen stimulation. Untransduced NK cells (i.e., “UnNK”) served as controls in the experiments. During the stimulation with tumor cells, the sIL-15 wt armored BCMA CAR-NK cells and membrane bound IL-15 m4 armored BCMA CAR-NK cells were also evaluated for expansion fold (FIG. 6C).

FIGS. 7A-7B show in vivo evaluation of BCMA CAR-NK cells armored with wildtype secreted IL-15 (i.e., “sIL-15 wt”) and membrane bound mutated IL-15 against BCMA-positive target cells, NCI-H929, in a NCG mouse model (NCI-H929-Luc model) with a high tumor burden. FIG. 7A shows BCMA CAR PK in mouse peripheral blood. FIG. 7B shows the survival curve of mice treated with sIL-15 wt armored BCMA CAR-NK and membrane bound mutated IL-15 (i.e., mb-4 IL-15 m6 and mb-5 IL-15 m6) armored BCMA CAR-NK cells. Untransduced NK cells combined with hIL-15 i.p. (i.e., “UnNK, i.v.+IL-15, i.p.”) served as controls in the experiments.

FIG. 8A-8B show in vitro cytotoxicity of sIL-15 m17 armored GPC3 CAR-NK cells on Huh7/Luc cells in a short-term (FIG. 8A) and long-term (FIG. 8B) cell killing assay. sIL-15 m17 armored GPC3 CAR-NK cells showed potent anti-tumor efficacy against Huh7/Luc cells in the short-term killing assay and long-term killing assay after R2 compared to sIL-15 wt armored GPC3 CAR-NK cells. “UnNK” means untransduced NK cells. R0, R2, R4 and R5 in FIG. 8B mean Rounds of target cell stimulation.

FIG. 9A-9B show in vitro cytotoxicity of sIL-15 m18 armored GPC3 CAR-NK cells on Huh7/Luc cells in a short-term (FIG. 9A) and long-term (FIG. 9B) cell killing assay. sIL-15 m18 armored GPC3 CAR-NK cells showed potent anti-tumor efficacy against Huh7/Luc cells in the short-term killing assay and long-term killing assay after R2 compared to sIL-15 wt armored GPC3 CAR-NK cells. “UnNK” means untransduced NK cells. R0, R2, R4 and R6 in FIG. 9B mean Rounds of target cell stimulation.

### DETAILED DESCRIPTION

The present application provides modified immune cells expressing a mutant IL-15 polypeptide, which have potent tumor lytic activity and improved safety profile compared to modified immune cells expressing a wildtype IL-15 polypeptide. In some embodiments, the mutant IL-15 polypeptide has reduced (i.e., weaker) binding affinity to IL-15β. In some embodiments, the IL-15 polypeptide comprises one or more amino acid substitutions at positions 8 and/or 62, wherein numbering of the amino acid residue positions is according to SEQ ID NO: 1. In some embodiments, the IL-15 polypeptide comprises one or more amino acid substitutions at positions 3 and/or 25, wherein numbering of the amino acid residue positions is according to SEQ ID NO: 1. The IL-15 polypeptide may be a membrane-bound molecule, or secreted from the modified immune cell. In some embodiments, the modified immune cells are natural killer (NK) cells and further express a chimeric antigen receptor (CAR) that specifically recognizes a target antigen of interest.

The modified immune cells described herein are based at least in part on the discovery that wildtype IL-15 armored CAR NK cells lead to excessive cytokine secretion that is toxic to the subject treated with the IL-15 armored CAR NK cells. Compared to wildtype IL-15, mutant IL-15 polypeptides having reduced binding affinity to IL15-Rβ can alleviate cytokine secretion by immune cells armored with such IL-15 polypeptides, but at the same time attenuate anti-tumor activity of the same immune cells. Modified immune cells (e.g., CAR NK cells) expressing the mutant IL-15 polypeptides described herein (e.g., D8E, T62G, V3Y and L25F mutants) retain or enhance potent anti-tumor efficacy without inducing overproduction of inflammatory cytokine(s), e.g., inducing cytokine storm, in the treated subjects.

Accordingly, one aspect of the present application provides a modified immune cell (e.g., NK cell or T cell) comprising a heterologous nucleic acid sequence encoding an IL-15 polypeptide comprising one or more amino acid substitutions at positions 8, 62, 3, and/or 25, wherein numbering of the amino acid residue positions is according to SEQ ID NO: 1. In some embodiments, the IL-15 polypeptide comprises a D8E substitution. In some embodiments, the IL-15 polypeptide comprises a T62G substitution. In some embodiments, the IL-15 polypeptide comprises a V3Y substitution. In some embodiments, the IL-15 polypeptide comprises a L25F substitution. In some embodiments, the IL-15 polypeptide is secreted by the modified immune cell. In some embodiments, the IL-15 polypeptide is bound to the cell membrane of the modified immune cell via a GPI linker. In some embodiments, the IL-15 polypeptide comprises a transmembrane domain or membrane-anchoring domain. In some embodiments, the modified immune cell further comprises an engineered receptor, such as a chimeric antigen receptor, a modified T-cell receptor, or a T-cell antigen coupler (TAC) receptor.

Also provided are compositions (such as pharmaceutical compositions), kits and articles of manufacture comprising the modified immune cells, and methods of treating a disease or condition (e.g., cancer) using the modified immune cells described herein.

### I. Definitions

As used herein, “treatment” or “treating” is an approach for obtaining beneficial or desired results including clinical results. For purposes of this disclosure, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of the disease (e.g., cancer). The methods of the present application contemplate any one or more of these aspects of treatment.

The term “prevent,” and similar words such as “prevented,” “preventing” etc., indicate an approach for preventing, inhibiting, or reducing the likelihood of the recurrence of, a disease or condition, e.g., cancer. It also refers to delaying the recurrence of a disease or condition or delaying the recurrence of the symptoms of a disease or condition. As used herein, “prevention” and similar words also includes reducing the intensity, effect, symptoms and/or burden of a disease or condition prior to recurrence of the disease or condition.

As used herein, “delaying” the development of cancer means to defer, hinder, slow, retard, stabilize, and/or postpone development of the disease. This delay can be of varying lengths of time, depending on the history of the disease and/or individual being treated. A method that “delays” development of cancer is a method that reduces probability of disease development in a given time frame and/or reduces the extent of the disease in a given time frame, when compared to not using the method. Such comparisons are typically based on clinical studies, using a statistically significant number of individuals. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to cancer progression that may be initially undetectable and includes occurrence, recurrence, and onset.

The term “effective amount” used herein refers to an amount of an agent or a combination of agents, sufficient to treat a specified disorder, condition or disease such as to ameliorate, palliate, lessen, and/or delay one or more of its symptoms. In reference to cancer, an effective amount comprises an amount sufficient to cause a tumor to shrink and/or to decrease the growth rate of the tumor (such as to suppress tumor growth) or to prevent or delay other undesired cell proliferation. In some embodiments, an effective amount is an amount sufficient to delay disease development. In some embodiments, an effective amount is an amount sufficient to prevent or delay recurrence. An effective amount can be administered in one or more administrations. The effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

As used herein, an “individual” or a “subject” refers to a mammal, including, but not limited to, human, bovine, horse, feline, canine, rodent, or primate. In some embodiments, the individual is a human.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which a heterologous nucleic acid is transferred or introduced into the host cell. A “transfected” or “transformed” or “transduced” cell is one which has been transfected, transformed or transduced with a heterologous nucleic acid. The cell includes the primary subject cell and its progeny.

“Percent (%) amino acid sequence identity” with respect to the polypeptide sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32(5):1792-1797, 2004; Edgar, R. C., BMC Bioinformatics 5(1):113, 2004).

“Chimeric antigen receptor” or “CAR” as used herein refers to genetically engineered receptors, which graft one or more antigen specificity onto cells, such as T cells. CARs are also known as “artificial T-cell receptors,” “chimeric T-cell receptors,” or “chimeric immune receptors.” In some embodiments, the CAR comprises an extracellular variable domain of an antibody specific for a tumor antigen, and an intracellular signaling domain of a T cell or other receptors, such as one or more co-stimulatory domains. “CAR-T” refers to a T cell that expresses a CAR. “CAR-NK” refers to a NK cell that expresses a CAR. As used herein, a “BCMA CAR” refers to a CAR that specifically recognizes BCMA, a “CD19 CAR” refers to a CAR that specifically recognizes CD19, and a “GPC3 CAR” refers to a CAR that specifically recognizes GPC3.

“T-cell receptor” or “TCR” as used herein refers to an endogenous or modified T-cell receptor comprising an extracellular antigen binding domain that binds to a specific antigenic peptide bound in an MHC molecule. In some embodiments, the TCR comprises a TCRα polypeptide chain and a TCR β polypeptide chain. In some embodiments, the TCR comprises a TCRγ polypeptide chain and a TCR δ polypeptide chain. In some embodiments, the TCR specifically binds a tumor antigen. “TCR-T” refers to a T cell that expresses a recombinant TCR.

“T-cell antigen coupler receptor” or “TAC receptor” as used herein refers to an engineered receptor comprising an extracellular antigen binding domain that binds to a specific antigen and a T-cell receptor (TCR) binding domain, a transmembrane domain, and an intracellular domain of a co-receptor molecule. The TAC receptor co-opts the endogenous TCR of a T cell that expressed the TAC receptor to elicit antigen-specific T-cell response against a target cell.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. The term antibody includes, but is not limited to, fragments that are capable of binding antigen, such as Fv, single-chain Fv (scFv), Fab, Fab', and (Fab')₂. The term antibody includes conventional four-chain antibodies, and single-domain antibodies, such as heavy-chain only antibodies or fragments thereof, e.g., VHH.

As use herein, the term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding.

The term “cell” includes the primary subject cell and its progeny.

It is understood that embodiments of the disclosure described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “an,” and “the” include plural referents unless the context clearly dictates otherwise.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the modified immune cells and methods of treatment described herein are specifically embraced by the present application and are disclosed herein just as if each and every combination was individually and explicitly disclosed. In addition, all subcombinations of the modified immune cells listed in the embodiments describing such variables are also specifically embraced by the present application and are disclosed herein just as if each and every such sub-combination of proteins was individually and explicitly disclosed herein.

### II. Modified Immune Cells

One aspect of the present application provides a modified immune cell comprising a heterologous nucleic acid sequence encoding an IL-15 polypeptide comprising a mutant IL-15 having one or more amino acid substitutions with respect to wildtype IL-15, wherein the IL-15 polypeptide upon expression is capable of binding to an IL-15 receptor. In some embodiments, the mutant IL-15 has reduced binding affinity to the IL-15 receptor compared to the wildtype IL-15. In some embodiments, the mutant IL-15 has reduced binding affinity, such as reduced by at least about any one of 10%, 20%, 30%, 40%, 50%, 60%, 70%, 80%, 90%, 12 fold, 14 fold, 16 fold, 20 fold, 25 fold, 30 fold, 40 fold or more, to IL-15Rβ compared to a wildtype IL-15. In some embodiments, the mutant IL-15 has reduced binding affinity, such as reduced by about 10 fold to about 20 fold, to IL-15Rβ compared to a wildtype IL-15. In some embodiments, the mutant IL-15 induces a reduced level of inflammatory cytokine (e.g., IFN-γ, TNF-α, and/or GM-CSF) secretion by the modified immune cell, e.g., such as reduced by at least about any one of 10%, 20%, 30%, 40%, 50%, 60%, 70%, 80%, 90%, 2 fold, 5 fold, 10 fold, 20 fold, 50 fold, 100 fold, 200 fold, 500 fold, 1000 fold or more, compared to a wildtype IL-15. In some embodiments, the modified immune cell has reduced toxicity in vivo when administered to an individual compared to a modified immune cell that comprises a heterologous nucleic acid encoding a wildtype IL-15 polypeptide. In some embodiments, the modified immune cell has improved safety in vivo when administered to an individual compared to a modified immune cell that comprises a heterologous nucleic acid encoding a wildtype IL-15 polypeptide. In some embodiments, the individual receiving the modified immune cell does not suffer from cytokine storm. In some embodiments, the modified immune cell comprising a heterologous nucleic acid sequence encoding an IL-15 polypeptide comprising a mutant IL-15 having one or more amino acid substitutions with respect to wildtype IL-15 has enhanced anti-tumor activity, compared to a modified immune cell that comprises a heterologous nucleic acid encoding a wildtype IL-15 polypeptide. In some embodiments, the IL-15 polypeptide is secreted. In some embodiments, the IL-15 polypeptide is membrane bound. In some embodiments, the modified immune cell further comprises an engineered receptor, such as a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a dendritic cell (DC)—activated T cell.

In some embodiments, there is provided a modified immune cell comprising a heterologous nucleic acid sequence encoding an IL-15 polypeptide comprising an amino acid substitution at position 62, wherein numbering of the amino acid residue positions is according to SEQ ID NO: 1. In some embodiments, the IL-15 polypeptide comprises an amino acid residue selected from the group consisting of Glycine (G), Isoleucine (I), Glutamine (Q), Valine (V), Proline (P), Leucine (L), Alanine (A), Serine (S) and Tyrosine (Y) at position 62. In some embodiments, the amino acid substitution at position 62 is selected from the group consisting of T62G, T62I, T62Q, T62V, T62P, T62L, T62A, T62S and T62Y. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to an amino acid sequence selected from the group consisting of SEQ ID NOs: 7-8 and 11-17. In some embodiments, the IL-15 polypeptide comprises an amino acid sequence selected from the group consisting of SEQ ID NOs: 7-8 and 11-17. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a modified immune cell comprising a heterologous nucleic acid sequence encoding an IL-15 polypeptide comprising a G at position 62, wherein numbering of the amino acid residue positions is according to SEQ ID NO: 1. In some embodiments, the IL-15 polypeptide comprises a T62G substitution. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 7. In some embodiments, the IL-15 polypeptide comprises SEQ ID NO: 7. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a modified immune cell comprising a heterologous nucleic acid sequence encoding an IL-15 polypeptide comprising an amino acid substitution at position 8, wherein numbering of the amino acid residue positions is according to SEQ ID NO: 1. In some embodiments, the IL-15 polypeptide comprises Glutamic acid (E) at position 8. In some embodiments, the amino acid substitution at position 8 is D8E. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 5. In some embodiments, the IL-15 polypeptide comprises SEQ ID NO: 5. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a modified immune cell comprising a heterologous nucleic acid sequence encoding an IL-15 polypeptide comprising an amino acid substitution at position 3, wherein numbering of the amino acid residue positions is according to SEQ ID NO: 1. In some embodiments, the IL-15 polypeptide comprises Tyrosine (Y) at position 3. In some embodiments, the amino acid substitution at position 3 is V3Y. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 78. In some embodiments, the IL-15 polypeptide comprises SEQ ID NO: 78. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a modified immune cell comprising a heterologous nucleic acid sequence encoding an IL-15 polypeptide comprising an amino acid substitution at position 25, wherein numbering of the amino acid residue positions is according to SEQ ID NO: 1. In some embodiments, the IL-15 polypeptide comprises Glutamic acid (E) or Phenylalanine (F) at position 25. In some embodiments, the amino acid substitution at position 25 is L25E. In some embodiments, the amino acid substitution at position 25 is L25F. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 79. In some embodiments, the IL-15 polypeptide comprises SEQ ID NO: 79. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a modified immune cell comprising a heterologous nucleic acid sequence encoding a secreted IL-15 polypeptide comprising one or more amino acid substitutions at positions 8, 62, 3 and/or 25, wherein numbering of the amino acid residue positions is according to SEQ ID NO: 1. In some embodiments, the IL-15 polypeptide has reduced binding affinity an IL-15 receptor (e.g., IL-15Rα and/or IL-15Rβ/γc) compared to a wildtype IL-15 polypeptide. In some embodiments, the IL-15 polypeptide comprises an amino acid substitution at position 62. In some embodiments, the amino acid substitution at position 62 is selected from the group consisting of T62G, T62I, T62Q, T62V, T62P, T62L, T62A, T62S and T62Y. In some embodiments, the amino acid substitution at position 62 is T62G. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 7. In some embodiments, the IL-15 polypeptide comprises SEQ ID NO: 7. In some embodiments, the IL-15 polypeptide comprises an amino acid substitution at position 8. In some embodiments, the amino acid substitution at position 8 is D8E. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 5. In some embodiments, the IL-15 polypeptide comprises SEQ ID NO: 5. In some embodiments, the IL-15 polypeptide comprises an amino acid substitution at position 3. In some embodiments, the amino acid substitution at position 3 is V3Y. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 78. In some embodiments, the IL-15 polypeptide comprises SEQ ID NO: 78. In some embodiments, the IL-15 polypeptide comprises an amino acid substitution at position 25. In some embodiments, the amino acid substitution at position 25 is selected from the group consisting of L25E and L25F. In some embodiments, the amino acid substitution at position 25 is L25F. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 79. In some embodiments, the IL-15 polypeptide comprises SEQ ID NO: 79. In some embodiments, the IL-15 polypeptide comprises amino acid substitutions at both position 8 and position 62. In some embodiments, the IL-15 polypeptide is a fusion protein comprising an IL-15 fragment fused to an extracellular domain of IL-15Rα or a Sushi domain of IL-15 Ra. In some embodiments, the IL-15 polypeptide is a fusion protein comprising an amino acid sequence of SEQ ID NOs: 57 or 58. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a modified immune cell comprising a heterologous nucleic acid sequence encoding a membrane bound IL-15 polypeptide comprising one or more amino acid substitutions at positions 8, 62, 3 and/or 25, wherein numbering of the amino acid residue positions is according to SEQ ID NO: 1, wherein the IL-15 polypeptide comprises a glycosylphosphatidylinositol (GPI)—anchoring peptide sequence. In some embodiments, the TL-15 polypeptide comprises an amino acid substitution at position 62. In some embodiments, the amino acid substitution at position 62 is selected from the group consisting of T62G, T62I, T62Q, T62V, T62P, T62L, T62A, T62S and T62Y. In some embodiments, the amino acid substitution at position 62 is T62G. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 7. In some embodiments, the IL-15 polypeptide comprises SEQ ID NO: 7. In some embodiments, the IL-15 polypeptide comprises an amino acid substitution at position 8. In some embodiments, the amino acid substitution at position 8 is D8E. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 5. In some embodiments, the IL-15 polypeptide comprises SEQ ID NO: 5. In some embodiments, the IL-15 polypeptide comprises an amino acid substitution at position 3. In some embodiments, the amino acid substitution at position 3 is V3Y. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 78. In some embodiments, the IL-15 polypeptide comprises SEQ ID NO: 78. In some embodiments, the IL-15 polypeptide comprises an amino acid substitution at position 25. In some embodiments, the amino acid substitution at position 25 is selected from the group consisting of L25E and L25F. In some embodiments, the amino acid substitution at position 25 is L25F. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 79. In some embodiments, the IL-15 polypeptide comprises SEQ ID NO: 79. In some embodiments, the IL-15 polypeptide comprises amino acid substitutions at both position 8 and position 62. In some embodiments, the GPI-anchoring peptide sequence is attached to a GPI linker. In some embodiments, the GPI-anchoring peptide sequence is located at the C-terminus of the IL-15 polypeptide. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a modified immune cell comprising a heterologous nucleic acid sequence encoding an IL-15 polypeptide, which is a fusion protein comprising an IL-15 fragment fused to a second polypeptide fragment, wherein the IL-15 fragment comprises one or more amino acid substitutions at positions 8 62, 3 and/or 25, wherein numbering of the amino acid residue positions is according to SEQ ID NO: 1. In some embodiments, the IL-15 fragment comprises an amino acid substitution at position 62. In some embodiments, the amino acid substitution at position 62 is selected from the group consisting of T62G, T62I, T62Q, T62V, T62P, T62L, T62A, T62S and T62Y. In some embodiments, the amino acid substitution at position 62 is T62G. In some embodiments, the IL-15 fragment comprises an amino acid sequence having at least about 90% (e.g., at least about any one of 91%, 92%, 93%, 94%, 95%, 96%, 97%, 98%, 99%, or higher) sequence identity to SEQ ID NO: 7. In some embodiments, the IL-15 fragment comprises SEQ ID NO: 7. In some embodiments, the IL-15 fragment comprises an amino acid substitution at position 8. In some embodiments, the amino acid substitution at position 8 is D8E. In some embodiments, the IL-15 fragment comprises an amino acid sequence having at least about 90% (e.g., at least about any one of 91%, 92%, 93%, 94%, 95%, 96%, 97%, 98%, 99%, or higher) sequence identity to SEQ ID NO: 5. In some embodiments, the IL-15 fragment comprises SEQ ID NO: 5. In some embodiments, the IL-15 polypeptide comprises an amino acid substitution at position 3. In some embodiments, the amino acid substitution at position 3 is V3Y. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 78. In some embodiments, the IL-15 polypeptide comprises SEQ ID NO: 78. In some embodiments, the IL-15 polypeptide comprises an amino acid substitution at position 25. In some embodiments, the amino acid substitution at position 25 is selected from the group consisting of L25E and L25F. In some embodiments, the amino acid substitution at position 25 is L25F. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 79. In some embodiments, the IL-15 polypeptide comprises SEQ ID NO: 79. In some embodiments, the IL-15 fragment comprises amino acid substitutions at both position 8 and position 62. In some embodiments, the second polypeptide fragment is selected from the group consisting of IL-15Rα, an extracellular domain of IL-15Rα, a Sushi domain of IL-15Rα, a transmembrane domain of IL-15Rα, IL-15Rβ, common gamma chain (γc), an engineered receptor (e.g., CAR, TCR or TAC) and combinations thereof. In some embodiments, the second polypeptide fragment comprises an amino acid sequence selected from the group consisting of SEQ ID NOs: 50-55. In some embodiments, the IL-15 fragment is fused to the second polypeptide fragment via a peptide linker. In some embodiments, the IL-15 polypeptide described herein above comprises an amino acid sequence selected from the group consisting of SEQ ID NOs: 57-64, 76 and 77. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a modified immune cell comprising a heterologous nucleic acid sequence encoding a membrane bound IL-15 polypeptide comprising one or more amino acid substitutions at positions 8, 62, 3 and/or 25, wherein numbering of the amino acid residue positions is according to SEQ ID NO: 1, and wherein the IL-15 polypeptide comprises a transmembrane domain. In some embodiments, the IL-15 polypeptide comprises an amino acid substitution at position 62. In some embodiments, the amino acid substitution at position 62 is selected from the group consisting of T62G, T62I, T62Q, T62V, T62P, T62L, T62A, T62S and T62Y. In some embodiments, the amino acid substitution at position 62 is T62G. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 7. In some embodiments, the IL-15 polypeptide comprises SEQ ID NO: 7. In some embodiments, the IL-15 polypeptide comprises an amino acid substitution at position 8. In some embodiments, the amino acid substitution at position 8 is D8E. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 5. In some embodiments, the IL-15 polypeptide comprises SEQ ID NO: 5. In some embodiments, the IL-15 polypeptide comprises an amino acid substitution at position 3. In some embodiments, the amino acid substitution at position 3 is V3Y. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 78. In some embodiments, the IL-15 polypeptide comprises SEQ ID NO: 78. In some embodiments, the IL-15 polypeptide comprises an amino acid substitution at position 25. In some embodiments, the amino acid substitution at position 25 is selected from the group consisting of L25E and L25F. In some embodiments, the amino acid substitution at position 25 is L25F. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 79. In some embodiments, the IL-15 polypeptide comprises SEQ ID NO: 79. In some embodiments, the IL-15 polypeptide comprises amino acid substitutions at both position 8 and position 62. In some embodiments, the transmembrane domain is a transmembrane domain of IL-15Rα. In some embodiments, the IL-15 polypeptide further comprises an intracellular domain. In some embodiments, the IL-15 polypeptide comprises: (a) an antigen-binding domain; (b) an IL-15 fragment; (c) a transmembrane domain; and (d) an intracellular domain. In some embodiments, the antigen-binding domain is at the N-terminus of the IL-15 fragment. In some embodiments, the antigen-binding domain is at the C-terminus of the IL-15 fragment. In some embodiments, the transmembrane domain is a CD4, CD3, CD8a, or CD28 transmembrane domain. In some embodiments, the IL-15 polypeptide further comprises a hinge domain, such as a hinge domain derived from CD8. In some embodiments, the intracellular domain comprises a primary intracellular signaling domain, such as an intracellular signaling domain of CD3ζ. In some embodiments, the intracellular domain comprises a co-stimulatory signaling domain. In some embodiments, the co-stimulatory signaling domain is derived from a co-stimulatory molecule selected from the group consisting of CD27, CD28, 4-1BB, OX40, DAP10, CD30, CD40, CD3, LFA-1, CD2, CD7, LIGHT, NKG2C, B7-H3, ligands of CD83 and combinations thereof. In some embodiments, the IL-15 polypeptide comprises SEQ ID NO: 65, 66 or 75. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a modified immune cell comprising a heterologous nucleic acid sequence encoding a membrane bound IL-15 polypeptide comprising one or more amino acid substitutions at positions 8, 62, 3 and/or 25, wherein numbering of the amino acid residue positions is according to SEQ ID NO: 1, and wherein the IL-15 polypeptide comprises a membrane anchoring domain. In some embodiments, the IL-15 polypeptide comprises an amino acid substitution at position 62. In some embodiments, the amino acid substitution at position 62 is selected from the group consisting of T62G, T62I, T62Q, T62V, T62P, T62L, T62A, T62S and T62Y. In some embodiments, the amino acid substitution at position 62 is T62G. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 7. In some embodiments, the IL-15 polypeptide comprises SEQ ID NO: 7. In some embodiments, the IL-15 polypeptide comprises an amino acid substitution at position 8. In some embodiments, the amino acid substitution at position 8 is D8E. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 5. In some embodiments, the IL-15 polypeptide comprises SEQ ID NO: 5. In some embodiments, the IL-15 polypeptide comprises an amino acid substitution at position 3. In some embodiments, the amino acid substitution at position 3 is V3Y. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 78. In some embodiments, the IL-15 polypeptide comprises SEQ ID NO: 78. In some embodiments, the IL-15 polypeptide comprises an amino acid substitution at position 25. In some embodiments, the amino acid substitution at position 25 is selected from the group consisting of L25E and L25F. In some embodiments, the amino acid substitution at position 25 is L25F. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 79. In some embodiments, the IL-15 polypeptide comprises SEQ ID NO: 79. In some embodiments, the IL-15 polypeptide comprises amino acid substitutions at both position 8 and position 62. In some embodiments, the membrane anchoring domain is derived from a molecule selected from the group consisting of an IL-15Rα, a transmembrane domain of IL-15Rα, an IL-15Rβ, an extracellular domain of IL-15Rα, a Sushi domain of IL-15Rα, an extracellular domain of IL-15Rβ, a common gamma chain (γc) and combinations thereof. In some embodiments, the IL-15 polypeptide comprises an amino acid sequence selected from the group consisting of SEQ ID NOs: 59-64, 76 and 77. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a modified immune cell (e.g., NK cell or T cell) comprising a first heterologous nucleic acid sequence encoding an IL-15 polypeptide comprising one or more amino acid substitutions at positions 8, 62, 3 and/or 25, wherein numbering of the amino acid residue positions is according to SEQ ID NO: 1; and a second heterologous nucleic acid sequence encoding an engineered receptor. In some embodiments, the IL-15 polypeptide comprises an amino acid substitution at position 62. In some embodiments, the amino acid substitution at position 62 is selected from the group consisting of T62G, T62I, T62Q, T62V, T62P, T62L, T62A, T62S and T62Y. In some embodiments, the amino acid substitution at position 62 is T62G. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 7. In some embodiments, the IL-15 polypeptide comprises SEQ ID NO: 7. In some embodiments, the IL-15 polypeptide comprises an amino acid substitution at position 8. In some embodiments, the amino acid substitution at position 8 is D8E. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 5. In some embodiments, the IL-15 polypeptide comprises SEQ ID NO: 5. In some embodiments, the IL-15 polypeptide comprises an amino acid substitution at position 3. In some embodiments, the amino acid substitution at position 3 is V3Y. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 78. In some embodiments, the IL-15 polypeptide comprises SEQ ID NO: 78. In some embodiments, the IL-15 polypeptide comprises an amino acid substitution at position 25. In some embodiments, the amino acid substitution at position 25 is selected from the group consisting of L25E and L25F. In some embodiments, the amino acid substitution at position 25 is L25F. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 79. In some embodiments, the IL-15 polypeptide comprises SEQ ID NO: 79. In some embodiments, the IL-15 polypeptide comprises amino acid substitutions at both position 8 and position 62. In some embodiments, the engineered receptor is a CAR, such as a BCMA CAR, a CD19 CAR, or a GPC3 CAR. In some embodiments, the engineered receptor is an engineered TCR. In some embodiments, the engineered receptor is a TAC receptor. In some embodiments, the first nucleic acid sequence and the second nucleic acid sequence are on the same vector or separate vectors. In some embodiments, the first nucleic acid sequence and the second nucleic acid sequence are operably linked to the same promoter or separate promoters. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a modified immune cell (e.g., NK cell or T cell) comprising a first heterologous nucleic acid sequence encoding a secreted IL-15 polypeptide comprising one or more amino acid substitutions at positions 8, 62, 3 and/or 25, wherein numbering of the amino acid residue positions is according to SEQ ID NO: 1; and a second heterologous nucleic acid sequence encoding an engineered receptor. In some embodiments, the TL-15 polypeptide comprises an amino acid substitution at position 62. In some embodiments, the amino acid substitution at position 62 is selected from the group consisting of T62G, T62I, T62Q, T62V, T62P, T62L, T62A, T62S and T62Y. In some embodiments, the amino acid substitution at position 62 is T62G. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 7. In some embodiments, the IL-15 polypeptide comprises SEQ ID NO: 7. In some embodiments, the IL-15 polypeptide comprises an amino acid substitution at position 8. In some embodiments, the amino acid substitution at position 8 is D8E. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 5. In some embodiments, the IL-15 polypeptide comprises SEQ ID NO: 5. In some embodiments, the IL-15 polypeptide comprises an amino acid substitution at position 3. In some embodiments, the amino acid substitution at position 3 is V3Y. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 78. In some embodiments, the IL-15 polypeptide comprises SEQ ID NO: 78. In some embodiments, the IL-15 polypeptide comprises an amino acid substitution at position 25. In some embodiments, the amino acid substitution at position 25 is selected from the group consisting of L25E and L25F. In some embodiments, the amino acid substitution at position 25 is L25F. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 79. In some embodiments, the IL-15 polypeptide comprises SEQ ID NO: 79. In some embodiments, the IL-15 polypeptide comprises amino acid substitutions at both position 8 and position 62. In some embodiments, the engineered receptor is a CAR, such as a BCMA CAR, a CD19 CAR, or a GPC3 CAR. In some embodiments, the engineered receptor is an engineered TCR. In some embodiments, the engineered receptor is a TAC receptor. In some embodiments, the first nucleic acid sequence and the second nucleic acid sequence are on the same vector or separate vectors. In some embodiments, the first nucleic acid sequence and the second nucleic acid sequence are operably linked to the same promoter or separate promoters. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell. In some embodiments, the modified immune cell comprising a nucleic acid sequence encoding the amino acid sequence selected from the group consisting of SEQ ID NOs: 31-38, 42-49, 83 and 84.

In some embodiments, there is provided a modified immune cell (e.g., NK cell or T cell) comprising a first heterologous nucleic acid sequence encoding a membrane bound IL-15 polypeptide comprising a GPI-anchoring peptide sequence, wherein the IL-15 polypeptide comprises one or more amino acid substitutions at positions 8, 62, 3 and/or 25, wherein numbering of the amino acid residue positions is according to SEQ ID NO: 1; and a second heterologous nucleic acid sequence encoding an engineered receptor. In some embodiments, the IL-15 polypeptide comprises an amino acid substitution at position 62. In some embodiments, the amino acid substitution at position 62 is selected from the group consisting of T62G, T62I, T62Q, T62V, T62P, T62L, T62A, T62S and T62Y. In some embodiments, the amino acid substitution at position 62 is T62G. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 7. In some embodiments, the IL-15 polypeptide comprises SEQ ID NO: 7. In some embodiments, the IL-15 polypeptide comprises an amino acid substitution at position 8. In some embodiments, the amino acid substitution at position 8 is D8E. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 5. In some embodiments, the IL-15 polypeptide comprises SEQ ID NO: 5. In some embodiments, the IL-15 polypeptide comprises an amino acid substitution at position 3. In some embodiments, the amino acid substitution at position 3 is V3Y. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 78. In some embodiments, the IL-15 polypeptide comprises SEQ ID NO: 78. In some embodiments, the IL-15 polypeptide comprises an amino acid substitution at position 25. In some embodiments, the amino acid substitution at position 25 is selected from the group consisting of L25E and L25F. In some embodiments, the amino acid substitution at position 25 is L25F. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 79. In some embodiments, the IL-15 polypeptide comprises SEQ ID NO: 79. In some embodiments, the IL-15 polypeptide comprises amino acid substitutions at both position 8 and position 62. In some embodiments, the GPI-anchoring peptide sequence is attached to a GPI linker. In some embodiments, the GPI-anchoring peptide sequence is located at the C-terminus of the IL-15 polypeptide. In some embodiments, the engineered receptor is a CAR, such as a BCMA CAR, a CD19 CAR, or a GPC3 CAR. In some embodiments, the engineered receptor is an engineered TCR. In some embodiments, the engineered receptor is a TAC receptor. In some embodiments, the first nucleic acid sequence and the second nucleic acid sequence are on the same vector or separate vectors. In some embodiments, the first nucleic acid sequence and the second nucleic acid sequence are operably linked to the same promoter or separate promoters. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a modified immune cell (e.g., NK cell or T cell) comprising a first heterologous nucleic acid sequence encoding a membrane bound IL-15 polypeptide comprising a transmembrane domain, wherein the IL-15 polypeptide comprises one or more amino acid substitutions at positions 8, 62, 3 and/or 25, wherein numbering of the amino acid residue positions is according to SEQ ID NO: 1; and a second heterologous nucleic acid sequence encoding an engineered receptor. In some embodiments, the IL-15 polypeptide comprises an amino acid substitution at position 62. In some embodiments, the amino acid substitution at position 62 is selected from the group consisting of T62G, T62I, T62Q, T62V, T62P, T62L, T62A, T62S and T62Y. In some embodiments, the amino acid substitution at position 62 is T62G. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 7. In some embodiments, the IL-15 polypeptide comprises SEQ ID NO: 7. In some embodiments, the IL-15 polypeptide comprises an amino acid substitution at position 8. In some embodiments, the amino acid substitution at position 8 is D8E. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 5. In some embodiments, the IL-15 polypeptide comprises SEQ ID NO: 5. In some embodiments, the IL-15 polypeptide comprises an amino acid substitution at position 3. In some embodiments, the amino acid substitution at position 3 is V3Y. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 78. In some embodiments, the IL-15 polypeptide comprises SEQ ID NO: 78. In some embodiments, the IL-15 polypeptide comprises an amino acid substitution at position 25. In some embodiments, the amino acid substitution at position 25 is selected from the group consisting of L25E and L25F. In some embodiments, the amino acid substitution at position 25 is L25F. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 79. In some embodiments, the IL-15 polypeptide comprises SEQ ID NO: 79. In some embodiments, the IL-15 polypeptide comprises amino acid substitutions at both position 8 and position 62. In some embodiments, the transmembrane domain is a transmembrane domain of IL-15Rα. In some embodiments, the IL-15 polypeptide further comprises an intracellular domain. In some embodiments, the engineered receptor is a CAR, such as a BCMA CAR, a CD19 CAR, or a GPC3 CAR. In some embodiments, the engineered receptor is an engineered TCR. In some embodiments, the engineered receptor is a TAC receptor. In some embodiments, the first nucleic acid sequence and the second nucleic acid sequence are on the same vector or separate vectors. In some embodiments, the first nucleic acid sequence and the second nucleic acid sequence are operably linked to the same promoter or separate promoters. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a modified immune cell (e.g., NK cell or T cell) comprising a heterologous nucleic acid sequence encoding an engineered receptor comprising: (a) an antigen-binding domain; (b) an TL-15 fragment; (c) a transmembrane domain; and (d) an intracellular domain; wherein the IL-15 fragment comprises one or more amino acid substitutions at positions 8, 62, 3 and/or 25, wherein numbering of the amino acid residue positions is according to SEQ ID NO: 1. In some embodiments, the IL-15 polypeptide comprises an amino acid substitution at position 62. In some embodiments, the amino acid substitution at position 62 is selected from the group consisting of T62G, T62I, T62Q, T62V, T62P, T62L, T62A, T62S and T62Y. In some embodiments, the amino acid substitution at position 62 is T62G. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 7. In some embodiments, the IL-15 polypeptide comprises SEQ ID NO: 7. In some embodiments, the IL-15 polypeptide comprises an amino acid substitution at position 8. In some embodiments, the amino acid substitution at position 8 is D8E. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 5. In some embodiments, the IL-15 polypeptide comprises SEQ ID NO: 5. In some embodiments, the IL-15 polypeptide comprises an amino acid substitution at position 3. In some embodiments, the amino acid substitution at position 3 is V3Y. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 78. In some embodiments, the IL-15 polypeptide comprises SEQ ID NO: 78. In some embodiments, the IL-15 polypeptide comprises an amino acid substitution at position 25. In some embodiments, the amino acid substitution at position 25 is selected from the group consisting of L25E and L25F. In some embodiments, the amino acid substitution at position 25 is L25F. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 79. In some embodiments, the IL-15 polypeptide comprises SEQ ID NO: 79. In some embodiments, the IL-15 polypeptide comprises amino acid substitutions at both position 8 and position 62. In some embodiments, the antigen-binding domain is at the N-terminus of the IL-15 fragment. In some embodiments, the antigen-binding domain is at the C-terminus of the IL-15 fragment. In some embodiments, the transmembrane domain is a CD4, CD3, CD8a, or CD28 transmembrane domain. In some embodiments, the IL-15 polypeptide further comprises a hinge domain, such as a hinge domain derived from CD8a. In some embodiments, the intracellular domain comprises a primary intracellular signaling domain, such as an intracellular signaling domain of CD3ζ. In some embodiments, the intracellular domain comprises a co-stimulatory signaling domain. In some embodiments, the co-stimulatory signaling domain is derived from a co-stimulatory molecule selected from the group consisting of CD27, CD28, 4-1BB, OX40, DAP10, CD30, CD40, CD3, LFA-1, CD2, CD7, LIGHT, NKG2C, B7-H3, ligands of CD83 and combinations thereof. In some embodiments, the intracellular domain comprises a co-stimulatory signaling domain of 4-1BB and a primary intracellular signaling domain of CD3ζ. In some embodiments, the antigen-binding domain specifically binds BCMA, CD19, or GPC3. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell. In some embodiments, the modified immune cell comprising a heterologous nucleic acid sequence encoding an engineered receptor comprising SEQ ID NO: 65, 66 or 75.

In some embodiments, there is provided a modified immune cell (e.g., NK cell or T cell) comprising a first heterologous nucleic acid sequence encoding a membrane bound IL-15 polypeptide comprising a membrane anchoring domain, wherein the IL-15 polypeptide comprises one or more amino acid substitutions at positions 8, 62, 3 and/or 25, wherein numbering of the amino acid residue positions is according to SEQ ID NO: 1; and a second heterologous nucleic acid sequence encoding an engineered receptor. In some embodiments, the IL-15 polypeptide comprises an amino acid substitution at position 62. In some embodiments, the amino acid substitution at position 62 is selected from the group consisting of T62G, T62I, T62Q, T62V, T62P, T62L, T62A, T62S and T62Y. In some embodiments, the amino acid substitution at position 62 is T62G. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 7. In some embodiments, the IL-15 polypeptide comprises SEQ ID NO: 7. In some embodiments, the IL-15 polypeptide comprises an amino acid substitution at position 8. In some embodiments, the amino acid substitution at position 8 is D8E. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 5. In some embodiments, the IL-15 polypeptide comprises SEQ ID NO: 5. In some embodiments, the IL-15 polypeptide comprises an amino acid substitution at position 3. In some embodiments, the amino acid substitution at position 3 is V3Y. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 78. In some embodiments, the IL-15 polypeptide comprises SEQ ID NO: 78. In some embodiments, the IL-15 polypeptide comprises an amino acid substitution at position 25. In some embodiments, the amino acid substitution at position 25 is selected from the group consisting of L25E and L25F. In some embodiments, the amino acid substitution at position 25 is L25F. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 79. In some embodiments, the IL-15 polypeptide comprises SEQ ID NO: 79. In some embodiments, the IL-15 polypeptide comprises amino acid substitutions at both position 8 and position 62. In some embodiments, the membrane anchoring domain is derived from a molecule selected from the group consisting of an IL-15Rα, a transmembrane domain of IL-15Rα, an IL-15Rβ, an extracellular domain of IL-15Rα, a Sushi domain of IL-15Rα, an extracellular domain of IL-15Rβ, a common gamma chain (γc) and combinations thereof. In some embodiments, the IL-15 polypeptide comprises an amino acid sequence selected from the group consisting of SEQ ID NOs: 59-64, 76 and 77. In some embodiments, the engineered receptor is a CAR, such as a BCMA CAR, a CD19 CAR, or a GPC3 CAR. In some embodiments, the engineered receptor is an engineered TCR. In some embodiments, the engineered receptor is a TAC receptor. In some embodiments, the first nucleic acid sequence and the second nucleic acid sequence are on the same vector or separate vectors. In some embodiments, the first nucleic acid sequence and the second nucleic acid sequence are operably linked to the same promoter or separate promoters. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a modified immune cell (e.g., NK cell or T cell) comprising a first heterologous nucleic acid sequence encoding an IL-15 polypeptide, which is a fusion protein comprising an IL-15 fragment fused to a second polypeptide fragment, wherein the IL-15 polypeptide comprises one or more amino acid substitutions at positions 8, 62, 3 and/or 25, wherein numbering of the amino acid residue positions is according to SEQ ID NO: 1; and a second heterologous nucleic acid sequence encoding an engineered receptor. In some embodiments, the IL-15 polypeptide comprises an amino acid substitution at position 62. In some embodiments, the amino acid substitution at position 62 is selected from the group consisting of T62G, T62I, T62Q, T62V, T62P, T62L, T62A, T62S and T62Y. In some embodiments, the amino acid substitution at position 62 is T62G. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 7. In some embodiments, the IL-15 polypeptide comprises SEQ ID NO: 7. In some embodiments, the IL-15 polypeptide comprises an amino acid substitution at position 8. In some embodiments, the amino acid substitution at position 8 is D8E. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 5. In some embodiments, the IL-15 polypeptide comprises SEQ ID NO: 5. In some embodiments, the IL-15 polypeptide comprises an amino acid substitution at position 3. In some embodiments, the amino acid substitution at position 3 is V3Y. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 78. In some embodiments, the IL-15 polypeptide comprises SEQ ID NO: 78. In some embodiments, the IL-15 polypeptide comprises an amino acid substitution at position 25. In some embodiments, the amino acid substitution at position 25 is selected from the group consisting of L25E and L25F. In some embodiments, the amino acid substitution at position 25 is L25F. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 79. In some embodiments, the IL-15 polypeptide comprises SEQ ID NO: 79. In some embodiments, the IL-15 polypeptide comprises amino acid substitutions at both position 8 and position 62. In some embodiments, the second polypeptide fragment is selected from the group consisting of IL-15Rα, an extracellular domain of IL-15Rα, a Sushi domain of IL-15Rα, a transmembrane domain of IL-15Rα, IL-15Rβ, common gamma chain (γc), an engineered receptor (e.g., CAR, TCR or TAC) and combinations thereof. In some embodiments, the second polypeptide fragment comprises an amino acid sequence selected from the group consisting of SEQ ID NOs: 50-55. In some embodiments, the IL-15 fragment is fused to the second polypeptide fragment via a peptide linker. In some embodiments, the IL-15 polypeptide described herein above comprises an amino acid sequence selected from the group consisting of SEQ ID NOs: 57-64, 76 and 77. In some embodiments, the engineered receptor is a CAR, such as a BCMA CAR, a CD19 CAR, or a GPC3 CAR. In some embodiments, the engineered receptor is an engineered TCR. In some embodiments, the engineered receptor is a TAC receptor. In some embodiments, the first nucleic acid sequence and the second nucleic acid sequence are on the same vector or separate vectors. In some embodiments, the first nucleic acid sequence and the second nucleic acid sequence are operably linked to the same promoter or separate promoters. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a CAR-expressing immune cell (e.g., CAR-NK cell or CAR-T cell) comprising a heterologous nucleic acid sequence encoding an IL-15 polypeptide comprising one or more amino acid substitutions at positions 8, 62, 3 and/or 25, wherein numbering of the amino acid residue positions is according to SEQ ID NO: 1. In some embodiments, the IL-15 polypeptide comprises an amino acid substitution at position 62. In some embodiments, the amino acid substitution at position 62 is selected from the group consisting of T62G, T62I, T62Q, T62V, T62P, T62L, T62A, T62S and T62Y. In some embodiments, the amino acid substitution at position 62 is T62G. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 7. In some embodiments, the IL-15 polypeptide comprises SEQ ID NO: 7. In some embodiments, the IL-15 polypeptide comprises an amino acid substitution at position 8. In some embodiments, the amino acid substitution at position 8 is D8E. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 5. In some embodiments, the IL-15 polypeptide comprises SEQ ID NO: 5. In some embodiments, the IL-15 polypeptide comprises an amino acid substitution at position 3. In some embodiments, the amino acid substitution at position 3 is V3Y. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 78. In some embodiments, the IL-15 polypeptide comprises SEQ ID NO: 78. In some embodiments, the IL-15 polypeptide comprises an amino acid substitution at position 25. In some embodiments, the amino acid substitution at position 25 is selected from the group consisting of L25E and L25F. In some embodiments, the amino acid substitution at position 25 is L25F. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 79. In some embodiments, the IL-15 polypeptide comprises SEQ ID NO: 79. In some embodiments, the IL-15 polypeptide comprises amino acid substitutions at both position 8 and position 62. In some embodiments, the IL-15 polypeptide is secreted. In some embodiments, the IL-15 polypeptide is membrane bound. In some embodiments, the IL-15 polypeptide comprises a GPI-anchoring peptide sequence. In some embodiments, the IL-15 polypeptide comprises a transmembrane domain. In some embodiments, the IL-15 polypeptide comprises a membrane anchoring domain. In some embodiments, the IL-15 polypeptide is a fusion protein comprising an IL-15 fragment fused to a second polypeptide fragment. In some embodiments, the second polypeptide fragment is selected from the group consisting of IL-15Rα, an extracellular domain of IL-15Rα, a Sushi domain of IL-15Rα, a transmembrane domain of IL-15Rα, IL-15Rβ, common gamma chain (γc), an engineered receptor (e.g., CAR, TCR or TAC) and combinations thereof. In some embodiments, the immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a TCR-expressing immune cell (e.g., TCR-T cell or TCR-NK cell) comprising a heterologous nucleic acid sequence encoding an IL-15 polypeptide comprising one or more amino acid substitutions at positions 8, 62, 3 and/or 25, wherein numbering of the amino acid residue positions is according to SEQ ID NO: 1. In some embodiments, the IL-15 polypeptide comprises an amino acid substitution at position 62. In some embodiments, the amino acid substitution at position 62 is selected from the group consisting of T62G, T62I, T62Q, T62V, T62P, T62L, T62A, T62S and T62Y. In some embodiments, the amino acid substitution at position 62 is T62G. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 7. In some embodiments, the IL-15 polypeptide comprises SEQ ID NO: 7. In some embodiments, the IL-15 polypeptide comprises an amino acid substitution at position 8. In some embodiments, the amino acid substitution at position 8 is D8E. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 5. In some embodiments, the IL-15 polypeptide comprises SEQ ID NO: 5. In some embodiments, the IL-15 polypeptide comprises an amino acid substitution at position 3. In some embodiments, the amino acid substitution at position 3 is V3Y. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 78. In some embodiments, the IL-15 polypeptide comprises SEQ ID NO: 78. In some embodiments, the IL-15 polypeptide comprises an amino acid substitution at position 25. In some embodiments, the amino acid substitution at position 25 is selected from the group consisting of L25E and L25F. In some embodiments, the amino acid substitution at position 25 is L25F. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 79. In some embodiments, the IL-15 polypeptide comprises SEQ ID NO: 79. In some embodiments, the IL-15 polypeptide comprises amino acid substitutions at both position 8 and position 62. In some embodiments, the IL-15 polypeptide is secreted. In some embodiments, the IL-15 polypeptide is membrane bound. In some embodiments, the IL-15 polypeptide comprises a GPI-anchoring peptide sequence. In some embodiments, the IL-15 polypeptide comprises a transmembrane domain. In some embodiments, the IL-15 polypeptide comprises a membrane anchoring domain. In some embodiments, the IL-15 polypeptide is a fusion protein comprising an IL-15 fragment fused to a second polypeptide fragment. In some embodiments, the second polypeptide fragment is selected from the group consisting of IL-15Rα, an extracellular domain of IL-15Rα, a Sushi domain of IL-15Rα, a transmembrane domain of IL-15Rα, IL-15Rβ, common gamma chain (γc), an engineered receptor (e.g., CAR, TCR or TAC) and combinations thereof. In some embodiments, the immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a TAC-expressing immune cell (e.g., TAC-T cell) comprising a heterologous nucleic acid sequence encoding an IL-15 polypeptide comprising one or more amino acid substitutions at positions 8, 62, 3 and/or 25, wherein numbering of the amino acid residue positions is according to SEQ ID NO: 1. In some embodiments, the IL-15 polypeptide comprises an amino acid substitution at position 62. In some embodiments, the amino acid substitution at position 62 is selected from the group consisting of T62G, T62I, T62Q, T62V, T62P, T62L, T62A, T62S and T62Y. In some embodiments, the amino acid substitution at position 62 is T62G. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 7. In some embodiments, the IL-15 polypeptide comprises SEQ ID NO: 7. In some embodiments, the IL-15 polypeptide comprises an amino acid substitution at position 8. In some embodiments, the amino acid substitution at position 8 is D8E. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 5. In some embodiments, the IL-15 polypeptide comprises SEQ ID NO: 5. In some embodiments, the IL-15 polypeptide comprises an amino acid substitution at position 3. In some embodiments, the amino acid substitution at position 3 is V3Y. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 78. In some embodiments, the IL-15 polypeptide comprises SEQ ID NO: 78. In some embodiments, the IL-15 polypeptide comprises an amino acid substitution at position 25. In some embodiments, the amino acid substitution at position 25 is selected from the group consisting of L25E and L25F. In some embodiments, the amino acid substitution at position 25 is L25F. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 79. In some embodiments, the IL-15 polypeptide comprises SEQ ID NO: 79. In some embodiments, the IL-15 polypeptide comprises amino acid substitutions at position 8 and position 62. In some embodiments, the IL-15 polypeptide is secreted. In some embodiments, the IL-15 polypeptide is membrane bound. In some embodiments, the IL-15 polypeptide comprises a GPI-anchoring peptide sequence. In some embodiments, the IL-15 polypeptide comprises a transmembrane domain. In some embodiments, the IL-15 polypeptide comprises a membrane anchoring domain. In some embodiments, the IL-15 polypeptide is a fusion protein comprising an IL-15 fragment fused to a second polypeptide fragment. In some embodiments, the second polypeptide fragment is selected from the group consisting of IL-15Rα, an extracellular domain of IL-15Rα, a Sushi domain of IL-15Rα, a transmembrane domain of IL-15Rα, IL-15Rβ, common gamma chain (γc), an engineered receptor (e.g., CAR, TCR or TAC) and combinations thereof. In some embodiments, the immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a CAR-expressing immune cell (e.g., CAR-NK cell or CAR-T cell) comprising a heterologous nucleic acid sequence encoding an IL-15 polypeptide comprising a T62G substitution, wherein numbering of the amino acid residue positions is according to SEQ ID NO: 1. In some embodiments, the IL-15 polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 7. In some embodiments, the IL-15 polypeptide comprises SEQ ID NO: 7. In some embodiments, the CAR is a BCMA CAR, a CD19 CAR, or a GPC3 CAR. In some embodiments, the immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a CAR-expressing immune cell (e.g., CAR-NK cell or CAR-T cell) comprising a heterologous nucleic acid sequence encoding an IL-15 polypeptide comprising a D8E substitution, wherein numbering of the amino acid residue positions is according to SEQ ID NO: 1. In some embodiments, the IL-15 polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 5. In some embodiments, the IL-15 polypeptide comprises SEQ ID NO: 5. In some embodiments, the CAR is a BCMA CAR, a CD19 CAR, or a GPC3 CAR. In some embodiments, the immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a CAR-expressing immune cell (e.g., CAR-NK cell or CAR-T cell) comprising a heterologous nucleic acid sequence encoding an TL-15 polypeptide comprising a V3Y substitution, wherein numbering of the amino acid residue positions is according to SEQ ID NO: 1. In some embodiments, the IL-15 polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 78. In some embodiments, the IL-15 polypeptide comprises SEQ ID NO: 78. In some embodiments, the CAR is a BCMA CAR, a CD19 CAR, or a GPC3 CAR. In some embodiments, the immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a CAR-expressing immune cell (e.g., CAR-NK cell or CAR-T cell) comprising a heterologous nucleic acid sequence encoding an IL-15 polypeptide comprising a L25F substitution, wherein numbering of the amino acid residue positions is according to SEQ ID NO: 1. In some embodiments, the IL-15 polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 79. In some embodiments, the IL-15 polypeptide comprises SEQ ID NO: 79. In some embodiments, the CAR is a BCMA CAR, a CD19 CAR, or a GPC3 CAR. In some embodiments, the immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a modified immune cell (e.g., NK cell or T cell) comprising a heterologous nucleic acid encoding an IL-15 polypeptide comprising an amino acid sequence selected from the group consisting of SEQ ID NOs: 57-66 and 75-77. In some embodiments, the IL-15 polypeptide is a CAR comprising an IL-15 fragment. In some embodiments, the modified immune cell further comprises a second heterologous nucleic acid encoding a chimeric antigen receptor (CAR). In some embodiments, the CAR is a BCMA CAR, a CD19 CAR, or a GPC3 CAR. In some embodiments, the IL-15 polypeptide is secreted from the modified immune cell. In some embodiments, the IL-15 polypeptide is membrane-bound. In some embodiments, the immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

### Immune Cells

The modified immune cell can be derived from a variety of cell types and cell sources. Cells from any mammalian species, including, but not limited to, mice, rats, guinea pigs, rabbits, dogs, monkeys, and humans, are contemplated herein. In some embodiments, the modified immune cell is a human cell. In some embodiments, the modified immune cell is allogenic (i.e., from the same species, but different donor) as the recipient individual. In some embodiments, the modified immune cell is autologous (i.e., the donor and the recipient are the same). In some embodiments, the modified immune cell is syngeneic (i.e., the donor and the recipients are different individuals, but are identical twins).

In some embodiments, the modified immune cell is derived from a primary cell. In some embodiments, the modified immune cell is a primary cell isolated from an individual. In some embodiments, the modified immune cell is propagated (such as proliferated and/or differentiated) from a primary cell isolated from an individual. In some embodiments, the primary cell is of the hematopoietic lineage. In some embodiments, the primary cell is obtained from the thymus. In some embodiments, the primary cell is obtained from the lymph or lymph nodes (such as tumor draining lymph nodes). In some embodiments, the primary cell is obtained from the spleen. In some embodiments, the primary cell is obtained from the bone marrow. In some embodiments, the primary cell is obtained from the blood, such as the peripheral blood. In some embodiments, the primary cell is a Peripheral Blood Mononuclear Cell (PBMC). In some embodiments, the primary cell is derived from the blood plasma. In some embodiments, the primary cell is derived from a tumor. In some embodiments, the primary cell is obtained from the mucosal immune system. In some embodiments, the primary cell is obtained from a biopsy sample.

In some embodiments, the modified immune cell is derived from a cell line. In some embodiments, the modified immune cell is obtained from a commercial cell line. In some embodiments, the modified immune cell is a cell line established from a primary cell isolated from an individual. In some embodiments, the modified immune cell is propagated (such as proliferated and/or differentiated) from a cell line. In some embodiments, the cell line is mortal. In some embodiments, the cell line is immortalized. In some embodiments, the cell line is a tumor cell line, such as a leukemia or lymphoma cell line. In some embodiments, the cell line is a cell line derived from the PBMC. In some embodiments, the cell line is a stem cell line. In some embodiments, the cell line is selected from the group consisting of HEK293-6E cells, NK-92 cells, and Jurkat cells.

Exemplary immune cells useful for the present application include, but are not limited to, dendritic cells (including immature dendritic cells and mature dendritic cells), T lymphocytes (such as naïve T cells, effector T cells, memory T cells, cytotoxic T lymphocytes, T helper cells, Natural Killer T cells, Treg cells, tumor infiltrating lymphocytes (TIL), and lyphokine-activated killer (LAK) cells), B cells, Natural Killer (NK) cells, monocytes, macrophages, neutrophils, granulocytes, and combinations thereof. Subpopulations of immune cells can be defined by the presence or absence of one or more cell surface markers known in the art (e.g., CD3, CD4, CD8, CD19, CD20, CD11c, CD123, CD56, CD34, CD14, CD33, etc.). In the cases that the pharmaceutical composition comprises a plurality of modified immune cells, the modified immune cells can be a specific subpopulation of an immune cell type, a combination of subpopulations of an immune cell type, or a combination of two or more immune cell types. In some embodiments, the immune cell is present in a homogenous cell population. In some embodiments, the immune cell is present in a heterogeneous cell population that is enhanced in the immune cell. In some embodiments, the modified immune cell is a lymphocyte. In some embodiments, the modified immune cell is not a lymphocyte. In some embodiments, the modified immune cell is suitable for adoptive immunotherapy. In some embodiments, the modified immune cell is a PBMC. In some embodiments, the modified immune cell is an immune cell derived from the PBMC. In some embodiments, the modified immune cell is a T cell. In some embodiments, the modified immune cell is a CD4⁺ T cell. In some embodiments, the modified immune cell is a CD8⁺ T cell. In some embodiments, the modified immune cell is a B cell. In some embodiments, the modified immune cell is an NK cell.

In some embodiments, the modified immune cell is derived from a stem cell. In some embodiments, the stem cell is a totipotent stem cell. In some embodiments, the stem cell is a pluripotent stem cell. In some embodiments, the stem cell is a unipotent stem cell. In some embodiments, the stem cell is a progenitor cell. In some embodiments, the stem cell is an embryonic stem cell. In some embodiments, the stem cell is hematopoietic stem cell. In some embodiments, the stem cell is a mesenchymal stem cell. In some embodiments, the stem cell is an induced pluripotent stem cell (iPSC).

The modified immune cell may comprise any number (such as any of 1, 2, 3, 4, 5, 10, 50, 100, 1000, or more) of the heterologous nucleic acid sequence (including first and second nucleic acid sequences). In some embodiments, the modified immune cell comprises a single copy of the first and/or second heterologous nucleic acid sequence. In some embodiments, the modified immune cell comprises a plurality of copies of the first and/or second heterologous nucleic acid sequence. In some embodiments, the modified immune cell further comprises at least one additional heterologous nucleic acid sequence, for example, a heterologous nucleic acid sequence encoding an immunomodulatory agent, such as cytokine, chemokine, and/or an immune checkpoint inhibitor.

Nucleic acid(s) comprising the heterologous nucleic acid sequence(s) described herein may be transiently or stably incorporated in the modified immune cell. In some embodiments, the nucleic acid(s) is transiently expressed in the modified immune cell. For example, the nucleic acid(s) may be present in the nucleus of the modified immune cell in an extrachromosomal array. The nucleic acid(s) may be introduced into the modified immune cell using any transfection or transduction methods known in the art, including viral or non-viral methods. Exemplary non-viral transfection methods include, but are not limited to, chemical-based transfection, such as using calcium phosphate, dendrimers, liposomes, or cationic polymers (e.g., DEAE-dextran or polyethylenimine); non-chemical methods, such as electroporation, cell squeezing, sonoporation, optical transfection, impalefection, protoplast fusion, hydrodynamic delivery, or transposons; particle-based methods, such as using a gene gun, magnetofection or magnet assisted transfection, particle bombardment; and hybrid methods, such as nucleofection.

In some embodiments, the heterologous nucleic acid sequence(s) is present in the genome of the modified immune cell. For example, nucleic acid(s) comprising the heterologous nucleic acid sequence(s) may be integrated into the genome of the modified immune cell by any methods known in the art, including, but not limited to, virus-mediated integration, random integration, homologous recombination methods, and site-directed integration methods, such as using site-specific recombinase or integrase, transposase, Transcription activator-like effector nuclease (TALEN®), CRISPR/Cas9, and zinc-finger nucleases. In some embodiments, the heterologous nucleic acid sequence(s) is integrated in a specifically designed locus of the genome of the modified immune cell. In some embodiments, the heterologous nucleic acid sequence(s) is integrated in an integration hotspot of the genome of the modified immune cell. In some embodiments, the heterologous nucleic acid (sequence) is integrated in a random locus of the genome of the modified immune cell. In the cases that multiple copies of the heterologous nucleic acid sequence(s) are present in a single modified immune cell, the heterologous nucleic acid sequences may be integrated in a plurality of loci of the genome of the modified immune cell.

### IL-15 Polypeptide

The modified immune cells described herein express a mutant IL-15 polypeptide. The present application also provides IL-15 polypeptides and compositions thereof. In some embodiments, the IL-15 polypeptides provided herein provide strong anti-tumor effects without producing increased levels of inflammatory cytokines (e.g., a cytokine storm).

In some embodiments, the IL-15 polypeptide is a full-length IL-15 molecule. In some embodiments, the IL-15 polypeptide comprises a functional portion of an IL-15 molecule. In some embodiments, the IL-15 polypeptide is a human IL-15 polypeptide. In some embodiments, the IL-15 polypeptide has a single chain. In some embodiments, the IL-15 polypeptide has two or more chains.

In some embodiments, the IL-15 polypeptides described herein are capable of binding to a trimeric IL-15R (IL-15 receptor) complex. The IL-15 receptor consists of three polypeptides, the type-specific IL-15 (“IL-15Rα”), the IL-2/IL-15Rβ (“IL-15Rβ”), and the common gamma chain (“γc”) shared by various cytokines. In some embodiments, the IL-15 polypeptide is capable of binding the alpha-chain (“IL-15Rα”), the common beta-chain (“IL-15Rβ”), and/or the common gamma-chain (“IL-15Rγc”). In some embodiments, the IL-15 polypeptide is capable of binding IL-15Rα. In some embodiments, the IL-15 polypeptide is capable of binding IL-15Rβ. In some embodiments, the IL-15 polypeptide is capable of binding IL-15Rβ/γc.

In some embodiments, the IL-15 polypeptide has comparable binding affinity to IL-15Rα as a wildtype IL-15 polypeptide. In some embodiments, the IL-15 polypeptide has reduced binding affinity to IL-15β compared to a wildtype IL-15 polypeptide. An exemplary wildtype IL-15 polypeptide has the amino acids sequence of SEQ ID NO: 1.

Wildtype human IL-15
SEQ ID NO: 1
NWVNVISDLKKIEDLIQSMHIDATLYTESDVHPSCKVTAMKCFLLELQV
ISLESGDASIHDTVENLIILANNSLSSNGNVTESGCKECEELEEKNIKE
FLQSFVHIVQMFINTS

In some embodiments, the IL-15 polypeptide binds to IL-15Rα with a KD of no lower than about any one of 10⁻⁹, 10⁻¹⁰, or 10⁻¹². In some embodiments, the IL-15 polypeptide binds to IL-15Rβ with a KD of no lower than about any one of 10⁻⁷, 10⁻⁸, or 10⁻⁹.

In some embodiments, the IL-15 polypeptide has reduced binding affinity, such as reduced by at least about any one of 10%, 20%, 30%, 40%, 2 fold, 4 fold, 6 fold, 8 fold, 10 fold, 12 fold, 14 fold, 16 fold, 18 fold, 20 fold, 30 fold, 50 fold, or more, to IL-15Rβ compared to a wildtype IL-15 polypeptide. In some embodiments, the IL-15 polypeptide has reduced binding affinity, such as reduced by no more than about any one of 50 fold, 30 fold, 20 fold, 18 fold, 16 fold, 14 fold, 12 fold, 10 fold, 8 fold, 6 fold, 4 fold, 50%, 40%, 30%, 20%, 10% or less, to IL-15Rβ compared to a wildtype IL-15 polypeptide. In some embodiments, the IL-15 polypeptide has reduced binding affinity, such as reduced by between about any of 10%-50%, 2-10 fold, 10-20 fold, 20-40 fold, 10-40 fold, 10-50 fold, 14-40 fold, or 2-50 fold, to IL-15Rβ compared to a wildtype IL-15 polypeptide.

In some embodiments, the IL-15 polypeptide induces a reduced level of inflammatory cytokine secretion by the modified immune cell, such as reduced by at least about any one of 10%, 20%, 30%, 40%, 2 fold, 4 fold, 6 fold, 8 fold, 10 fold, 12 fold, 14 fold, 16 fold, 18 fold, 20 fold, 30 fold, 50 fold, 100 fold, 200 fold, 500 fold, 1000 fold, or more, compared to a wildtype IL-15 polypeptide. In some embodiments, the IL-15 polypeptide induces a reduced level of inflammatory cytokine secretion by the modified immune cell, such as reduced by no more than about any one of 1000 fold, 500 fold, 200 fold, 100 fold, 50 fold, 30 fold, 20 fold, 18 fold, 16 fold, 14 fold, 12 fold, 10 fold, 8 fold, 6, 4 fold fold, 50%, 40%, 30%, 20%, 10% or less, compared to a wildtype IL-15 polypeptide. In some embodiments, the IL-15 polypeptide induces a reduced level of inflammatory cytokine secretion by the modified immune cell, such as reduced by between about any of 10%-50%, 2-1000 fold, 2-50 fold, 50-100 fold, 100-1000 fold, 50-500 fold, 10-100 fold, 10-50 fold, or 50-200 fold, compared to a wildtype IL-15 polypeptide. Exemplary inflammatory cytokines include, but are not limited to, e.g., IFN-γ, TNF-α, and GM-CSF. In some embodiments, secretion levels of inflammatory cytokines are measured by in serum immunoassays, such as enzyme-linked immunosorbent assay (ELISA), chemiluminescent immunoassays (CIA), or flow cytometry. In some embodiments, the inflammatory cytokine secretion levels are measured in a cell-based assay. In some embodiments, the inflammatory cytokine secretion levels are measured in vivo.

In some embodiments, the IL-ae polypeptide does not induce a cytokine storm in a subject receiving the IL-15 polypeptide, or a modified immune cell comprising the IL-15 polypeptide. A “cytokine storm” occurs when numerous proinflammatory cytokines are generated at a higher rate than normal. In some embodiments, the overproduction of cytokines may cause cellular damage due to the recruitment of other immune cells. In some embodiments, the proinflammatory cytokines produced during a cytokine storm include members of the IL-20 family, IL-1-α, IL1-β, IL-6, IL-33 LIF, IFN-γ, OSM, CNTF, TNF-α, TGF-β, GM-CSF, IL-11, IL-12, IL-17, IL-18, IL-8, and other proinflammatory cytokines known in the art. In some embodiments, the proinflammatory cytokines produced during a cytokine storm include IFN-γ, TNF-α, and GM-CSF. In some embodiments, the cytokine storm may be measured by any cytokine measuring technique known in the art. In some embodiments, the cytokine storm is measured by ELISA, CIA, or flow cytometry.

In some embodiments, the IL-15 polypeptide comprises an amino acid substitution at one or more positions selected from the group consisting of 3, 8, 23, 25, 26, 58, 61, 62, and 89, wherein numbering of the amino acid residue positions is according to SEQ TD NO: 1. Exemplary mutant IL-15 polypeptide sequences and their corresponding mutations are listed in Table 1 below.

TABLE 1
Exemplary mutant IL-15 polypeptides (also referred herein as IL-15 muteins)

| | | | SEQ |
|---|---|---|---|
| | | | ID |
| Name | Mutation | Amino acid sequence | NO. |
| mutein 1 | A23L | NWVNVISDLKKIEDLIQSMHIDLTLYTESDVHPSCKVTAMK | 2 |
| (m1) | | CFLLELQVISLESGDASIHDTVENLIILANNSLSSNGNVTESG | |
| | | CKECEELEEKNIKEFLQSFVHIVQMFINTS | |
| mutein 2 | L25E | NWVNVISDLKKIEDLIQSMHIDATEYTESDVHPSCKVTAMK | 3 |
| (m2) | | CFLLELQVISLESGDASIHDTVENLIILANNSLSSNGNVTESG | |
| | | CKECEELEEKNIKEFLQSFVHIVQMFINTS | |
| mutein 3 | Y26G | NWVNVISDLKKIEDLIQSMHIDATLGTESDVHPSCKVTAMK | 4 |
| (m3) | | CFLLELQVISLESGDASIHDTVENLIILANNSLSSNGNVTESG | |
| | | CKECEELEEKNIKEFLQSFVHIVQMFINTS | |
| mutein 4 | D8E | NWVNVISELKKIEDLIQSMHIDATLYTESDVHPSCKVTAMK | 5 |
| (m4) | | CFLLELQVISLESGDASIHDTVENLIILANNSLSSNGNVTESG | |
| | | CKECEELEEKNIKEFLQSFVHIVQMFINTS | |
| mutein 5 | D61E | NWVNVISDLKKIEDLIQSMHIDATLYTESDVHPSCKVTAMK | 6 |
| (m5) | | CFLLELQVISLESGDASIHETVENLIILANNSLSSNGNVTESG | |
| | | CKECEELEEKNIKEFLQSFVHIVQMFINTS | |
| mutein 6 | T62G | NWVNVISDLKKIEDLIQSMHIDATLYTESDVHPSCKVTAMK | 7 |
| (m6) | | CFLLELQVISLESGDASIHDGVENLIILANNSLSSNGNVTESG | |
| | | CKECEELEEKNIKEFLQSFVHIVQMFINTS | |
| mutein 7 | T62I | NWVNVISDLKKIEDLIQSMHIDATLYTESDVHPSCKVTAMK | 8 |
| (m7) | | CFLLELQVISLESGDASIHDIVENLIILANNSLSSNGNVTESG | |
| | | CKECEELEEKNIKEFLQSFVHIVQMFINTS | |
| mutein 8 | E89K | NWVNVISDLKKIEDLIQSMHIDATLYTESDVHPSCKVTAMK | 9 |
| (m8) | | CFLLELQVISLESGDASIHDTVENLIILANNSLSSNGNVTESG | |
| | | CKECKELEEKNIKEFLQSFVHIVQMFINTS | |
| mutein 9 | S58D | NWVNVISDLKKIEDLIQSMHIDATLYTESDVHPSCKVTAMK | 10 |
| (m9) | | CFLLELQVISLESGDADIHDTVENLIILANNSLSSNGNVTESG | |
| | | CKECEELEEKNIKEFLQSFVHIVQMFINTS | |
| mutein | T62Q | NWVNVISDLKKIEDLIQSMHIDATLYTESDVHPSCKVTAMK | 11 |
| 10 | | CFLLELQVISLESGDASIHDQVENLIILANNSLSSNGNVTESG | |
| (m10) | | CKECEELEEKNIKEFLQSFVHIVQMFINTS | |
| mutein | T62V | NWVNVISDLKKIEDLIQSMHIDATLYTESDVHPSCKVTAMK | 12 |
| 11 | | CFLLELQVISLESGDASIHDVVENLIILANNSLSSNGNVTESG | |
| (m11) | | CKECEELEEKNIKEFLQSFVHIVQMFINTS | |
| mutein | T62P | NWVNVISDLKKIEDLIQSMHIDATLYTESDVHPSCKVTAMK | 13 |
| 12 | | CFLLELQVISLESGDASIHDPVENLIILANNSLSSNGNVTESG | |
| (m12) | | CKECEELEEKNIKEFLQSFVHIVQMFINTS | |
| mutein | T62L | NWVNVISDLKKIEDLIQSMHIDATLYTESDVHPSCKVTAMK | 14 |
| 13 | | CFLLELQVISLESGDASIHDLVENLIILANNSLSSNGNVTESG | |
| (m13) | | CKECEELEEKNIKEFLQSFVHIVQMFINTS | |
| mutein | T62A | NWVNVISDLKKIEDLIQSMHIDATLYTESDVHPSCKVTAMK | 15 |
| 14 | | CFLLELQVISLESGDASIHDAVENLIILANNSLSSNGNVTESG | |
| (m14) | | CKECEELEEKNIKEFLQSFVHIVQMFINTS | |
| mutein | T62S | NWVNVISDLKKIEDLIQSMHIDATLYTESDVHPSCKVTAMK | 16 |
| 15 | | CFLLELQVISLESGDASIHDSVENLIILANNSLSSNGNVTESG | |
| (m15) | | CKECEELEEKNIKEFLQSFVHIVQMFINTS | |
| mutein | T62Y | NWVNVISDLKKIEDLIQSMHIDATLYTESDVHPSCKVTAMK | 17 |
| 16 | | CFLLELQVISLESGDASIHDYVENLIILANNSLSSNGNVTESG | |
| (m16) | | CKECEELEEKNIKEFLQSFVHIVQMFINTS | |
| mutein | V3Y | NWYNVISDLKKIEDLIQSMHIDATLYTESDVHPSCKVTAMK | 78 |
| 17 | | CFLLELQVISLESGDASIHDTVENLIILANNSLSSNGNVTESG | |
| (m17) | | CKECEELEEKNIKEFLQSFVHIVQMFINTS | |
| mutein | L25F | NWVNVISDLKKIEDLIQSMHIDATFYTESDVHPSCKVTAMK | 79 |
| 18 | | CFLLELQVISLESGDASIHDTVENLIILANNSLSSNGNVTESG | |
| (m18) | | CKECEELEEKNIKEFLQSFVHIVQMFINTS | |

In some embodiments, the IL-15 polypeptide comprises an amino acid substitution at position 62, wherein numbering of the amino acid residue positions is according to SEQ ID NO: 1. In some embodiments, the IL-15 polypeptide comprises a hydrophobic amino acid residue at position 62. In some embodiments, the IL-15 polypeptide comprises an amino acid residue having a short side chain at position 62. In some embodiments, the IL-15 polypeptide comprises an amino acid residue selected from the group consisting of Glycine (G), Isoleucine (I), Glutamine (Q), Valine (V), Proline (P), Leucine (L), Alanine (A), Serine (S) and Tyrosine (Y) at position 62. In some embodiments, the IL-15 polypeptide comprises an amino acid substitution selected from the group consisting of T62G, T62I, T62Q, T62V, T62P, T62L, T62A, T62S and T62Y. In some embodiments, the IL-15 polypeptide comprises a T62G substitution. In some embodiments, the IL-15 polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to an amino acid sequence selected from the group consisting of SEQ ID NOs: 7, 8 and 11-17. In some embodiments, the IL-15 polypeptide comprises an amino acid sequence selected from the group consisting of SEQ ID NOs: 7, 8 and 11-17. In some embodiments, the IL-15 polypeptide comprises a single amino acid substitution described herein. In some embodiments, the IL-15 polypeptide comprises two or more amino acid substitutions described here.

In some embodiments, the IL-15 polypeptide comprises an amino acid substitution at position 8, wherein numbering of the amino acid residue positions is according to SEQ ID NO: 1. In some embodiments, the IL-15 polypeptide comprises an acidic amino acid residue at position 8. In some embodiments, the IL-15 polypeptide comprises an E at position 8. In some embodiments, the IL-15 polypeptide comprises a non-charged amino acid residue at position 8. In some embodiments, the IL-15 polypeptide comprises a D8E substitution. In some embodiments, the IL-15 polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 5. In some embodiments, the IL-15 polypeptide comprises the amino acid sequence of SEQ ID NO: 5. In some embodiments, the IL-15 polypeptide comprises a single amino acid substitution described herein. In some embodiments, the IL-15 polypeptide comprises two or more amino acid substitutions described here. In some embodiments, the IL-15 polypeptide comprises an amino acid substitution at position 8 and an amino acid substitution at position 62. In some embodiments, the IL-15 polypeptide comprises D8E and T62G substations.

In some embodiments, the IL-15 polypeptide comprises an amino acid substitution at position 3, wherein numbering of the amino acid residue positions is according to SEQ ID NO: 1. In some embodiments, the IL-15 polypeptide comprises an Y at position 3. In some embodiments, the IL-15 polypeptide comprises a V3Y substitution. In some embodiments, the IL-15 polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 78. In some embodiments, the IL-15 polypeptide comprises the amino acid sequence of SEQ ID NO: 78. In some embodiments, the IL-15 polypeptide comprises a single amino acid substitution described herein. In some embodiments, the IL-15 polypeptide comprises two or more amino acid substitutions described here.

In some embodiments, the IL-15 polypeptide comprises an amino acid substitution at position 25, wherein numbering of the amino acid residue positions is according to SEQ ID NO: 1. In some embodiments, the IL-15 polypeptide comprises an F at position 25. In some embodiments, the IL-15 polypeptide comprises a L25F substitution. In some embodiments, the IL-15 polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 79. In some embodiments, the IL-15 polypeptide comprises the amino acid sequence of SEQ ID NO: 79. In some embodiments, the IL-15 polypeptide comprises a single amino acid substitution described herein. In some embodiments, the IL-15 polypeptide comprises two or more amino acid substitutions described here.

In some embodiments, the IL-15 polypeptide comprises amino acid substitutions at position 8 and position 62. In some embodiments, the IL-15 polypeptide comprises D8E and T62G substations. In some embodiments, the IL-15 polypeptide comprises amino acid substitutions at position 8 and position 3. In some embodiments, the IL-15 polypeptide comprises D8E and V3Y substations. In some embodiments, the IL-15 polypeptide comprises amino acid substitutions at position 8 and position 25. In some embodiments, the IL-15 polypeptide comprises D8E and L25F substations. In some embodiments, the IL-15 polypeptide comprises amino acid substitutions at position 62 and position 3. In some embodiments, the IL-15 polypeptide comprises T62G and V3Y substations. In some embodiments, the IL-15 polypeptide comprises amino acid substitutions at position 62 and position 25. In some embodiments, the IL-15 polypeptide comprises T62G and L25F substations. In some embodiments, the IL-15 polypeptide comprises amino acid substitutions at position 3 and position 25. In some embodiments, the IL-15 polypeptide comprises V3Y and L25F substations. In some embodiments, the IL-15 polypeptide comprises amino acid substitutions at position 8, position 62 and position 3. In some embodiments, the IL-15 polypeptide comprises D8E, T62G and V3Y substations. In some embodiments, the IL-15 polypeptide comprises amino acid substitutions at position 8, position 62 and position 25. In some embodiments, the IL-15 polypeptide comprises D8E, T62G and L25F substations. In some embodiments, the IL-15 polypeptide comprises amino acid substitutions at position 8, position 3 and position 25. In some embodiments, the IL-15 polypeptide comprises D8E, V3Y and L25F substations. In some embodiments, the IL-15 polypeptide comprises amino acid substitutions at position 62, position 3 and position 25. In some embodiments, the IL-15 polypeptide comprises T62G, V3Y and L25F substations. In some embodiments, the IL-15 polypeptide comprises amino acid substitutions at position 8, position 62, position 3 and position 25. In some embodiments, the IL-15 polypeptide comprises D8E, T62G, V3Y and L25F substations.

In some embodiments, the IL-15 polypeptide is secreted from the modified immune cell. In some embodiments, the IL-15 polypeptide comprises an IL-15 fragment fused to an extracellular domain of IL-15Rα. In some embodiments, the IL-15 polypeptide comprises an IL-15 fragment fused to a Sushi domain of IL-15Rα. In some embodiments, the IL-15 polypeptide comprises a signal peptide (also referred herein as “SP”). The signal peptide (also known as “leader sequence”) is typically inserted at the N-terminus of the protein immediately after the Met initiator. Signal peptides may be cleaved upon export of the IL-15 polypeptide from the modified immune cell, forming a mature protein. Signal peptides may be natural or synthetic, and they may be heterologous or homologous to the protein to which they are attached. The choice of signal peptides is wide and is accessible to persons skilled in the art, including, for example, in the online Leader sequence Database maintained by the Department of Biochemistry, National University of Singapore. See Choo et al., BMC Bioinformatics, 6: 249 (2005); and PCT Publication No. WO 2006/081430. Exemplary signal peptide sequences include, but are not limited to SEQ ID NOs: 71-74. In some embodiments, the IL-15 polypeptide comprises a pro-peptide. In some embodiments, the IL-15 polypeptide comprises SEQ ID NO: 70.

In some embodiments, the IL-15 polypeptide is a fusion protein comprising an IL-15 fragment fused to a second polypeptide fragment. In some embodiments, the second polypeptide fragment is selected from the group consisting of IL-15Rα, an extracellular domain of IL-15Rα, a Sushi domain of IL-15Rα, a transmembrane domain of IL-15Rα, IL-15Rβ, common gamma chain (γc), an engineered receptor (e.g., CAR, TCR or TAC) and combinations thereof.

Exemplary IL-15 fusion polypeptides are listed in Table 2 below. It is understood that fusion proteins comprising any one of the IL-15 muteins described herein are contemplated herein.

TABLE 2
Exemplary fusion IL-15 polypeptides

| | Structure (from N-terminus | |
|---|---|---|
| Name | to C-terminus) | SEQ ID NO |
| sIL-15 Rα | e.g., SP-m6-linker-IL15Rα | 57 |
| | Extracellular Domain (ED) | |
| sIL-15 sRα | e.g., SP-m6-linker-IL15Rα Sushi domain | 58 |
| mb-1 IL-15 | e.g., SP-m6-linker-IL-15Rα | 59 |
| | Sushi domain-linker- | |
| | IL15RαTM | |
| mb-2 IL-15 | e.g., SP-m6-linker - VHH1- | 65 |
| armored CAR | VHH2-CD8αTM-4-1BB - | |
| | CD3zeta | |
| mb-3 IL-15 | e.g., CD8αSP-VHH1-VHH2-linker- | 66 |
| armored CAR | m6-linker-CD8αTM- | |
| | 4-1BB -CD3zeta | |
| mb-4 IL-15 | e.g., SP-m6- linker -IL-15Rβ - | 60 |
| | self-cleaving peptide - γc | |
| mb-5 IL-15 | e.g., SP-m6 - linker -IL-15Rα | 61 |
| mb-6 IL-15 | e.g., SP-m6 - linker-IL-15Rβ | 62 |
| mb-7 IL-15 | e.g., SP-m6 - linker - γc | 63 |
| mb-8 IL-15 | e.g., SP-m6 -linker-IL-15Rα TM | 64 |

In some embodiments, the IL-15 polypeptide is a fusion protein comprising an IL-15 fragment fused to an IL-15 receptor (IL-15R), a subunit thereof or a portion thereof. In some embodiments, the IL-15 polypeptide comprises an IL-15 fragment fused to IL-15Rα. In some embodiments, the IL-15Rα is a full-length IL-15Rα molecule. In some embodiments, the IL-15Rα is a soluble form of IL-15Rα (e.g., sIL-15R or sIL-15Rα). In some embodiments, the IL-15Rα is an extracellular domain of a naturally occurring IL-15Rα molecule. In some embodiments, the IL-15R comprises truncated or deleted cytoplasmic and transmembrane domains but retains functional domains (e.g., regions of IL-15R required to retain IL-15 binding activity). In some embodiments, the shortest region of IL-15R retaining IL-15 binding activity is a 65 amino acid sequence spanning the Sushi domain of IL-15Rα. “Sushi domains”, or “short consensus repeats” or “type 1 glycoprotein motifs”, are common structural motifs that facilitate protein-protein interactions, wherein the motif comprises four cysteines that form two disulfide bonds. In some embodiments, the IL-15Rα is a Sushi domain of naturally occurring IL-15Rα molecule. In some embodiments, the IL-15 polypeptide comprises an IL-15 fragment fused to a transmembrane domain of IL-15Rα. In some embodiments, the IL-15 polypeptide comprises an IL-15 fragment fused to a Sushi domain and a transmembrane domain of IL-15Rα.

Human IL-15Rα and its fragments are shown below.

(IL-15Rα extracellular domain)
SEQ ID NO: 50
ITCPPPMSVEHADIWVKSYSLYSRERYICNSGFKRKAGTSSLTECVLNK
ATNVAHWTTPSLKCIRDPALVHQRPAPPSTVTTAGVTPQPESLSPSGKE
PAASSPSSNNTAATTAAIVPGSQLMPSKSPSTGTTEISSHESSHGTPSQ
TTAKNWELTASASHQPPGVYPQGHSDTT
(IL-15Rα Sushi domain)
SEQ ID NO: 51
ITCPPPMSVEHADIWVKSYSLYSRERYICNSGFKRKAGTSSLTECVLNK
ATNVAHWTTPSLKCIR
(full-length IL-15Rα)
SEQ ID NO: 54
ITCPPPMSVEHADIWVKSYSLYSRERYICNSGFKRKAGTSSLTECVLNK
ATNVAHWTTPSLKCIRDPALVHQRPAPPSTVTTAGVTPQPESLSPSGKE
PAASSPSSNNTAATTAAIVPGSQLMPSKSPSTGTTEISSHESSHGTPSQ
TTAKNWELTASASHQPPGVYPQGHSDTTVAISTSTVLLCGLSAVSLLAC
YLKSRQTPPLASVEMEAMEALPVTWGTSSRDEDLENCSHHL
(IL-15Rα transmembrane domain)
SEQ ID NO: 55
VAISTSTVLLCGLSAVSLLACYL

In some embodiments, the IL-15 polypeptide comprises an IL-15 fragment fused to IL-15Rβ. In some embodiments, the IL-15Rβ is a full-length IL-15Rβ molecule. In some embodiments, the IL-15Rβ comprises the amino acid sequence of SEQ ID NO: 52.

(full-length IL15Rβ)
SEQ ID NO: 52
AVNGTSQFTCFYNSRANISCVWSQDGALQDTSCQVHAWPDRRRWNQTCE
LLPVSQASWACNLILGAPDSQKLTTVDIVTLRVLCREGVRWRVMAIQDF
KPFENLRLMAPISLQVVHVETHRCNISWEISQASHYFERHLEFEARTLS
PGHTWEEAPLLTLKQKQEWICLETLTPDTQYEFQVRVKPLQGEFTTWSP
WSQPLAFRTKPAALGKDTIPWLGHLLVGLSGAFGFIILVYLLINCRNTG
PWLKKVLKCNTPDPSKFFSQLSSEHGGDVQKWLSSPFPSSSFSPGGLAP
EISPLEVLERDKVTQLLLQQDKVPEPASLSSNHSLTSCFTNQGYFFFHL
PDALEIEACQVYFTYDPYSEEDPDEGVAGAPTGSSPQPLQPLSGEDDAY
CTFPSRDDLLLFSPSLLGGPSPPSTAPGGSGAGEERMPPSLQERVPRDW
DPQPLGPPTPGVPDLVDFQPPPELVLREAGEEVPDAGPREGVSFPWSRP
PGQGEFRALNARLPLNTDAYLSLQELQGQDPTHLV

In some embodiments, the IL-15 polypeptide an IL-15 fragment fused to γc. In some embodiments, the γc is a full-length γc molecule. In some embodiments, the γc comprises the amino acid sequence of SEQ ID NO: 53. In some embodiments, the IL-15 polypeptide comprises an IL-15 fragment fused to IL-15Rβ and the modified immune cell further comprises a heterologous nucleic acid sequence encoding γc.

(γc, common gamma chain)
SEQ ID NO: 53
MLKPSLPFTSLLFLQLPLLGVGLNTTILTPNGNEDTTADFFLTTMPTDS
LSVSTLPLPEVQCFVFNVEYMNCTWNSSSEPQPTNLTLHYWYKNSDNDK
VQKCSHYLFSEEITSGCQLQKKEIHLYQTFVVQLQDPREPRRQATQMLK
LQNLVIPWAPENLTLHKLSESQLELNWNNRFLNHCLEHLVQYRTDWDHS
WTEQSVDYRHKFSLPSVDGQKRYTFRVRSRFNPLCGSAQHWSEWSHPIH
WGSNTSKENPFLFALEAVVISVGSMGLIISLLCVYFWLERTMPRIPTLK
NLEDLVTEYHGNFSAWSGVSKGLAESLQPDYSERLCLVSEIPPKGGALG
EGPGASPCNQHSPYWAPPCYTLKPET

In some embodiments, the IL-15 polypeptide is membrane-bound. In some embodiments, the IL-15 polypeptide comprises a glycosylphosphatidylinositol (GPI)—anchoring peptide sequence. In some embodiments, the IL-15 polypeptide comprises a GPI-anchoring polypeptide sequence at the C-terminus. GPI-anchoring polypeptide sequences are known in the art, including, but not limited to the GPI anchor sequence of human LFA3, CD44, CD59, human Fcγ receptor III (CD16b). See Kueng et al., J Virol, 2007, 81(16):8666-8676. In some embodiments, the GPI-anchoring peptide sequence is attached to a GPI linker.

In some embodiments, the IL-15 polypeptide comprises an IL-15 fragment fused to a membrane-anchoring domain. In some embodiments, the membrane-anchoring domain comprises a sequence that can be inserted into a phospholipid bilayer (e.g., amino acid residues with hydrophobic side chains that interact with fatty acyl groups of the membrane phospholipids). In some embodiments, the membrane-anchoring domain comprises a positively charged amino acid sequence. In some embodiments, the membrane-anchoring domain comprises a lipid.

In some embodiments, the IL-15 polypeptide comprises a transmembrane domain that can be directly or indirectly fused to an IL-15 fragment. The transmembrane domain may be derived either from a natural or from a synthetic source. As used herein, a “transmembrane domain” refers to any protein structure that is thermodynamically stable in a cell membrane, preferably a eukaryotic cell membrane. Transmembrane domains compatible for use in the IL-15 polypeptide described herein may be obtained from a naturally occurring protein. Alternatively, it can be a synthetic, non-naturally occurring protein segment, e.g., a hydrophobic protein segment that is thermodynamically stable in a cell membrane.

Transmembrane domains are classified based on the three dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times). Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-termini.

In some embodiments, the transmembrane domain of the IL-15 polypeptide described herein is derived from a Type I single-pass membrane protein. In some embodiments, transmembrane domains from multi-pass membrane proteins may also be compatible for use in the IL-15 polypeptide described herein. Multi-pass membrane proteins may comprise a complex (at least 2, 3, 4, 5, 6, 7 or more) alpha helices or a beta sheet structure. Preferably, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side.

In some embodiments, the transmembrane domain of the IL-15 polypeptide comprises a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL-2R beta, IL-2R gamma, IL-7R a, ITGA1, VLA1, CD49a, ITGA4, IA4, CD49D, ITGA6, VLA-6, CD49f, ITGAD, CD11 d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Lyl08), SLAM (SLAMF1, CD150, IPO-3), BLAME (SLAMF8), SELPLG (CD162), LTBR, PAG/Cbp, NKp44, NKp30, NKp46, NKG2D, and/or NKG2C. In some embodiments, the transmembrane domain is derived from a molecule selected from the group consisting of CD8a, CD4, CD28, 4-1BB, CD80, CD86, CD152 and PD1. In some embodiments, the transmembrane domain is derived from CD8a. In some embodiments, the transmembrane domain is derived from IL-15Rα.

Transmembrane domains for use in the IL-15 polypeptide described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 B1 and PCT Publication No. WO 2000/032776 A2, the relevant disclosures of which are incorporated by reference herein.

The transmembrane domain may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane domain of the IL-15 polypeptide comprises an artificial hydrophobic sequence. For example, a triplet of phenylalanine, tryptophan and valine may be present at the C terminus of the transmembrane domain. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence. The hydropathy, or hydrophobic or hydrophilic characteristics of a protein or protein segment, can be assessed by any method known in the art, for example the Kyte and Doolittle hydropathy analysis.

The IL-15 polypeptide may comprise a hinge region that is located between the IL-15 fragment and the transmembrane domain. A hinge region is an amino acid segment that is generally found between two domains of a protein and may allow for flexibility of the protein and movement of one or both of the domains relative to one another. Any amino acid sequence that provides such flexibility and movement of the IL-15 fragment relative to the transmembrane domain in the IL-15 polypeptide can be used.

The hinge region may contain about 10-100 amino acids, e.g., about any one of 15-75 amino acids, 20-50 amino acids, or 30-60 amino acids. In some embodiments, the hinge region may be at least about any one of 10, 11, 12, 13, 14, 15, 16, 17, 18, 19, 20, 21, 22, 23, 24, 25, 26, 27, 28, 29, 30, 35, 40, 45, 50, 55, 60, 65, 70, or 75 amino acids in length.

In some embodiments, the hinge region is a hinge region of a naturally occurring protein. Hinge regions of any protein known in the art to comprise a hinge region are compatible for use in the IL-15 polypeptides described herein. In some embodiments, the hinge region is at least a portion of a hinge region of a naturally occurring protein and confers flexibility to the IL-15 polypeptide. In some embodiments, the hinge region is derived from CD8α. In some embodiments, the hinge region is a portion of the hinge region of CD8α, e.g., a fragment containing at least 15 (e.g., 20, 25, 30, 35, or 40) consecutive amino acids of the hinge region of CD8α.

Hinge regions of antibodies, such as an IgG, IgA, IgM, IgE, or IgD antibodies, are also compatible for use in the IL-15 polypeptide described herein. In some embodiments, the hinge region is the hinge region that joins the constant domains CH1 and CH2 of an antibody. In some embodiments, the hinge region is of an antibody and comprises the hinge region of the antibody and one or more constant regions of the antibody. In some embodiments, the hinge region comprises the hinge region of an antibody and the CH3 constant region of the antibody. In some embodiments, the hinge region comprises the hinge region of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

Non-naturally occurring peptides may also be used as hinge regions for the IL-15 polypeptide. In some embodiments, the hinge region is a peptide linker, such as a (GxS)n linker, wherein x and n, independently can be an integer between 3 and 12, including 3, 4, 5, 6, 7, 8, 9, 10, 11, 12, or more.

In some embodiments, the IL-15 polypeptide further comprises an intracellular domain, such as an intracellular signaling domain. In some embodiments, the IL-15 polypeptide comprises: (a) an IL-15 fragment, (b) a transmembrane domain; and (c) an intracellular domain.

In some embodiments, the IL-15 polypeptide is an engineered receptor, comprising: (a) an antigen-binding domain; (b) an IL-15 fragment; (c) a transmembrane domain; and (d) an intracellular domain. In some embodiments, the IL-15 polypeptide comprises two or more antigen-binding domains. In some embodiments, the IL-15 polypeptide is a monospecific engineered receptor. In some embodiments, the IL-15 polypeptide is a bispecific engineered receptor. In some embodiments, the IL-15 polypeptide is a multispecific engineered receptor. In some embodiments, the IL-15 polypeptide is a multivalent, such as bi-valent engineered receptor. In some embodiments, the IL-15 polypeptide is a bi-epitope engineered receptor. The antigen-binding domains may be at the N-terminus or the C-terminus of the IL-15 fragment. In some embodiments, the antigen-binding domain is fused to the IL-15 fragment via a peptide linker. Exemplary engineered receptors include, but are not limited to, CAR, TCR, and TAC. The IL-15 polypeptide may include any components of an engineered receptor as described in the subsection “Engineered receptor” below.

In some embodiments, the intracellular domain comprises a co-stimulatory signaling domain. The term “co-stimulatory signaling domain,” as used herein, refers to at least a portion of a protein that mediates signal transduction within a cell to induce an immune response such as an effector function. The co-stimulatory signaling domain of the IL-15 polypeptide described herein can be a cytoplasmic signaling domain from a co-stimulatory protein, which transduces a signal and modulates responses mediated by immune cells, such as T cells, NK cells, DCs, lymph node (LN) stromal cells, macrophages, neutrophils, or eosinophils. “Co-stimulatory signaling domain” can be the cytoplasmic portion of a co-stimulatory molecule. The term “co-stimulatory molecule” refers to a cognate binding partner on an immune cell (such as T cell) that specifically binds with a co-stimulatory ligand, thereby mediating a co-stimulatory response by the immune cell, such as, but not limited to, proliferation and survival.

In some embodiments, the intracellular domain comprises a single co-stimulatory signaling domain. In some embodiments, the intracellular domain comprises two or more (such as about any of 2, 3, 4, or more) co-stimulatory signaling domains. In some embodiments, the intracellular domain comprises two or more of the same co-stimulatory signaling domains, for example, two copies of the co-stimulatory signaling domain of CD28. In some embodiments, the intracellular domain comprises two or more co-stimulatory signaling domains from different co-stimulatory proteins, such as any two or more co-stimulatory proteins described herein. In some embodiments, the one or more co-stimulatory signaling domains are fused to each other via optional peptide linkers. The one or more co-stimulatory signaling domains may be arranged in any suitable order. Multiple co-stimulatory signaling domains may provide additive or synergistic stimulatory effects.

Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The type(s) of co-stimulatory signaling domain is selected based on factors such as the type of the immune cells in which the IL-15 polypeptide would be expressed (e.g., T cells, NK cells, DCs, stromal cells, macrophages, neutrophils, or eosinophils) and the desired immune effector function. Examples of co-stimulatory signaling domains for use in the TL-15 polypeptides can be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACI/TNFRSF13B, TL1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C.

In some embodiments, the one or more co-stimulatory signaling domains are selected from the group consisting of CD27, CD28, 4-1BB (i.e., CD137), OX40, DAP10, CD30, CD40, CD3, lymphocyte function-associated antigen-1(LFA-1), CD2, CD7, LIGHT, NKG2C, B7-H3 and ligands that specially bind to CD83.

In some embodiments, the intracellular domain in the IL-15 polypeptide comprises a co-stimulatory signaling domain derived from CD28. In some embodiments, the intracellular domain in the IL-15 polypeptide comprises a co-stimulatory signaling domain derived from 4-1BB (i.e., CD137). In some embodiments, the intracellular domain in the IL-15 polypeptide comprises a co-stimulatory signaling domain derived from OX40. In some embodiments, the intracellular domain in the IL-15 polypeptide comprises a co-stimulatory signaling domain derived from DAP10. In some embodiments, the intracellular domain in the IL-15 polypeptide comprises a co-stimulatory signaling domain derived from CD27.

Also within the scope of the present disclosure are variants of any of the co-stimulatory signaling domains described herein, such that the co-stimulatory signaling domain is capable of modulating the immune response of the immune cell. In some embodiments, the co-stimulatory signaling domains comprises up to 10 amino acid residue variations (e.g., 1, 2, 3, 4, 5, or 8) as compared to a wild-type counterpart. Such co-stimulatory signaling domains comprising one or more amino acid variations may be referred to as variants. Mutation of amino acid residues of the co-stimulatory signaling domain may result in an increase in signaling transduction and enhanced stimulation of immune responses relative to co-stimulatory signaling domains that do not comprise the mutation. Mutation of amino acid residues of the co-stimulatory signaling domain may result in a decrease in signaling transduction and reduced stimulation of immune responses relative to co-stimulatory signaling domains that do not comprise the mutation.

In some embodiments, the intracellular domain of the IL-15 polypeptide further comprises a primary intracellular signaling domain, such as an intracellular signaling domain of CD3ζ.

In some embodiments, the membrane-bound IL-15 polypeptide further comprises a signal peptide that targets the IL-15 polypeptide to the secretory pathway of the cell (e.g., ER) and will allow for integration and anchoring of the IL-15 polypeptide into the lipid bilayer of the host cell. Signal peptides including signal sequences of naturally occurring proteins or synthetic, non-naturally occurring signal sequences, which are compatible for use in the transmembrane IL-15 polypeptides described herein will be evident to one of skill in the art. In some embodiments, the signal peptide is derived from a molecule selected from the group consisting of CD8α, GM-CSF receptor α, IL-3, and IgG1 heavy chain. In some embodiments, the signal peptide is derived from CD8α.

In some embodiments, the IL-15 polypeptide comprises an IL-15 fragment and a GPI-anchoring peptide sequence, wherein the IL-15 fragment comprises an amino acid substitution at position 8, 62, 3 and/or 25, wherein numbering of the amino acid residue positions is according to SEQ ID NO: 1. In some embodiments, the IL-15 polypeptide comprises an amino acid substitution at position 62. In some embodiments, the amino acid substitution at position 62 is selected from the group consisting of T62G, T62I, T62Q, T62V, T62P, T62L, T62A, T62S and T62Y. In some embodiments, the amino acid substitution at position 62 is T62G. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 7. In some embodiments, the IL-15 polypeptide comprises SEQ ID NO: 7. In some embodiments, the IL-15 polypeptide comprises an amino acid substitution at position 8. In some embodiments, the amino acid substitution at position 8 is D8E. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 5. In some embodiments, the IL-15 polypeptide comprises SEQ ID NO: 5. In some embodiments, the IL-15 polypeptide comprises an amino acid substitution at position 3. In some embodiments, the amino acid substitution at position 3 is V3Y. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 78. In some embodiments, the IL-15 polypeptide comprises SEQ ID NO: 78. In some embodiments, the IL-15 polypeptide comprises an amino acid substitution at position 25. In some embodiments, the amino acid substitution at position 25 is selected from the group consisting of L25E and L25F. In some embodiments, the amino acid substitution at position 25 is L25F. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 79. In some embodiments, the IL-15 polypeptide comprises SEQ ID NO: 79. In some embodiments, the IL-15 polypeptide comprises amino acid substitutions at both position 8 and position 62. In some embodiments, the GPI-anchoring peptide sequence is attached to a GPI linker. In some embodiments, the GPI-anchoring peptide sequence is located at the C-terminus of the IL-15 polypeptide.

In some embodiments, the IL-15 polypeptide comprises an IL-15 fragment and a transmembrane domain, wherein the IL-15 fragment comprises an amino acid substitution at position 8, 62, 3 and/or 25, wherein numbering of the amino acid residue positions is according to SEQ ID NO: 1. In some embodiments, the IL-15 polypeptide comprises an amino acid substitution at position 62. In some embodiments, the amino acid substitution at position 62 is selected from the group consisting of T62G, T62I, T62Q, T62V, T62P, T62L, T62A, T62S and T62Y. In some embodiments, the amino acid substitution at position 62 is T62G. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 7. In some embodiments, the IL-15 polypeptide comprises SEQ ID NO: 7. In some embodiments, the IL-15 polypeptide comprises an amino acid substitution at position 8. In some embodiments, the amino acid substitution at position 8 is D8E. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 5. In some embodiments, the IL-15 polypeptide comprises SEQ ID NO: 5. In some embodiments, the IL-15 polypeptide comprises an amino acid substitution at position 3. In some embodiments, the amino acid substitution at position 3 is V3Y. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 78. In some embodiments, the IL-15 polypeptide comprises SEQ ID NO: 78. In some embodiments, the IL-15 polypeptide comprises an amino acid substitution at position 25. In some embodiments, the amino acid substitution at position 25 is selected from the group consisting of L25E and L25F. In some embodiments, the amino acid substitution at position 25 is L25F. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 79. In some embodiments, the IL-15 polypeptide comprises SEQ ID NO: 79. In some embodiments, the IL-15 polypeptide comprises amino acid substitutions at both position 8 and position 62. In some embodiments, the transmembrane domain is a transmembrane domain of IL-15Rα.

In some embodiments, the IL-15 polypeptide comprises an IL-15 fragment, a transmembrane domain and an intracellular domain, wherein the IL-15 fragment comprises an amino acid substitution at position 8, 62, 3 and/or 25 wherein numbering of the amino acid residue positions is according to SEQ ID NO: 1. In some embodiments, the IL-15 polypeptide comprises an amino acid substitution at position 62. In some embodiments, the amino acid substitution at position 62 is selected from the group consisting of T62G, T62I, T62Q, T62V, T62P, T62L, T62A, T62S and T62Y. In some embodiments, the amino acid substitution at position 62 is T62G. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 7. In some embodiments, the IL-15 polypeptide comprises SEQ ID NO: 7. In some embodiments, the IL-15 polypeptide comprises an amino acid substitution at position 8. In some embodiments, the amino acid substitution at position 8 is D8E. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 5. In some embodiments, the IL-15 polypeptide comprises SEQ ID NO: 5. In some embodiments, the IL-15 polypeptide comprises an amino acid substitution at position 3. In some embodiments, the amino acid substitution at position 3 is V3Y. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 78. In some embodiments, the IL-15 polypeptide comprises SEQ ID NO: 78. In some embodiments, the IL-15 polypeptide comprises an amino acid substitution at position 25. In some embodiments, the amino acid substitution at position 25 is selected from the group consisting of L25E and L25F. In some embodiments, the amino acid substitution at position 25 is L25F. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 79. In some embodiments, the IL-15 polypeptide comprises SEQ ID NO: 79. In some embodiments, the IL-15 polypeptide comprises amino acid substitutions at both position 8 and position 62. In some embodiments, the transmembrane domain is a CD4, CD3, CD8α, or CD28 transmembrane domain. In some embodiments, the IL-15 polypeptide further comprises a hinge domain, such as a hinge domain derived from CD8. In some embodiments, the intracellular domain comprises a primary intracellular signaling domain, such as an intracellular signaling domain of CD3ζ. In some embodiments, the intracellular domain comprises a co-stimulatory signaling domain. In some embodiments, the co-stimulatory signaling domain is derived from a co-stimulatory molecule selected from the group consisting of CD27, CD28, 4-1BB, OX40, DAP10, CD30, CD40, CD3, LFA-1, CD2, CD7, LIGHT, NKG2C, B7-H3, ligands of CD83 and combinations thereof. In some embodiments, the intracellular domain comprises a co-stimulatory signaling domain of 4-1BB and a primary intracellular signaling domain of CD3ζ.

In some embodiments, the IL-15 polypeptide comprises an IL-15 fragment, a transmembrane domain and a co-stimulatory signaling domain, wherein the IL-15 fragment comprises an amino acid substitution at position 8, 62, 3 and/or 25, wherein numbering of the amino acid residue positions is according to SEQ ID NO: 1. In some embodiments, the IL-15 polypeptide comprises an amino acid substitution at position 62. In some embodiments, the amino acid substitution at position 62 is selected from the group consisting of T62G, T62I, T62Q, T62V, T62P, T62L, T62A, T62S and T62Y. In some embodiments, the amino acid substitution at position 62 is T62G. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 7. In some embodiments, the IL-15 polypeptide comprises SEQ ID NO: 7. In some embodiments, the IL-15 polypeptide comprises an amino acid substitution at position 8. In some embodiments, the amino acid substitution at position 8 is D8E. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 5. In some embodiments, the IL-15 polypeptide comprises SEQ ID NO: 5. In some embodiments, the IL-15 polypeptide comprises an amino acid substitution at position 3. In some embodiments, the amino acid substitution at position 3 is V3Y. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 78. In some embodiments, the IL-15 polypeptide comprises SEQ ID NO: 78. In some embodiments, the IL-15 polypeptide comprises an amino acid substitution at position 25. In some embodiments, the amino acid substitution at position 25 is selected from the group consisting of L25E and L25F. In some embodiments, the amino acid substitution at position 25 is L25F. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 79. In some embodiments, the IL-15 polypeptide comprises SEQ ID NO: 79. In some embodiments, the IL-15 polypeptide comprises amino acid substitutions at both position 8 and position 62. In some embodiments, the transmembrane domain is a CD4, CD3, CD8α, or CD28 transmembrane domain. In some embodiments, the IL-15 polypeptide further comprises a hinge domain, such as a hinge domain derived from CD8. In some embodiments, the co-stimulatory signaling domain is derived from a co-stimulatory molecule selected from the group consisting of CD27, CD28, 4-1BB, OX40, DAP10, CD30, CD40, CD3, LFA-1, CD2, CD7, LIGHT, NKG2C, B7-H3, ligands of CD83 and combinations thereof.

The IL-15 polypeptide may comprise one or more peptide linkers disposed between different domains. For example, the IL-15 fragment and the second polypeptide fragment can be fused to each other via a peptide bond or via a peptide linker. The peptide linkers connecting different domains may be the same or different. Each peptide linker can be optimized individually. The peptide linker can be of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50 or more amino acids long. In some embodiments, the peptide linker is no more than about any of 50, 40, 35, 30, 25, 20, 19, 18, 17, 16, 15, 14, 13, 12, 11, 10, 9, 8, 7, 6, 5 or fewer amino acids long. In some embodiments, the length of the peptide linker is any of about 1 amino acid to about 10 amino acids, about 1 amino acids to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, about 30 amino acids to about 50 amino acids, or about 1 amino acid to about 50 amino acids.

The peptide linker may have a naturally occurring sequence, or a non-naturally occurring sequence. In some embodiments, the peptide linker is a flexible linker. Exemplary flexible linkers include glycine polymers (G)ₙ, glycine-serine polymers (including, for example, (GS)ₙ (SEQ ID NO: 67), (GSGGS)ₙ (SEQ ID NO: 68) and (GGGS)ₙ (SEQ ID NO: 69), where n is an integer of at least one), glycine-alanine polymers, alanine-serine polymers, and other flexible linkers known in the art. In some embodiments, the peptide linker has the amino acid sequence of SEQ ID NO: 40 or 41.

In some embodiments, the IL-15 polypeptide coding sequence lacks the sequences of some or all of the putative upstream start codons. In some embodiments, the IL-15 polypeptide may comprise certain amino acid mutations without effect on the binding of IL-15 with IL-15R (e.g., functional effect). In some embodiments, the IL-15 polypeptide comprises changed nucleotides (e.g., nucleotide substitutions, deletions, and/or additions). In some embodiments, the nucleotide changes occur in the mature IL-15 sequence to generate a mutant IL-15 polypeptide. The changed nucleotides may afford improved substrate specificity and function (e.g., anti-tumor effects) of the IL-15 polypeptide without overproduction of inflammatory cytokines.

Additional descriptions of IL-15 and IL-15 interactions are generally known in the art. See, for example, U.S. Pat. Nos. 9,389,236, 10,464,993, 9,629,877, EP1777294A1, U.S. Pat. Nos. 10,428,305, 7,998,736, 9,303,080, and 9,931,377, which are hereby incorporated by reference.

In some embodiments, the IL-15 polypeptide comprises an amino acid sequence variant of the IL-15 polypeptides described herein. For example, it may be desirable to modulate the binding affinity and/or other biological properties of the IL-15 polypeptide. Amino acid sequence variants of an IL-15 polypeptide thereof may be prepared by introducing appropriate modifications into the nucleotide sequence encoding the IL-15 polypeptide, or by peptide synthesis. Such modifications include, for example, deletions from, and/or insertions into and/or substitutions of residues within the amino acid sequences of the IL-15 polypeptide. Any combination of deletion, insertion, and substitution can be made to arrive at the final construct, provided that the final construct possesses the desired characteristics, e.g., TLR-binding and/or pro-inflammatory activities.

In some embodiments, the IL-15 polypeptide comprises one or more (e.g., at least 1, 2, 3, 4, 5, 10, 15, 20 amino acids or more) conservative substitutions compared to the sequence of any one of the IL-15 polypeptides described herein. In some embodiments, the IL-15 polypeptide comprises at least about 80% sequence identity, such as at least about any one of 85%, 87%, 90%, 91%, 92%, 93%, 94%, 95%, 96%, 97%, 98%, 99% or more sequence identity to the sequence of any one of the IL-15 polypeptides described herein. Like the IL-15 polypeptides described herein, the IL-15 polypeptide variants have similar anti-tumor activities and low toxicity.

Conservative substitutions are shown in Table A below.

TABLE A
CONSERVATIVE SUBSTITITIONS

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped into different classes according to common side-chain properties:
a. hydrophobic: Norleucine, Met, Ala, Val, Leu, Ile;
b. neutral hydrophilic: Cys, Ser, Thr, Asn, Gln;
c. acidic: Asp, Glu;
d. basic: His, Lys, Arg;
e. residues that influence chain orientation: Gly, Pro;
f. aromatic: Trp, Tyr, Phe.

Non-conservative substitutions will entail exchanging a member of one of these classes for another class.

One of skill in the art will recognize that any suitable method can be used for generating mutations in a gene of interest, including mutagenesis, polymerase chain reaction, homologous recombination, or any other genetic engineering technique known to a person of skill in the art. A mutation may involve a single nucleotide (such as a point mutation, which involves the removal, addition or substitution of a single nucleotide base within a DNA sequence) or it may involve the insertion or deletion of large numbers of nucleotides. Mutations can arise spontaneously as a result of events such as errors in the fidelity of DNA replication, or induced following exposure to chemical or physical mutagens. A mutation can also be site-directed through the use of particular targeting methods that are well known to persons of skill in the art.

A useful method for identification of residues or regions of a polypeptide that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the polypeptide agent with its target (e.g., IL-15 variant and IL-15 receptor) is affected. Further substitutions may be introduced at the amino acid locations demonstrating functional sensitivity to the initial substitutions. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues.

In some embodiments, a peptide tag (typically a short peptide sequence able to be recognized by available antisera or compounds) may be included for following expression and trafficking of the IL-15 polypeptide. A vast variety of tag peptides can be used in the IL-15 polypeptide described herein, without limitation, PK tag, FLAG octapeptide, MYC tag, HIS tag (usually a stretch of 4 to 10 histidine residues) and e-tag (U.S. Pat. No. 6,686,152). The tag peptide(s) may be independently positioned at the N-terminus of the protein, at its C-terminus, internally, or at any of these positions when several tags are employed. Tag peptides can be detected by immunodetection assays using anti-tag antibodies.

### Engineered Receptor

Any of the modified immune cells described above may further express an engineered receptor. Exemplary engineered receptor include, but are not limited to, CAR, engineered TCR, and TAC receptors. In some embodiments, the engineered receptor comprises an extracellular domain that specifically binds to an antigen (e.g., a tumor antigen), a transmembrane domain, and an intracellular signaling domain. In some embodiments, the intracellular signaling domain comprises a primary intracellular signaling domain and/or a co-stimulatory domain. In some embodiments, the intracellular signaling domain comprises an intracellular signaling domain of a TCR co-receptor. In some embodiments, the engineered receptor is encoded by the heterologous nucleic acid sequence encoding the IL-15 polypeptide. In some embodiments, the engineered receptor is encoded by a second heterologous nucleic acid operably linked to a promoter (such as a constitutive promoter or an inducible promoter). In some embodiments, the engineered receptor is introduced to the modified immune cell by inserting proteins into the cell membrane while passing cells through a microfluidic system, such as CELL SQUEEZE® (see, for example, U.S. Patent Application Publication No. 20140287509). The engineered receptor may enhance the function of the modified immune cell, such as by targeting the modified immune cell, by transducing signals, and/or by enhancing cytotoxicity of the modified immune cell. In some embodiments, the modified immune cell does not express an engineered receptor, such as CAR, TCR, or TAC receptor.

In some embodiments, the engineered receptor comprises one or more specific binding domains that target at least one tumor antigen, and one or more intracellular effector domains, such as one or more primary intracellular signaling domains and/or co-stimulatory domains.

In some embodiments, the engineered receptor is a chimeric antigen receptor (CAR). Many chimeric antigen receptors are known in the art and may be suitable for the modified immune cell of the present application. CARs can also be constructed with a specificity for any cell surface marker by utilizing antigen binding fragments or antibody variable domains of, for example, antibody molecules. Any method for producing a CAR may be used herein. See, for example, U.S. Pat. Nos. 6,410,319, 7,446,191, 7,514,537, 9,765,342B2, WO 2002/077029, WO2015/142675, US2010/065818, US 2010/025177, US 2007/059298, WO2017025038A1, and Berger C. et al., J. Clinical Investigation 118: 1 294-308 (2008), which are hereby incorporated by reference. In some embodiments, the modified immune cell is a CAR-T cell.

CARs of the present application comprise an extracellular domain comprising at least one targeting domain that specifically binds at least one tumor antigen, a transmembrane domain, and an intracellular signaling domain. In some embodiments, the intracellular signaling domain generates a signal that promotes an immune effector function of the CAR-containing cell, e.g., a CAR-T cell. “Immune effector function or immune effector response” refers to function or response, e.g., of an immune effector cell, that enhances or promotes an immune attack of a target cell. For example, an immune effector function or response may refer to a property of a T or NK cell that promotes killing or the inhibition of growth or proliferation, of a target cell. Examples of immune effector function, e.g., in a CAR-T cell, include cytolytic activity (such as antibody-dependent cellular toxicity, or ADCC) and helper activity (such as the secretion of cytokines). In some embodiments, the CAR has an intracellular signaling domain with an attenuated immune effector function. In some embodiments, the CAR has an intracellular signaling domain having no more than about any of 90%, 80%, 70%, 60%, 50%, 40%, 30%, 20%, 10% or less of an immune effector function (such as cytolytic function against target cells) compared to a CAR having a full-length and wildtype CD3ζ and optionally one or more co-stimulatory domains. In some embodiments, the intracellular signaling domain generates a signal that promotes proliferation and/or survival of the CAR containing cell. In some embodiments, the CAR comprises one or more intracellular signaling domains selected from the signaling domains of CD28, CD137, CD3, CD27, CD40, ICOS, GITR, and OX40. The signaling domain of a naturally occurring molecule can comprise the entire intracellular (i.e., cytoplasmic) portion, or the entire native intracellular signaling domain, of the molecule, or a fragment or derivative thereof.

In some embodiments, the intracellular signaling domain of a CAR comprises a primary intracellular signaling domain. “Primary intracellular signaling domain” refers to cytoplasmic signaling sequence that acts in a stimulatory manner to induce immune effector functions. In some embodiments, the primary intracellular signaling domain contains a signaling motif known as Immunoreceptor Tyrosine-based Activation Motif, or ITAM. In some embodiments, the primary intracellular signaling domain comprises a functional signaling domain of a protein selected from the group consisting of CD3 zeta, CD3 gamma, CD3 delta, CD3 epsilon, common FcR gamma (FCER1G), FcR beta (Fc Epsilon Rib), CD79a, CD79b, Fcgamma RIIa, DAP10, and DAP12. In some embodiments, the primary intracellular signaling domain comprises a nonfunctional or attenuated signaling domain of a protein selected from the group consisting of CD3 zeta, CD3 gamma, CD3 delta, CD3 epsilon, common FcR gamma (FCER1G), FcR beta (Fc Epsilon Rib), CD79a, CD79b, Fcgamma RIIa, DAP10, and DAP12. The nonfunctional or attenuated signaling domain can be a mutant signaling domain having a point mutation, insertion or deletion that attenuates or abolishes one or more immune effector functions, such as cytolytic activity or helper activity, including antibody-dependent cellular toxicity (ADCC). In some embodiments, the CAR comprises a nonfunctional or attenuated CD3 zeta (i.e., CD3ζ or CD3z) signaling domain. In some embodiments, the intracellular signaling domain does not comprise a primary intracellular signaling domain. An attenuated primary intracellular signaling domain may induce no more than about any of 90%, 80%, 70%, 60%, 50%, 40%, 30%, 20%, 10% or less of an immune effector function (such as cytolytic function against target cells) compared to CARs having the same construct, but with the wildtype primary intracellular signaling domain.

In some embodiments, the intracellular signaling domain of a CAR comprises one or more (such as any of 1, 2, 3, or more) co-stimulatory domains. “Co-stimulatory domain” can be the intracellular portion of a co-stimulatory molecule. The term “co-stimulatory molecule” refers to a cognate binding partner on an immune cell (such as T cell) that specifically binds with a co-stimulatory ligand, thereby mediating a co-stimulatory response by the immune cell, such as, but not limited to, proliferation and survival. Co-stimulatory molecules are cell surface molecules other than antigen receptors or their ligands that contribute to an efficient immune response. A co-stimulatory molecule can be represented in the following protein families: TNF receptor proteins, Immunoglobulin-like proteins, cytokine receptors, integrins, signaling lymphocytic activation molecules (SLAM proteins), and activating NK cell receptors. Co-stimulatory molecules include, but are not limited to an MHC class I molecule, BTLA and a Toll ligand receptor, as well as OX40, CD27, CD28, CDS, ICAM-1, LFA-1 (CD11a/CD18), ICOS (CD278), and 4-1BB (CD137). Further examples of such co-stimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11 d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CDIOO (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

In some embodiments, the CAR comprises a single co-stimulatory domain. In some embodiments, the CAR comprises two or more co-stimulatory domains. In some embodiments, the intracellular signaling domain comprises a functional primary intracellular signaling domain and one or more co-stimulatory domains. In some embodiments, the CAR does not comprise a functional primary intracellular signaling domain (such as CD3ζ). In some embodiments, the CAR comprises an intracellular signaling domain consisting of or consisting essentially of one or more co-stimulatory domains. In some embodiments, the CAR comprises an intracellular signaling domain consisting of or consisting essentially of a nonfunctional or attenuated primary intracellular signaling domain (such as a mutant CD3ζ) and one or more co-stimulatory domains. Upon binding of the targeting domain to tumor antigen, the co-stimulatory domains of the CAR may transduce signals for enhanced proliferation, survival and differentiation of the engineered immune cells having the CAR (such as T cells), and inhibit activation induced cell death. In some embodiments, the one or more co-stimulatory signaling domains are derived from one or more molecules selected from the group consisting of CD27, CD28, 4-1BB (i.e., CD137), OX40, CD30, CD40, CD3, lymphocyte function-associated antigen-1(LFA-1), CD2, CD7, LIGHT, NKG2C, B7-H3 and ligands that specially bind to CD83.

In some embodiments, the intracellular signaling domain of the CAR comprises a co-stimulatory signaling domain derived from CD28. In some embodiments, the intracellular signaling domain comprises a cytoplasmic signaling domain of CD3ζ and a co-stimulatory signaling domain of CD28. In some embodiments, the intracellular signaling domain in the chimeric receptor of the present application comprises a co-stimulatory signaling domain derived from 4-1BB (i.e., CD137). In some embodiments, the intracellular signaling domain comprises a cytoplasmic signaling domain of CD3ζ and a co-stimulatory signaling domain of 4-1BB.

In some embodiments, the intracellular signaling domain of the CAR comprises a co-stimulatory signaling domain of CD28 and a co-stimulatory signaling domain of 4-1BB. In some embodiments, the intracellular signaling domain comprises a cytoplasmic signaling domain of CD3ζ, a co-stimulatory signaling domain of CD28, and a co-stimulatory signaling domain of 4-1BB. In some embodiments, the intracellular signaling domain comprises a polypeptide comprising from the N-terminus to the C-terminus: a co-stimulatory signaling domain of CD28, a co-stimulatory signaling domain of 4-1BB, and a cytoplasmic signaling domain of CD3ζ.

In some embodiments, the targeting domain of the CAR is an antibody or an antibody fragment, such as an scFv, a Fv, a Fab, a (Fab')₂, a single domain antibody (sdAb), or a VHH domain. In some embodiments, the targeting domain of the CAR is a ligand or an extracellular portion of a receptor that specifically binds to a tumor antigen. In some embodiments, the one or more targeting domains of the CAR specifically bind to a single tumor antigen. In some embodiments, the CAR is a bispecific or multispecific CAR with targeting domains that bind two or more tumor antigens. In some embodiments, the tumor antigen is selected from the group consisting of GPC3, CD19, BCMA, NY-ESO-1, VEGFR2, MAGE-A3, CD20, CD22, CD33, CD3ε, CEA, EGFR (such as EGFRvIII), GD2, HER2, IGF1R, mesothelin, PSMA, ROR1, WT1, and other tumor antigens with clinical significance, and combinations thereof. In some embodiments, the CAR specifically binds to a target antigen selected from the group consisting of BCMA, NY-ESO-1, VEGFR2, MAGE-A3, AFP, CD4, CD19, CD20, CD22, CD30, CD33, CD3ε, CD70, CD123, CEA, EGFR (such as EGFRvIII), GD2, GPC-2, GPC3, CLDN18.2, HER2, LILRB4, IL-13Rα2, IGF1R, mesothelin, PSMA, ROR1, WT1, NKG2D, CLL1, TGFaRII, TGFbRII, CCR5, CXCR4, CCR4, HPV related antigens, and EBV related antigens (e.g., LMP1 or LMP2).

In some embodiments, the CAR is a BCMA CAR. B cell mature antigen (BCMA), also known as CD269, is a member of the tumor necrosis factor receptor superfamily (Thompson et al., J. Exp. Medicine, 192 (1): 129-135, 2000). Human BCMA is expressed in plasma cells, and can bind B-cell activating factor (BAFF) and a proliferation including ligand (April) (e.g. Mackay et al., 2003 and Kalled et al., Immunological Review, 204: 43-54, 2005). BCMA may be used as a target antigen for immunotherapeutic agents, such as CAR-T cells, against various cancers. Thus, anti-BCMA antibodies (e.g., BCMA single domain antibodies) can be used in combination with cell immunotherapy using CAR-T cells to enhance cytotoxic effects against tumor cells. A wide variety of antigen binding domain sequences can be used as the targeting domains of the BCMA CAR. See, e.g., WO2017/025038, which is incorporated herein in its entirety. In some embodiments, the BCMA CAR comprises from the N-terminus to the C-terminus: a CD8 signal peptide (SP), an anti-BCMA sdAb, a CD8 hinge, a CD8 transmembrane, a 4-1BB intracellular co-stimulatory domain, and a CD3ζ intracellular signaling domain. In some embodiments, the BCMA CAR comprises from the N-terminus to the C-terminus: a CD8α signal peptide, a first anti-BCMA VHH, an optional linker, a second anti-BCMA VHH, a CD8α hinge, a CD8α transmembrane, a 4-1BB intracellular co-stimulatory domain, and a CD3ζ intracellular signaling domain. In some embodiments, the anti-BCMA VHH—VHH domains comprise the amino acid sequence of SEQ ID NO: 23. In some embodiments, the BCMA CAR comprises the amino acid sequence of SEQ ID NO: 26.

In some embodiments, the CAR is a CD19 CAR. CD19 is a B-cell surface protein expressed throughout B-cell development; therefore, it is expressed on nearly all B-cell malignancies, including several types of leukemia and many non-Hodgkin lymphomas (Scheuermann RH and Racila E. Leuk Lymphoma. 1995; 18(5-6):385-397). The near-universal expression and specificity for a single cell lineage has made CD19 an attractive target for CAR-modified T-cell therapies. A wide variety of antigen binding domain sequences can be used as the targeting domains of the CD19 CAR. See, e.g., WO2012/079000, which is incorporated herein in its entirety. In some embodiments, the CD19 CAR comprises from the N-terminus to the C-terminus: a CD8α signal peptide, a CD19 scFv, a CD8α hinge, a CD8α transmembrane, a 4-1BB intracellular co-stimulatory domain, and a CD3ζ intracellular signaling domain. In some embodiments, the anti-CD19 scFv comprises the amino acid sequence of SEQ ID NO: 24. In some embodiments, the CD19 CAR comprises the amino acid sequence of SEQ ID NO: 27.

In some embodiments, the CAR is a GPC3 CAR. Glypican-3 (GPC3) is a member of the glypican family, a group of heparan sulfate proteoglycans linked to the cell surface through a glycosyl—phosphatidylinositol anchor. GPC3 is highly expressed on a variety of pediatric solid embryonal tumors including the majority of hepatoblastomas, Wilms tumors, rhabdoid tumors, certain germ cell tumor subtypes, and a minority of rhabdomyosarcomas. A wide variety of antigen binding domain sequences can be used as the targeting domains of the GPC3 CAR. See, e.g., WO2016/049459, which is incorporated herein in its entirety. In some embodiments, the GPC3 CAR comprises from the N-terminus to the C-terminus: a CD8α signal peptide, a GPC3 scFv, a CD8α hinge, a CD8α transmembrane, a 4-1BB intracellular co-stimulatory domain, and a CD3ζ intracellular signaling domain. In some embodiments, the anti-GPC3 scFv comprises the amino acid sequence of SEQ ID NO: 80. In some embodiments, the GPC3 CAR comprises the amino acid sequence of SEQ ID NO: 81.

In some embodiments, the transmembrane domain of the CAR comprises a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL-2R beta, IL-2R gamma, IL-7R a, ITGA1, VLA1, CD49a, ITGA4, IA4, CD49D, ITGA6, VLA-6, CD49f, ITGAD, CD11 d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Lyl08), SLAM (SLAMF1, CD150, IPO-3), BLAME (SLAMF8), SELPLG (CD162), LTBR, PAG/Cbp, NKp44, NKp30, NKp46, NKG2D, and/or NKG2C. In some embodiments, the transmembrane domain of the CAR is a CD4, CD3, CD8α, or CD28 transmembrane domain. In some embodiments, the transmembrane domain of the CAR comprises a transmembrane domain of CD8α.

In some embodiments, the extracellular domain is connected to the transmembrane domain by a hinge region. In one embodiment, the hinge region comprises the hinge region of CD8α.

In some embodiments, the CAR comprises a signal peptide, such as a CD8αSP.

In some embodiments, the engineered receptor is a modified T-cell receptor. In some embodiments, the engineered TCR is specific for a tumor antigen. In some embodiments, the tumor antigen is selected from the group consisting of GPC3, CD19, BCMA, NY-ESO-1, VEGFR2, MAGE-A3, VEGFR2, MAGE-A3, CD20, CD22, CD33, CD3ε, CEA, EGFR (such as EGFRvIII), GD2, HER2, IGF1R, mesothelin, PSMA, ROR1, WT1, and other tumor antigens with clinical significance. In some embodiments, the tumor antigen is derived from an intracellular protein of tumor cells. Many TCRs specific for tumor antigens (including tumor-associated antigens) have been described, including, for example, NY-ESO-1 cancer-testis antigen, the p53 tumor suppressor antigens, TCRs for tumor antigens in melanoma (e.g., MARTI, gp 100), leukemia (e.g., WT1, minor histocompatibility antigens), and breast cancer (HER2, NY-BR1, for example). Any of the TCRs known in the art may be used in the present application. In some embodiments, the TCR has an enhanced affinity to the tumor antigen. Exemplary TCRs and methods for introducing the TCRs to immune cells have been described, for example, in U.S. Pat. No. 5,830,755, and Kessels et al. Immunotherapy through TCR gene transfer. Nat. Immunol. 2, 957-961 (2001). In some embodiments, the modified immune cell is a TCR-T cell.

The TCR receptor complex is an octameric complex formed by variable TCR receptor α and β chains (γ and ζ chains on case of γδ T cells) with three dimeric signaling modules CD3δ/ε, CD3γ/ε and CD247 (T-cell surface glycoprotein CD3 zeta chain) ζ/ζ or ζ/η. Ionizable residues in the transmembrane domain of each subunit form a polar network of interactions that hold the complex together. TCR complex has the function of activating signaling cascades in T cells.

In some embodiments, the engineered receptor is an engineered TCR comprising one or more T-cell receptor (TCR) fusion proteins (TFPs). Exemplary TFPs have been described, for example, in US20170166622A1, which is incorporated herein by reference. In some embodiments, the TFP comprises an extracellular domain of a TCR subunit that comprises an extracellular domain or portion thereof of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications. In some embodiments, the TFP comprises a transmembrane domain that comprises a transmembrane domain of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications. In some embodiments, the TFP comprises a transmembrane domain that comprises a transmembrane domain of a protein selected from the group consisting of a TCR alpha chain, a TCR beta chain, a TCR zeta chain, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications.

In some embodiments, the TFP comprising a TCR subunit comprising at least a portion of a TCR extracellular domain, and a TCR intracellular domain comprising a stimulatory domain from an intracellular signaling domain of CD3 epsilon; and an antigen binding domain, wherein the TCR subunit and the antigen binding domain are operatively linked, and wherein the TFP incorporates into a TCR when expressed in a T cell.

In some embodiments, the TFP comprises a TCR subunit comprising at least a portion of a TCR extracellular domain, and a TCR intracellular domain comprising a stimulatory domain from an intracellular signaling domain of CD3 gamma; and an antigen binding domain wherein the TCR subunit and the antigen binding domain are operatively linked, and wherein the TFP incorporates into a TCR when expressed in a T cell.

In some embodiments, the TFP comprises a TCR subunit comprising at least a portion of a TCR extracellular domain, and a TCR intracellular domain comprising a stimulatory domain from an intracellular signaling domain of CD3 delta; and an antigen binding domain, wherein the TCR subunit and the antigen binding domain are operatively linked, and wherein the TFP incorporates into a TCR when expressed in a T cell.

In some embodiments, the TFP comprises a TCR subunit comprising at least a portion of a TCR extracellular domain, and a TCR intracellular domain comprising a stimulatory domain from an intracellular signaling domain of TCR alpha; and an antigen binding domain wherein the TCR subunit and the antigen binding domain are operatively linked, and wherein the TFP incorporates into a TCR when expressed in a T cell.

In some embodiments, the TFP comprises a TCR subunit comprising at least a portion of a TCR extracellular domain, and a TCR intracellular domain comprising a stimulatory domain from an intracellular signaling domain of TCR beta; and an antigen binding domain wherein the TCR subunit and the antigen binding domain are operatively linked, and wherein the TFP incorporates into a TCR when expressed in a T cell.

In some embodiments, the engineered receptor is a T-cell antigen coupler (TAC) receptor. Exemplary TAC receptors have been described, for example, in US20160368964A1, which is incorporated herein by reference. In some embodiments, the TAC comprises a targeting domain, a TCR-binding domain that specifically binds a protein associated with the TCR complex, and a T-cell receptor signaling domain. In some embodiments, the targeting domain is an antibody fragment, such as scFv or V_{H}H, which specifically binds to a tumor antigen. In some embodiments, the targeting domain is a designed Ankyrin repeat (DARPin) polypeptide. In some embodiments, the tumor antigen is selected from the group consisting of GPC3, CD19, BCMA, NY-ESO-1, VEGFR2, MAGE-A3, VEGFR2, MAGE-A3, CD20, CD22, CD33, CD3ε, CEA, EGFR (such as EGFRvIII), GD2, HER2, IGF1R, mesothelin, PSMA, ROR1, WT1, and other tumor antigens with clinical significance. In some embodiments, the protein associated with the TCR complex is CD3, such as CD3ε. In some embodiments, the TCR-binding domain is a single chain antibody, such as scFv, or a V_{H}H. In some embodiments, the TCR-binding domain is derived from UCHT1. In some embodiments, the TAC receptor comprises a cytosolic domain and a transmembrane domain. In some embodiments, the T-cell receptor signaling domain comprises a cytosolic domain derived from a TCR co-receptor. Exemplary TCR co-receptors include, but are not limited to, CD4, CD8, CD28, CD45, CD4, CD5, CD9, CD16, CD22, CD33, CD37, CD64, CD80, CD86, CD134, CD137 and CD154. In some embodiments, the TAC receptor comprises a transmembrane domain and a cytosolic domain derived from CD4. In some embodiments, the TAC receptor comprises a transmembrane domain and a cytosolic domain derived from CD8 (such as CD8α).

T cell co-receptors are expressed as membrane protein on T cells. They can provide stabilization of the TCR: peptide: MHC complex and facilitate signal transduction. The two subtypes of T cell co-receptor, CD4 and CD8, display strong specificity for particular MHC classes. The CD4 co-receptor can only stabilize TCR: MHC II complexes while the CD8 co-receptor can only stabilize the TCR: MHC I complex. The differential expression of CD4 and CD8 on different T cell types results in distinct T cell functional subpopulations. CD8+ T cells are cytotoxic T cells.

CD4 is a glycoprotein expressed on the surface of immune cells such as T helper cells, monocytes, macrophages, and dendritic cells. CD4 has four immunoglobulin domains (Di to D4) exposed on the extracellular cell surface. CD4 contains a special sequence of amino acids on its short cytoplasmic/intracellular tail, which allow CD4 tail to recruit and interact with the tyrosine kinase Lck. When the TCR complex and CD4 each bind to distinct regions of the MHC II molecule, the close proximity between the TCR complex and CD4 allows Lck bound to the cytoplasmic tail of CD4 to tyrosine-phosphorylate the Immunoreceptor Tyrosine Activation Motifs (ITAM) on the cytoplasmic domains of CD3, thus amplifying TCR generated signal.

CD8 is a glycoprotein of either a homodimer composed of two a chains (less common), or a heterodimer composed of one α and one β chain (more common), each comprising an immunoglobulin variable (IgV)—like extracellular domain connected to the membrane by a thin stalk, and an intracellular tail. CD8 is predominantly expressed on the surface of cytotoxic T cells, but can also be found on natural killer cells, cortical thymocytes, and dendritic cells. The CD8 cytoplasmic tail interacts with Lck, which phosphorylates the cytoplasmic CD3 and ζ-chains of the TCR complex once TCR binds its specific antigen. Tyrosine-phosphorylation on the cytoplasmic CD3 and ζ-chains initiates a cascade of phosphorylation, eventually leading to gene transcription.

In some embodiments, the modified immune cell expresses more than one engineered receptors, such as any combination of CAR, TCR, TAC receptor.

In some embodiments, the engineered receptor (such as CAR, TCR, or TAC) expressed by the modified immune cell targets one or more tumor antigens. Tumor antigens are proteins that are produced by tumor cells that can elicit an immune response, particularly T-cell mediated immune responses. The selection of the targeted antigen of the disclosure will depend on the particular type of cancer to be treated. Exemplary tumor antigens include, for example, a glioma-associated antigen, carcinoembryonic antigen (CEA), β-human chorionic gonadotropin, alphafetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, HER2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In some embodiments, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and gp100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B cell differentiation antigens such as CD19, CD20 and CD37 are other candidates for target antigens in B-cell lymphoma.

In some embodiments, the tumor antigen is a tumor-specific antigen (TSA) or a tumor-associated antigen (TAA). A TSA is unique to tumor cells and does not occur on other cells in the body. A TAA associated antigen is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells.

Non-limiting examples of TSA or TAA antigens include the following: Differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alphafetoprotein, beta-HCG, BCA225, BTAA, CA 125, CA 15-3/CA 27.29/BCAA, CA 195, CA 242, CA-50, CAM43, CD68P1, CO-029, FGF-5, G250, Ga733/EpCAM, HTgp-175, M344, MA-50, MG7-Ag, MOV18, NB/70K, NY-CO-1, RCAS 1, SDCCAGi6, TA-90Mac-2 binding protein/cyclophilin C-associated protein, TAAL6, TAG72, TLP, and TPS.

### Nucleic Acids

The modified immune cells described herein comprises one or more heterologous nucleic acids sequence(s) encoding any one of the IL-15 polypeptides and/or engineered receptors described herein.

Another aspect of the present application provides an isolated nucleic acid comprising a nucleic acid sequence encoding any one of the IL-15 polypeptides described herein. In some embodiments, there is provided an isolated nucleic acid comprising a nucleic acid sequence encoding any one of the engineered receptors described herein. In some embodiments, the nucleic acid is a DNA. In some embodiments, the nucleic acid is a RNA. In some embodiments, the nucleic acid is linear. In some embodiments, the nucleic acid is circular.

The nucleic acid sequence encoding the IL-15 polypeptide and/or the nucleic acid encoding the engineered receptor may be operably linked to one or more regulatory sequences. Exemplary regulatory sequences that control the transcription and/or translation of a coding sequence are known in the art and may include, but not limited to, a promoter, additional elements for proper initiation, regulation and/or termination of transcription (e.g. polyA transcription termination sequences), mRNA transport (e.g. nuclear localization signal sequences), processing (e.g. splicing signals), stability (e.g. introns and non-coding 5′ and 3′ sequences), translation (e.g. an initiator Met, tripartite leader sequences, IRES ribosome binding sites, signal peptides, etc.), and insertion site for introducing an insert into the viral vector. In some embodiments, the regulatory sequence is a promoter, a transcriptional enhancer and/or a sequence that allows for proper expression of the IL-15 polypeptide and/or the engineered receptor.

The term “regulatory sequence” or “control sequence” refers to a DNA sequence that affects the expression of a coding sequence to which it is operably linked. The nature of such regulatory sequences differs depending upon the host organism. In prokaryotes, regulatory sequences generally include promoters, ribosomal binding sites, and terminators. In eukaryotes, regulatory sequences include promoters, terminators and, in some instances, enhancers, transactivators or transcription factors.

The term “operably linked” refers to a juxtaposition wherein the components so described are in a relationship permitting them to function in their intended manner. A regulatory sequence “operably linked” to a coding sequence is ligated in such a way that expression of the coding sequence is achieved under conditions compatible with the regulatory sequences.

As used herein, a “promoter” or a “promoter region” refers to a segment of DNA or RNA that controls transcription of the DNA or RNA to which it is operatively linked.

The promoter region includes specific sequences that are involved in RNA polymerase recognition, binding and transcription initiation. In addition, the promoter includes sequences that modulate recognition, binding and transcription initiation activity of RNA polymerase (i.e., binding of one or more transcription factors). These sequences can be cis acting or can be responsive to trans acting factors. Promoters, depending upon the nature of the regulation, can be constitutive or regulated. Regulated promoters can be inducible or environmentally responsive (e.g. respond to cues such as pH, anaerobic conditions, osmoticum, temperature, light, or cell density). Many
such promoter sequences are known in the art. See, for example, U.S. Pat. Nos. 4,980,285; 5,631,150; 5,707,928; 5,759,828; 5,888,783; 5,919,670, and, Sambrook, et al., Molecular Cloning: A Laboratory Manual, 2nd Ed., Cold Spring Harbor Press (1989).

In some embodiments, the nucleic acid sequence encoding the IL-15 polypeptide is operably linked to a first promoter. In some embodiments, the nucleic acid sequence encoding the engineered receptor is operably linked to a second promoter. In some embodiments, the nucleic acid sequence encoding the IL-15 polypeptide and the nucleic acid sequence encoding the engineered receptor are operably linked to the same promoter. In some embodiments, the nucleic acid sequence encoding the IL-15 polypeptide and the nucleic acid sequence encoding the engineered receptor are operably linked to separate promoters.

In some embodiments, the promoter is an endogenous promoter. For example, a nucleic acid encoding the IL-15 polypeptide and/or the engineered receptor may be knocked-in to the genome of the modified immune cell downstream of an endogenous promoter using any methods known in the art, such as CRISPR/Cas9 method. In some embodiments, the endogenous promoter is a promoter for an abundant protein, such as beta-actin. In some embodiments, the endogenous promoter is an inducible promoter, for example, inducible by an endogenous activation signal of the modified immune cell. In some embodiments, wherein the modified immune cell is a T cell, the promoter is a T cell activation-dependent promoter (such as an IL-2 promoter, an NFAT promoter, or an NF_{K}B promoter). In some embodiments, the promoter is a heterologous promoter.

Varieties of promoters have been explored for gene expression in mammalian cells, and any of the promoters known in the art may be used in the present application. Promoters may be roughly categorized as constitutive promoters or regulated promoters, such as inducible promoters. In some embodiments, the heterologous nucleic acid sequence encoding the IL-15 polypeptide and/or the engineered receptor is operably linked to a constitutive promoter. In some embodiments, the heterologous nucleic acid sequence encoding the IL-15 polypeptide and/or the engineered receptor is operably linked to an inducible promoter. In some embodiments, a constitutive promoter is operably linked to the nucleic acid sequence encoding the IL-15 polypeptide, and an inducible promoter is operably linked to the nucleic acid sequence encoding the engineered receptor. In some embodiments, a constitutive promoter is operably linked to the nucleic acid sequence encoding the engineered receptor, and an inducible promoter is operably linked to the nucleic acid sequence encoding the IL-15 polypeptide. In some embodiments, a first inducible promoter is operably linked to the nucleic acid sequence encoding the IL-15 polypeptide, and a second inducible promoter is operably linked to the nucleic acid sequence encoding the engineered receptor. In some embodiments, the first inducible promoter is inducible by a first inducing condition, and the second inducible promoter is inducible by a second inducing condition. In some embodiments, the first inducing condition is the same as the second inducing condition. In some embodiments, the first inducible promoter and the second inducible promoter are induced simultaneously. In some embodiments, the first inducible promoter and the second inducible promoter are induced sequentially, for example, the first inducible promoter is induced prior to the second inducible promoter, or the first inducible promoter is induced after the second inducible promoter.

Constitutive promoters allow heterologous genes (also referred to as transgenes) to be expressed constitutively in the host cells. Exemplary constitutive promoters contemplated herein include, but are not limited to, Cytomegalovirus (CMV) promoters, human elongation factors-1alpha (hEF1α), ubiquitin C promoter (UbiC), phosphoglycerokinase promoter (PGK), simian virus 40 early promoter (SV40), and chicken β-Actin promoter coupled with CMV early enhancer (CAGG). The efficiencies of such constitutive promoters on driving transgene expression have been widely compared in a huge number of studies. In some embodiments, the promoter is a hEF1α promoter.

In some embodiments, the promoter is an inducible promoter. Inducible promoters belong to the category of regulated promoters. The inducible promoter can be induced by one or more conditions, such as a physical condition, microenvironment of the modified immune cell, or the physiological state of the modified immune cell, an inducer (i.e., an inducing agent), or a combination thereof. In some embodiments, the inducing condition does not induce the expression of endogenous genes in the modified immune cell, and/or in the subject that receives the pharmaceutical composition. In some embodiments, the inducing condition is selected from the group consisting of: inducer, irradiation (such as ionizing radiation, light), temperature (such as heat), redox state, tumor environment, and the activation state of the modified immune cell.

In some embodiments, the promoter is inducible by an inducer. In some embodiments, the inducer is a small molecule, such as a chemical compound. In some embodiments, the small molecule is selected from the group consisting of doxycycline, tetracycline, alcohol, metal, or steroids. Chemically-induced promoters have been most widely explored. Such promoters includes promoters whose transcriptional activity is regulated by the presence or absence of a small molecule chemical, such as doxycycline, tetracycline, alcohol, steroids, metal and other compounds. Doxycycline-inducible system with reverse tetracycline-controlled transactivator (rtTA) and tetracycline-responsive element promoter (TRE) is the most established system at present. WO9429442 describes the tight control of gene expression in eukaryotic cells by tetracycline responsive promoters. WO9601313 discloses tetracycline-regulated transcriptional modulators. Additionally, Tet technology, such as the Tet-on system, has described, for example, on the website of TetSystems.com. Any of the known chemically regulated promoters may be used to drive expression of the therapeutic protein in the present application.

In some embodiments, the inducer is a polypeptide, such as a growth factor, a hormone, or a ligand to a cell surface receptor, for example, a polypeptide that specifically binds a tumor antigen. In some embodiments, the polypeptide is expressed by the modified immune cell. In some embodiments, the polypeptide is encoded by a nucleic acid in the heterologous nucleic acid. Many polypeptide inducers are also known in the art, and they may be suitable for use in the present application. For example, ecdysone receptor-based gene switches, progesterone receptor-based gene switches, and estrogen receptor based gene switches belong to gene switches employing steroid receptor derived transactivators (WO9637609 and WO9738117 etc.).

In some embodiments, the inducer comprises both a small molecule component and one or more polypeptides. For example, inducible promoters that dependent on dimerization of polypeptides are known in the art, and may be suitable for use in the present application. The first small molecule CID system, developed in 1993, used FK1012, a derivative of the drug FK506, to induce homo-dimerization of FKBP. By employing similar strategies, Wu et al successfully make the CAR-T cells titratable through an ON-switch manner by using Rapalog/FKPB-FRB* and Gibberelline/GID1-GAI dimerization dependent gene switch (C.-Y. Wu et al., Science 350, aab4077 (2015)). Other dimerization dependent switch systems include Coumermycin/GyrB-GyrB (Nature 383 (6596): 178-81), and HaXS/Snap-tag-HaloTag (Chemistry and Biology 20 (4): 549-57).

In some embodiments, the promoter is a light-inducible promoter, and the inducing condition is light. Light inducible promoters for regulating gene expression in mammalian cells are also well-known in the art (see, for example, Science 332, 1565-1568 (2011); Nat. Methods 9, 266-269 (2012); Nature 500: 472-476 (2013); Nature Neuroscience 18:1202-1212 (2015)). Such gene regulation systems can be roughly divided into two categories based on their regulations of (1) DNA binding or (2) recruitment of a transcriptional activation domain to a DNA bound protein. For instance, synthetic mammalian blue light controlled transcription system based on melanopsin which, in response to blue light (480 nm), triggers an intracellular calcium increase that result in calcineurin-mediated mobilization of NFAT, were developed and tested in mammalian cells. More recently, Motta-Mena et al described a new inducible gene expression system developed from naturally occurring EL222 transcription factor that confers high-level, blue light-sensitive control of transcriptional initiation in human cell lines and zebrafish embryos (Nat. Chem. Biol. 10(3):196-202 (2014)). Additionally, the red light induced interaction of photoreceptor phytochrome B (PhyB) and phytochrome-interacting factor 6 (PIF6) of Arabidopsis thaliana was exploited for a red light triggered gene expression regulation. Furthermore, ultraviolet B (UVB)—inducible gene expression system were also developed and proven to be efficient in target gene transcription in mammalian cells (Chapter 25 of Gene and Cell Therapy: Therapeutic Mechanisms and Strategies, Fourth Edition CRC Press, January 20^{th}, 2015). Any of the light-inducible promoters described herein may be used to drive expression of the therapeutic protein in the present application.

In some embodiments, the promoter is a light-inducible promoter that is induced by a combination of a light-inducible molecule, and light. For example, a light-cleavable photocaged group on a chemical inducer keeps the inducer inactive, unless the photocaged group is removed through irradiation or by other means. Such light-inducible molecules include small molecule compounds, oligonucleotides, and proteins. For example, caged ecdysone, caged IPTG for use with the lac operon, caged toyocamycin for ribozyme-mediated gene expression, caged doxycycline for use with the Tet-on system, and caged Rapalog for light mediated FKBP/FRB dimerization have been developed (see, for example, Curr Opin Chem Biol. 16(3-4): 292-299 (2012)).

In some embodiments, the promoter is a radiation-inducible promoter, and the inducing condition is radiation, such as ionizing radiation. Radiation inducible promoters are also known in the art to control transgene expression. Alteration of gene expression occurs upon irradiation of cells. For example, a group of genes known as “immediate early genes” can react promptly upon ionizing radiation. Exemplary immediate early genes include, but are not limited to, Erg-1, p2l/WAF-1, GADD45alpha, t-PA, c-Fos, c-Jun, NF-kappaB, and AP1. The immediate early genes comprise radiation responsive sequences in their promoter regions. Consensus sequences CC(A/T)₆GG have been found in the Erg-1 promoter, and are referred to as serum response elements or known as CArG elements. Combinations of radiation induced promoters and transgenes have been intensively studied and proven to be efficient with therapeutic benefits. See, for example, Cancer Biol Ther. 6(7):1005-12 (2007) and Chapter 25 of Gene and Cell Therapy: Therapeutic Mechanisms and Strategies, Fourth Edition CRC Press, January 20^{th}, 2015.

In some embodiments, the promoter is a heat inducible promoter, and the inducing condition is heat. Heat inducible promoters driving transgene expression have also been widely studied in the art. Heat shock or stress protein (HSP) including Hsp90, Hsp70, Hsp60, Hsp40, Hsp10 etc. plays important roles in protecting cells under heat or other physical and chemical stresses. Several heat inducible promoters including heat-shock protein (HSP) promoters and growth arrest and DNA damage (GADD) 153 promoters have been attempted in pre-clinical studies. The promoter of human hsp70B gene, which was first described in 1985 appears to be one of the most highly-efficient heat inducible promoters. Huang et al reported that after introduction of hsp70B-EGFP, hsp70B-TNFalpha and hsp70B-IL12 coding sequences, tumor cells expressed extremely high transgene expression upon heat treatment, while in the absence of heat treatment, the expression of transgenes were not detected. And tumor growth was delayed significantly in the IL12 transgene plus heat treated group of mice in vivo (Cancer Res. 60:3435 (2000)). Another group of scientists linked the HSV-tk suicide gene to hsp70B promoter and test the system in nude mice bearing mouse breast cancer. Mice whose tumor had been administered the hsp70B-HSVtk coding sequence and heat treated showed tumor regression and a significant survival rate as compared to no heat treatment controls (Hum. Gene Ther. 11:2453 (2000)). Additional heat inducible promoters known in the art can be found in, for example, Chapter 25 of Gene and Cell Therapy: Therapeutic Mechanisms and Strategies, Fourth Edition CRC Press, January 20^{th}, 2015. Any of the heat-inducible promoters discussed herein may be used to drive the expression of the therapeutic protein of the present application.

In some embodiments, the promoter is inducible by a redox state. Exemplary promoters that are inducible by redox state include inducible promoter and hypoxia inducible promoters. For instance, Post D E et al developed hypoxia-inducible factor (HIF) responsive promoter which specifically and strongly induce transgene expression in HIF-active tumor cells (Gene Ther. 8: 1801-1807 (2001); Cancer Res. 67: 6872-6881 (2007)).

In some embodiments, the promoter is inducible by the physiological state, such as an endogenous activation signal, of the modified immune cell. In some embodiments, wherein the modified immune cell is a T cell, the promoter is a T cell activation-dependent promoter, which is inducible by the endogenous activation signal of the modified T cell. In some embodiments, the modified T cell is activated by an inducer, such as phorbol myristate acetate (PMA), ionomycin, or phytohaemagglutinin. In some embodiments, the modified T cell is activated by recognition of a tumor antigen on the tumor cells via the engineered receptor (such as CAR, TCR or TAC). In some embodiments, the T cell activation-dependent promoter is an IL-2 promoter. In some embodiments, the T cell activation-dependent promoter is an NFAT promoter. In some embodiments, the T cell activation-dependent promoter is a NF_{K}B promoter.

The heterologous nucleic acid sequences(s) described herein can be present in a heterologous gene expression cassette, which comprises one or more protein-coding sequences and optionally one or more promoters. In some embodiments, the heterologous gene expression cassette comprises a single protein-coding sequence. In some embodiments, the heterologous gene expression cassette comprises two or more protein-coding sequences driven by a single promoter (i.e., polycistronic). In some embodiments, the heterologous gene expression cassette further comprises one or more regulatory sequences (such as 5′UTR, 3′UTR, enhancer sequence, IRES, transcription termination sequence), recombination sites, one or more selection markers (such as antibiotic resistance gene, reporter gene, etc.), signal sequence, or combinations thereof.

In some embodiments, there is provided a vector comprising any one of the nucleic acids encoding the IL-15 polypeptides and/or the engineered receptors described herein. In some embodiments, there is provided a vector comprising a first nucleic acid sequence encoding any one of the IL-15 polypeptides described herein and a second nucleic acid sequence encoding any one of the engineered receptors described herein. In some embodiments, the first nucleic acid sequence encoding the IL-15 polypeptide is fused to the second nucleic acid sequence encoding the engineered receptor via a third nucleic acid sequence encoding a self-cleavable linker, such as P2A, T2A, E2A, or F2A peptide. In some embodiments, the P2A sequence is GSGATNFSLLKQAGDVEENPGP (SEQ ID NO: 28). In some embodiments, there is provided a composition comprising a first vector comprising a first nucleic acid sequence encoding any one of the IL-15 polypeptides described herein, and a second vector comprising a second nucleic acid sequence encoding any one of the engineered receptors described herein.

In some embodiments, there is provided a vector comprising a first nucleic acid sequence encoding a CAR (e.g., a BCMA CAR, CD19 CAR, or GPC3 CAR) and a second nucleic acid sequence encoding a IL-15 polypeptide (e.g., secreted or membrane bound IL-15 polypeptide), wherein the first nucleic acid sequence is fused to the second nucleic acid sequence via a third nucleic acid sequence encoding a self-cleavable linker, such as P2A. In some embodiments, the vector comprises a nucleic acid sequence encoding an amino acid sequence selected from the group consisting of SEQ ID NOs: 29-39, 42-49, 57-66, 75-77, and 82-84.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. In general, a suitable vector contains an origin of replication functional in at least one organism, a promoter sequence, convenient restriction endonuclease sites, and one or more selectable markers. The term “vector” should also be construed to include non-plasmid and non-viral compounds which facilitate transfer of nucleic acid into cells, such as, for example, polylysine compounds, liposomes, and the like.

In some embodiments, the vector is a viral vector. Examples of viral vectors include, but are not limited to, adenoviral vectors, adeno-associated virus vectors, lentiviral vector, retroviral vectors, vaccinia vector, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals.

A number of viral based systems have been developed for gene transfer into mammalian cells. For example, retroviruses provide a convenient platform for gene delivery systems. The heterologous nucleic acid can be inserted into a vector and packaged in retroviral particles using techniques known in the art. The recombinant virus can then be isolated and delivered to the modified immune cell in vitro or ex vivo. A number of retroviral systems are known in the art. In some embodiments, adenovirus vectors are used. In some embodiments, lentivirus vectors are used. In some embodiments, self-inactivating lentiviral vectors are used. For example, self-inactivating lentiviral vectors can be packaged with protocols known in the art. The resulting lentiviral vectors can be used to transduce a mammalian cell (such as human T cells) using methods known in the art.

In some embodiments, the vector is a non-viral vector, such as a plasmid, or an episomal expression vector.

In some embodiments, the vector is an expression vector. “Expression vector” is a construct that can be used to transform a selected host and provides for expression of a coding sequence in the selected host. Expression vectors can for instance be cloning vectors, binary vectors or integrating vectors. Expression comprises transcription of the nucleic acid molecule preferably into a translatable mRNA. Regulatory elements ensuring expression in eukaryotic cells are well known to those skilled in the art. In the case of eukaryotic cells they comprise normally promoters ensuring initiation of transcription and optionally poly-A signals ensuring termination of transcription and stabilization of the transcript. Examples of regulatory elements permitting expression in eukaryotic host cells are AOX1 or GAL1 promoter in yeast or the CMV-, SV40-, RSV-promoter (Rous sarcoma virus), CMV-enhancer, SV40-enhancer or a globin intron in mammalian and other animal cells. Furthermore, depending on the expression system used leader sequences (i.e., signal peptide) capable of directing the polypeptide to a cellular compartment or secreting it into the medium may be added to the coding sequence of the recited nucleic acid sequence and are well known in the art. The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the nucleic acid sequence can encode a fusion protein including an N-terminal identification peptide imparting desired characteristics, e.g., stabilization or simplified purification of expressed recombinant product. Suitable expression vectors are known in the art such as Okayama-Berg cDNA expression vector pcDV1 (Pharmacia), pEF-Neo, pCDM8, pRc/CMV, pcDNA1, pcDNA3 (Invitrogen), pEF-DHFR and pEF-ADA, (Raum et al., Cancer Immunol Immunother (2001) 50(3), 141-150) or pSPORT1 (GIBCO BRL).

### Methods of Preparation

The present application also provides methods of preparing any one of the modified immune cells described herein.

In some embodiments, there is provided a method of producing a modified immune cell, comprising: introducing into a precursor immune cell a first nucleic acid sequence encoding any one of the IL-15 polypeptides described herein. In some embodiments, the precursor immune cell is selected from the group consisting of a cytotoxic T cell, a helper T cell, a natural killer (NK) cell, an NK-T cell, an iNK-T cell, an NK-T like cell, an αβT cell and a γδT cell. In some embodiments, the precursor immune cell is a cytotoxic T cell. In some embodiments, the precursor immune cell is a γδT cell. In some embodiments, the precursor immune cell is a tumor-infiltrating T cell or DC-activated T cell. In some embodiments, the precursor immune cell comprises any one of the engineered receptors described herein. In some embodiments, the method further comprises introducing into the precursor immune cell a second nucleic acid encoding any one of the engineered receptors described herein. In some embodiments, the engineered receptor is a chimeric antigen receptor (CAR). In some embodiments, the engineered receptor is a modified T-cell receptor (TCR). In some embodiments, the engineered receptor is a T-cell antigen coupler (TAC) receptor. In some embodiments, the first nucleic acid sequence and the second nucleic acid sequence are operably linked to the same promoter. In some embodiments, the first nucleic acid sequence and the second nucleic acid sequence are operably linked to separate promoters. In some embodiments, the first nucleic acid and the second nucleic acid are on the same vector. In some embodiments, the first nucleic acid and the second nucleic acid are on separate vectors. In some embodiments, the vector is a viral vector. In some embodiments, the viral vector is selected from the group consisting of an adenoviral vector, an adeno-associated virus vector, a retroviral vector, a lentiviral vector, a herpes simplex viral vector, and derivatives thereof. In some embodiments, the vector is a non-viral vector. In some embodiments, the vector is an episomal expression vector. In some embodiments, the method further comprises isolating or enriching immune cells comprising the first nucleic acid sequence and/or the second nucleic acid sequence. In some embodiments, the method further comprises formulating the modified immune cells with at least one pharmaceutically acceptable carrier.

In some embodiments, there is provided an isolated host cell comprising any one of the nucleic acids or vectors described herein. The host cells may be useful in expression or cloning of the IL-15 polypeptides and/or the engineered receptors, nucleic acids or vectors encoding the IL-15 polypeptides and/or the engineered receptors. Suitable host cells can include, without limitation, prokaryotic cells, fungal cells, yeast cells, or higher eukaryotic cells such as mammalian cells. In some embodiments, the host cells comprise a first vector encoding a first polypeptide and a second vector encoding a second polypeptide. In some embodiments, the host cells comprise a single vector comprising isolated nucleic acids encoding a first polypeptide and a second polypeptide.

The precursor immune cells can be prepared using a variety of methods known in the art. For example, primary immune cells, such as T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, immune cells (such as T cells) can be obtained from a unit of blood collected from an individual using any number of techniques known in the art, such as FICOLL™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS), or a wash solution lacking divalent cations, such as calcium and magnesium. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In some embodiments, primary T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA, and CD45RO cells, can be further isolated by positive or negative selection techniques. For example, in one embodiment, T cells are isolated by incubation with anti-CD3/anti-CD28 (i.e., 3×28)—conjugated beads, such as DYNABEADS® M-450 CD3/CD28 T, for a time period sufficient for positive selection of the desired T cells.

In some embodiments, a T cell population may further be enriched by negative selection using a combination of antibodies directed to surface markers unique to the negatively selected cells. For example, one method involves cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4⁺, CD25⁺, CD62L^{hi}, GITR⁺, and FoxP3⁺. Alternatively, in certain embodiments, T regulatory cells are depleted by anti-C25 conjugated beads or other similar methods of selection.

Methods of introducing vectors or nucleic acids into a host cell (such as a precursor immune cell) are known in the art. The vectors or nucleic acids can be transferred into a host cell by physical, chemical, or biological methods.

Physical methods for introducing the vector(s) or nucleic acid(s) into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001) Molecular Cloning: A Laboratory Manual, Cold Spring Harbor Laboratory, New York. In some embodiments, the vector is introduced into the cell by electroporation.

Biological methods for introducing the vector(s) or nucleic acid(s) into a host cell include the use of DNA and RNA vectors. Viral vectors have become the most widely used method for inserting genes into mammalian, e.g., human cells.

Chemical means for introducing the vector(s) or nucleic acid(s) into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro is a liposome (e.g., an artificial membrane vesicle).

In some embodiments, the transduced or transfected precursor immune cell is propagated ex vivo after introduction of the heterologous nucleic acid(s). In some embodiments, the transduced or transfected precursor immune cell is cultured to propagate for at least about any of 1 day, 2 days, 3 days, 4 days, 5 days, 6 days, 7 days, 10 days, 12 days, or 14 days. In some embodiments, the transduced or transfected precursor immune cell is cultured for no more than about any of 1 day, 2 days, 3 days, 4 days, 5 days, 6 days, 7 days, 10 days, 12 days, or 14 days. In some embodiments, the transduced or transfected precursor immune cell is further evaluated or screened to select the modified immune cell.

Reporter genes may be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al. FEBS Letters 479: 79-82 (2000)).

Other methods to confirm the presence of the heterologous nucleic acid(s) in the precursor immune cell, include, for example, molecular biological assays well known to those of skill in the art, such as Southern and Northern blotting, RT-PCR and PCR; biochemical assays, such as detecting the presence or absence of a particular peptide, e.g., by immunological methods (such as ELISAs and Western blots).

### III. Methods of Treatment

One aspect of the present application relates to methods of treating a disease or condition (e.g., cancer) in an individual, comprising administering to the individual an effective amount of any one of the modified immune cells described herein. The present application contemplates modified immune cells that can be administered either alone or in any combination with another therapy, and in at least some aspects, together with a pharmaceutically acceptable carrier or excipient. In some embodiments, prior to administration, the modified immune cells may be combined with suitable pharmaceutical carriers and excipients that are well known in the art.

In some embodiments, there is provided a method of treating cancer (e.g., solid cancer) in an individual (e.g., human), comprising administering to the individual an effective amount of a pharmaceutical composition comprising a modified immune cell (e.g., an NK cell) and a pharmaceutically acceptable carrier, wherein the modified immune cell comprises a first heterologous nucleic acid sequence encoding an IL-15 polypeptide comprising one or more amino acid substitutions at positions 8, 62, 3 and/or 25, wherein numbering of the amino acid residue positions is according to SEQ ID NO: 1. In some embodiments, the IL-15 polypeptide comprises an amino acid substitution at position 62. In some embodiments, the amino acid substitution at position 62 is selected from the group consisting of T62G, T62I, T62Q, T62V, T62P, T62L, T62A, T62S and T62Y. In some embodiments, the amino acid substitution at position 62 is T62G. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 7. In some embodiments, the IL-15 polypeptide comprises SEQ ID NO: 7. In some embodiments, the IL-15 polypeptide comprises an amino acid substitution at position 8. In some embodiments, the amino acid substitution at position 8 is D8E. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 5. In some embodiments, the IL-15 polypeptide comprises SEQ ID NO: 5. In some embodiments, the IL-15 polypeptide comprises an amino acid substitution at position 3. In some embodiments, the amino acid substitution at position 3 is V3Y. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 78. In some embodiments, the IL-15 polypeptide comprises SEQ ID NO: 78. In some embodiments, the IL-15 polypeptide comprises an amino acid substitution at position 25. In some embodiments, the amino acid substitution at position 25 is selected from the group consisting of L25E and L25F. In some embodiments, the amino acid substitution at position 25 is L25F. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 79. In some embodiments, the IL-15 polypeptide comprises SEQ ID NO: 79. In some embodiments, the IL-15 polypeptide comprises amino acid substitutions at both position 8 and position 62. In some embodiments, the IL-15 polypeptide is secreted. In some embodiments, the IL-15 polypeptide is membrane bound. In some embodiments, the modified immune cell further comprises an engineered receptor, such as a chimeric antigen receptor (CAR), an engineered TCR, or a T-cell antigen coupler (TAC) receptor. In some embodiments, the first nucleic acid sequence and the second nucleic acid sequence are on the same vector or separate vectors. In some embodiments, the first nucleic acid sequence and the second nucleic acid sequence are operably linked to the same promoter or separate promoters. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a method of treating cancer (e.g., solid cancer) in an individual (e.g., human), comprising administering to the individual an effective amount of a pharmaceutical composition comprising a modified immune cell (e.g., an NK cell) and a pharmaceutically acceptable carrier, wherein the modified immune cell comprises a first heterologous nucleic acid sequence encoding an IL-15 polypeptide, which is a fusion protein comprising an IL-15 fragment and a second polypeptide fragment, wherein the IL-15 polypeptide comprises one or more amino acid substitutions at positions 8, 62, 3 and/or 25, wherein numbering of the amino acid residue positions is according to SEQ ID NO: 1. In some embodiments, the IL-15 polypeptide comprises an amino acid substitution at position 62. In some embodiments, the amino acid substitution at position 62 is selected from the group consisting of T62G, T62I, T62Q, T62V, T62P, T62L, T62A, T62S and T62Y. In some embodiments, the amino acid substitution at position 62 is T62G. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 7. In some embodiments, the IL-15 polypeptide comprises SEQ ID NO: 7. In some embodiments, the IL-15 polypeptide comprises an amino acid substitution at position 8. In some embodiments, the amino acid substitution at position 8 is D8E. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 5. In some embodiments, the IL-15 polypeptide comprises SEQ ID NO: 5. In some embodiments, the IL-15 polypeptide comprises an amino acid substitution at position 3. In some embodiments, the amino acid substitution at position 3 is V3Y. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 78. In some embodiments, the IL-15 polypeptide comprises SEQ ID NO: 78. In some embodiments, the IL-15 polypeptide comprises an amino acid substitution at position 25. In some embodiments, the amino acid substitution at position 25 is selected from the group consisting of L25E and L25F. In some embodiments, the amino acid substitution at position 25 is L25F. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 79. In some embodiments, the IL-15 polypeptide comprises SEQ ID NO: 79. In some embodiments, the IL-15 polypeptide comprises amino acid substitutions at both position 8 and position 62. In some embodiments, the second polypeptide fragment is selected from the group consisting of IL-15Rα, an extracellular domain of IL-15Rα, a Sushi domain of IL-15Rα, a transmembrane domain of IL-15Rα, IL-15Rβ, common gamma chain (γc), an engineered receptor (e.g., CAR, TCR or TAC) and combinations thereof. In some embodiments, the modified immune cell further comprises a second heterologous nucleic acid sequence encoding an engineered receptor, such as a CAR, an engineered TCR, or a TAC receptor. In some embodiments, the first nucleic acid sequence and the second nucleic acid sequence are on the same vector or separate vectors. In some embodiments, the first nucleic acid sequence and the second nucleic acid sequence are operably linked to the same promoter or separate promoters. In some embodiments, the modified immune cell is selected from the group consisting of a cytotoxic T cell, a helper T cell, a natural killer (NK) T cell, an iNK-T cell, an NK-T like cell, an αβT cell, a γδT cell, a tumor-infiltrating T cell and a DC-activated T cell.

In some embodiments, there is provided a method of treating cancer (e.g., solid cancer) in an individual (e.g., human), comprising administering to the individual an effective amount of a pharmaceutical composition comprising a modified immune cell (e.g., NK cell) and a pharmaceutically acceptable carrier, wherein the modified immune cell comprises a first heterologous nucleic acid sequence encoding an IL-15 polypeptide comprising a transmembrane domain, wherein the IL-15 polypeptide comprises one or more amino acid substitutions at positions 8, 62, 3 and/or 25, wherein numbering of the amino acid residue positions is according to SEQ ID NO: 1. In some embodiments, the IL-15 polypeptide comprises an amino acid substitution at position 62. In some embodiments, the amino acid substitution at position 62 is selected from the group consisting of T62G, T62I, T62Q, T62V, T62P, T62L, T62A, T62S and T62Y. In some embodiments, the amino acid substitution at position 62 is T62G. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 7. In some embodiments, the IL-15 polypeptide comprises SEQ ID NO: 7. In some embodiments, the IL-15 polypeptide comprises an amino acid substitution at position 8. In some embodiments, the amino acid substitution at position 8 is D8E. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 5. In some embodiments, the IL-15 polypeptide comprises SEQ ID NO: 5. In some embodiments, the IL-15 polypeptide comprises an amino acid substitution at position 3. In some embodiments, the amino acid substitution at position 3 is V3Y. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 78. In some embodiments, the IL-15 polypeptide comprises SEQ ID NO: 78. In some embodiments, the IL-15 polypeptide comprises an amino acid substitution at position 25. In some embodiments, the amino acid substitution at position 25 is selected from the group consisting of L25E and L25F. In some embodiments, the amino acid substitution at position 25 is L25F. In some embodiments, the IL-15 polypeptide comprises an amino acid sequence having at least about 90% (e.g., at least about any one of 91%, 92%, 93%, 94%, 95%, 96%, 97%, 98%, 99%, or higher) sequence identity to SEQ ID NO: 79. In some embodiments, the IL-15 polypeptide comprises SEQ ID NO: 79. In some embodiments, the IL-15 polypeptide comprises amino acid substitutions at both position 8 and position 62. In some embodiments, the transmembrane domain is a transmembrane domain of IL-15Rα. In some embodiments, the IL-15 polypeptide further comprises an intracellular domain. In some embodiments, the IL-15 polypeptide comprises: (a) an antigen-binding domain; (b) an IL-15 fragment; (c) a transmembrane domain; and (d) an intracellular domain. In some embodiments, the antigen-binding domain is at the N-terminus of the IL-15 fragment. In some embodiments, the antigen-binding domain is at the C-terminus of the IL-15 fragment. In some embodiments, the transmembrane domain is a CD4, CD3, CD8α, or CD28 transmembrane domain. In some embodiments, the IL-15 polypeptide further comprises a hinge domain, such as a hinge domain derived from CD8. In some embodiments, the intracellular domain comprises a primary intracellular signaling domain, such as an intracellular signaling domain of CD3ζ. In some embodiments, the intracellular domain comprises a co-stimulatory signaling domain. In some embodiments, the co-stimulatory signaling domain is derived from a co-stimulatory molecule selected from the group consisting of CD27, CD28, 4-1BB, OX40, DAP10, CD30, CD40, CD3, LFA-1, CD2, CD7, LIGHT, NKG2C, B7-H3, ligands of CD83 and combinations thereof. In some embodiments, the modified immune cell further comprises a second heterologous nucleic acid sequence encoding an engineered receptor, such as a CAR, an engineered TCR, or a TAC receptor. In some embodiments, the first nucleic acid sequence and the second nucleic acid sequence are on the same vector or separate vectors. In some embodiments, the first nucleic acid sequence and the second nucleic acid sequence are operably linked to the same promoter or separate promoters. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, the method of treating cancer has one or more of the following biological activities: (1) killing cancer cells; (2) inhibiting proliferation of cancer cells; (3) inducing redistribution of peripheral T cells; (4) inducing immune response in a tumor; (5) reducing tumor size; (6) alleviating one or more symptoms in an individual having cancer; (7) inhibiting tumor metastasis; (8) prolonging survival; (9) prolonging time to cancer progression; (10) preventing, inhibiting, or reducing the likelihood of the recurrence of a cancer; (11) improving quality of life of the individual; (12) facilitating T cell infiltration in tumors, and (13) reducing incidence or burden of preexisting tumor metastasis (such as metastasis to the lymph node). In some embodiments, the method achieves a tumor cell death rate of at least about any of 40%, 50%, 60%, 70%, 80%, 90%, 95%, or more. In some embodiments, the method reduces at least about 10% (including for example at least about any of 20%, 30%, 40%, 60%, 70%, 80%, 90%, or 100%) of the tumor size. In some embodiments, the method inhibits at least about 10% (including for example at least about any of 20%, 30%, 40%, 60%, 70%, 80%, 90%, or 100%) of the metastasis. In some embodiments, the method prolongs the survival of the individual by at least any of 1, 2, 3, 4, 5, 6, 7, 8, 9, 10, 11, 12, 18, 24, or more months. In some embodiments, the method prolongs the time to cancer progression by at least any of 1, 2, 3, 4, 5, 6, 7, 8, 9, 10, 11, 12, 18, 24, or more months.

The methods described herein are suitable for treating a variety of cancers, including both solid cancer and liquid cancer. The methods are applicable to cancers of all stages, including early stage cancer, non-metastatic cancer, primary cancer, advanced cancer, locally advanced cancer, metastatic cancer, or cancer in remission. The methods described herein may be used as a first therapy, second therapy, third therapy, or combination therapy with other types of cancer therapies known in the art, such as chemotherapy, surgery, hormone therapy, radiation, gene therapy, immunotherapy (such as T cell therapy), bone marrow transplantation, stem cell transplantation, targeted therapy, cryotherapy, ultrasound therapy, photodynamic therapy, radio-frequency ablation or the like, in an adjuvant setting or a neoadjuvant setting (i.e., the method may be carried out before the primary/definitive therapy). In some embodiments, the method is used to treat an individual who has previously been treated. In some embodiments, the cancer has been refractory to prior therapy. In some embodiments, the method is used to treat an individual who has not previously been treated.

In some embodiments, the individual has a low tumor burden. Tumor burden for solid tumor can be measured according to the Response Evaluation Criteria in Solid Tumors (RECIST) 1.1 guideline. See, Eisenhauer E A et al., European Journal of Cancer 45 (2009) 228-247. For example, tumor burden can be assessed for measurable tumors at baseline of treatment based on: (1) tumor lesions (e.g., by CT scan, caliper measurement by clinical exam, and/or chest X-ray) and (2) malignant lymph nodes. For example, tumor burden for solid cancer can be quantified as the sum of the diameters of 5 target lesions, with a maximum of 2 per organ. Tumor burden for liquid cancer can be measured as the sum of product diameters of up to 6 index lesions according to Cheson 2007 criteria assessed by a radiologist. See, Cheson B D et al., J. Clin. Oncol., 2007; 25(5): 579-586. In some embodiments, an individual with a low tumor burden has a tumor burden of no more than about any one of 4×10³, 3×10³, 2×10³, 1×10³, 5×10², 2×10², 1×10² or less mm².

In some embodiments, the individual does not experience Grade 3 or Grade 4 adverse side effects after receiving the treatment. Grading of adverse events are according to Commmon Terminology Citeria for Adverse Events v3.0 (CTCAE). In some embodiments, the individual does not experience cytokine storm after receiving the treatment.

The effective amount of the modified immune cells administered in the methods described herein will depend upon a number of factors, such as the particular type and stage of cancer being treated, the route of administrations, the activity of the IL-15 polypeptide and/or the engineered receptors, and the like. Appropriate dosage regimen can be determined by a physician based on clinical factors, including the patient's size, body surface area, age, the particular compound to be administered, sex, time and route of administration, general health, and other drugs being administered concurrently. In some embodiments, that effective amount of the pharmaceutical composition is below the level that induces a toxicological effect (i.e., an effect above a clinically acceptable level of toxicity) or is at a level where a potential side effect can be controlled or tolerated when the pharmaceutical composition is administered to the individual. In some embodiments, the effective amount of the pharmaceutical composition comprises about 10⁵ to about 10¹⁰ modified immune cells.

In some embodiments, the pharmaceutical composition is administered for a single time (e.g. bolus injection). In some embodiments, the pharmaceutical composition is administered for multiple times (such as any of 2, 3, 4, 5, 6, or more times). If multiple administrations, they may be performed by the same or different routes and may take place at the same site or at alternative sites. The pharmaceutical composition may be administered at a suitable frequency, such as from daily to once per year. The optimal dosage and treatment regime for a particular patient can readily be determined by one skilled in the art of medicine by monitoring the patient for signs of disease and adjusting the treatment accordingly.

In some embodiments, the individual to be treated is a mammal. Examples of mammals include, but are not limited to, humans, monkeys, rats, mice, hamsters, guinea pigs, dogs, cats, rabbits, pigs, sheep, goats, horses, cattle and the like. In some embodiments, the individual is a human.

### Pharmaceutical Compositions

Further provided by the present application are pharmaceutical compositions comprising any one of the modified immune cells described herein, and optionally a pharmaceutically acceptable carrier.

The pharmaceutical composition of the present applicant may comprise any number of the modified immune cells. In some embodiments, the pharmaceutical composition comprises a single copy of the modified immune cell. In some embodiments, the pharmaceutical composition comprises at least about any of 1, 10, 100, 1000, 10⁴, 10⁵, 10⁶, 10⁷, 10⁸ or more copies of the modified immune cells. In some embodiments, the pharmaceutical composition comprises a single type of modified immune cell. In some embodiments, the pharmaceutical composition comprises at least two types of modified immune cells, wherein the different types of modified immune cells differ by their cell sources, cell types, expressed chimeric receptors, and/or promoters, etc.

“Carriers” as used herein include pharmaceutically acceptable carriers, excipients, or stabilizers which are nontoxic to the cells or individual being exposed thereto at the dosages and concentrations employed. Often the physiologically acceptable carrier is an aqueous pH buffered solution. Examples of suitable pharmaceutical carriers are well known in the art and include phosphate buffered saline solutions, water, emulsions, such as oil/water emulsions, various types of wetting agents, sterile solutions, etc. Acceptable carriers, excipients, or stabilizers are nontoxic to recipients at the dosages and concentrations employed.

Pharmaceutical compositions comprising such carriers can be formulated by well-known conventional methods. The solvent or diluent is preferably isotonic, hypotonic or weakly hypertonic and has a relatively low ionic strength. Representative examples include sterile water, physiological saline (e.g. sodium chloride), Ringer's solution, glucose, trehalose or saccharose solutions, Hank's solution, and other aqueous physiologically balanced salt solutions (see, for example, the most current edition of Remington: The Science and Practice of Pharmacy, A. Gennaro, Lippincott, Williams&Wilkins).

The pharmaceutical compositions described herein may be administered via any suitable routes. In some embodiments, the pharmaceutical composition is administered parenterally, transdermally (into the dermis), intraluminally, intra-arterially (into an artery), intramuscularly (into muscle), intrathecally or intravenously. In some embodiments, the pharmaceutical composition is administered subcutaneously (under the skin). In some embodiments, the pharmaceutical composition is administered intravenously. In some embodiments, the pharmaceutical composition is administered to the individual via infusion or injection. In some embodiments, the pharmaceutical composition is administered directly to the target site, e.g., by biolistic delivery to an internal or external target site or by catheter to a site in an artery. In some embodiments, the pharmaceutical composition is administered locally, e.g., intratumorally. Administrations may use conventional syringes and needles or any compound or device available in the art capable of facilitating or improving delivery of the active agent(s) in the subject.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishes, electrolyte replenishers (such as those based on Ringer's dextrose), and the like. Preservatives and other additives may also be present such as, for example, antimicrobials, anti-oxidants, chelating agents, and inert gases and the like. In addition, the pharmaceutical composition of the present disclosure might comprise proteinaceous carriers, like, e.g., serum albumin or immunoglobulin, preferably of human origin. Various virus formulation are available in the art either in frozen, liquid form or lyophilized form (e.g. WO98/02522, WO01/66137, WO03/053463, WO2007/056847 and WO2008/114021, etc.). Solid (e.g. dry powdered or lyophilized) compositions can be obtained by a process involving vacuum drying and freeze-drying (see e.g. WO2014/053571). It is envisaged that the pharmaceutical composition of the disclosure might comprise, in addition to the modified immune cells described herein, further biologically active agents, depending on the intended use of the pharmaceutical composition.

In some embodiments, the pharmaceutical composition is suitably buffered for human use. Suitable buffers include without limitation phosphate buffer (e.g. PBS), bicarbonate buffer and/or Tris buffer capable of maintaining a physiological or slightly basic pH (e.g. from approximately pH 7 to approximately pH 9). In some embodiments, the pharmaceutical composition can also be made to be isotonic with blood by the addition of a suitable tonicity modifier, such as glycerol.

In some embodiments, the pharmaceutical composition is contained in a single-use vial, such as a single-use sealed vial. In some embodiments, the pharmaceutical composition is contained in a multi-use vial. In some embodiments, the pharmaceutical composition is contained in bulk in a container.

In some embodiments, the pharmaceutical composition must meet certain standards for administration to an individual. For example, the United States Food and Drug Administration has issued regulatory guidelines setting standards for cell-based immunotherapeutic products, including 21 CFR 610 and 21 CFR 610.13. Methods are known in the art to assess the appearance, identity, purity, safety, and/or potency of pharmaceutical compositions. In some embodiments, the pharmaceutical composition is substantially free of extraneous protein capable of producing allergenic effects, such as proteins of an animal source used in cell culture other than the modified immune cells. In some embodiments, “substantially free” is less than about any of 10%, 5%, 1%, 0.1%, 0.01%, 0.001%, 1 ppm or less of total volume or weight of the pharmaceutical composition. In some embodiments, the pharmaceutical composition is prepared in a GMP-level workshop. In some embodiments, the pharmaceutical composition comprises less than about 5 EU/kg body weight/hr of endotoxin for parenteral administration. In some embodiments, at least about 70% of the modified immune cells in the pharmaceutical composition are alive for intravenous administration. In some embodiments, the pharmaceutical composition has a “no growth” result when assessed using a 14-day direct inoculation test method as described in the United States Pharmacopoeia (USP). In some embodiments, prior to administration of the pharmaceutical composition, a sample including both the modified immune cells and the pharmaceutically acceptable excipient should be taken for sterility testing approximately about 48-72 hours prior to the final harvest (or coincident with the last re-feeding of the culture). In some embodiments, the pharmaceutical composition is free of mycoplasma contamination. In some embodiments, the pharmaceutical composition is free of detectable microbial agents. In some embodiments, the pharmaceutical composition is free of communicable disease agents, such as HIV type I, HIV type II, HBV, HCV, Human T-lymphotropic virus, type I; and Human T-lymphotropic virus, type II.

### IV. Kits and Articles of Manufacture

Also provided are kits, unit dosages, and articles of manufacture comprising any one of the modified immune cells, or the compositions (e.g. pharmaceutical composition) described herein. In some embodiments, a kit is provided which contains any one of the pharmaceutical compositions described herein and preferably provides instructions for its use. In some embodiments, the kit, in addition to the modified immune cell, further comprises a second cancer therapy, such as chemotherapy, hormone therapy, and/or immunotherapy. The kit(s) may be tailored to a particular cancer for an individual and comprise respective second cancer therapies for the individual.

The kits may contain one or more additional components, such as containers, reagents, culturing media, inducers, cytokines, buffers, antibodies, and the like to allow propagation or induction of the modified immune cell. The kits may also contain a device for local administration (such as intratumoral injection) of the pharmaceutical composition to a tumor site.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like. Some components of the kits may be packaged either in aqueous media or in lyophilized form.

The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder (such as cancer) described herein, and may have a sterile access port (for example the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g., from 2-6 administrations) of the reconstituted formulation. Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical composition and instructions for use, packaged in quantities sufficient for storage and use in pharmacies, for example, hospital pharmacies and compounding pharmacies.

### EXEMPLARY EMBODIMENTS

Embodiment 1. A modified immune cell comprising a first heterologous nucleic acid sequence encoding an IL-15 polypeptide comprising one or more amino acid substitutions at positions 8 and/or 62, wherein numbering of the amino acid residue positions is according to SEQ ID NO: 1.

Embodiment 2. The modified immune cell of embodiment 1, wherein the IL-15 polypeptide comprises an amino acid substitution at position 62.

Embodiment 3. The modified immune cell of embodiment 2, wherein the IL-15 polypeptide comprises an amino acid residue selected from the group consisting of Glycine (G), Isoleucine (I), Glutamine (Q), Valine (V), Proline (P), Leucine (L), Alanine (A), Serine (S) and Tyrosine (Y) at position 62.

Embodiment 4. The modified immune cell of embodiment 2 or 3, wherein the amino acid substitution at position 62 is selected from the group consisting of T62G, T62I, T62Q, T62V, T62P, T62L, T62A, T62S and T62Y.

Embodiment 5. The modified immune cell of embodiment 4, wherein the amino acid substitution at position 62 is T62G.

Embodiment 6. The modified immune cell of any one of embodiments 2-5, wherein the IL-15 polypeptide comprises an amino acid sequence having at least 90% sequence identity to the amino acid sequence of SEQ ID NO: 7.

Embodiment 7. The modified immune cell of any one of the preceding embodiments, wherein the IL-15 polypeptide comprises an amino acid substitution at position 8.

Embodiment 8. The modified immune cell of embodiment 7, wherein the IL-15 polypeptide comprises an amino acid residue Glutamic acid (E) at position 8.

Embodiment 9. The modified immune cell of embodiment 8, wherein the amino acid substitution at position 8 is D8E.

Embodiment 10. The modified immune cell of embodiment 8 or 9, wherein the IL-15 polypeptide comprises the amino acid sequence having at least 90% sequence identity to the amino acid sequence of SEQ ID NO: 5.

Embodiment 11. The modified immune cell comprising a first heterologous nucleic acid sequence encoding an IL-15 polypeptide comprising one or more amino acid substitutions at positions 3 and/or 25, wherein numbering of the amino acid residue positions is according to SEQ ID NO: 1.

Embodiment 12. The modified immune cell of embodiment 11, wherein the amino acid substitution at position 3 is V3Y and/or the amino acid substitution at position 25 is L25F.

Embodiment 13. The modified immune cell of embodiment 12, wherein the IL-15 polypeptide comprises the amino acid sequence having at least 90% sequence identity to the amino acid sequence of SEQ ID NO: 78 or 79.

Embodiment 14. The modified immune cell of any one of the preceding embodiments, wherein the one or more amino acid substitutions reduce affinity of the IL-15 polypeptide to IL-15Rβ compared to an IL-15 polypeptide that does not comprise the one or more amino acid substitutions.

Embodiment 15. A modified immune cell comprising a first heterologous nucleic acid sequence encoding an IL-15 polypeptide that induces secretion of an inflammatory cytokine by the modified immune cell at a level that is least 50% lower than that by a modified immune cell comprising a heterologous nucleic acid sequence encoding a wildtype IL-15 polypeptide.

Embodiment 16. The modified immune cell of any one of the preceding embodiments, wherein the IL-15 polypeptide is secreted.

Embodiment 17. The modified immune cell of any one of embodiments 1-16, wherein the IL-15 polypeptide is membrane bound.

Embodiment 18. The modified immune cell of embodiment 17, wherein the IL-15 polypeptide comprises a glycosylphosphatidylinositol (GPI)—anchoring peptide sequence.

Embodiment 19. The modified immune cell of embodiment 17, wherein the IL-15 polypeptide comprises a transmembrane domain.

Embodiment 20. The modified immune cell of embodiment 17, wherein the IL-15 polypeptide comprises a membrane anchoring domain.

Embodiment 21. The modified immune cell of any one of the preceding embodiments, wherein the IL-15 polypeptide is a fusion protein comprising an IL-15 fragment fused to a second polypeptide fragment.

Embodiment 22. The modified immune cell of embodiment 21, wherein the second polypeptide fragment is selected from the group consisting of TL-15Rα, an extracellular domain of IL-15Rα, a Sushi domain of IL-15Rα, a transmembrane domain of IL-15Rα, IL-15Rβ, and common gamma chain (γc).

Embodiment 23. The modified immune cell of embodiment 22, wherein the second polypeptide fragment comprises an amino acid sequence selected from the group consisting of SEQ ID NOs: 50-55.

Embodiment 24. The modified immune cell of embodiment 17, wherein the IL-15 polypeptide comprises: (a) an antigen-binding domain; (b) an IL-15 fragment; (c) a transmembrane domain; and (d) an intracellular domain.

Embodiment 25. The modified immune cell of any one of embodiments 1-23, wherein the modified immune cell comprises a second heterologous nucleic acid sequence encoding an engineered receptor.

Embodiment 26. The modified immune cell of embodiment 25, wherein the engineered receptor is a chimeric antigen receptor (CAR).

Embodiment 27. The modified immune cell of embodiment 26, wherein the CAR is a BCMA CAR, a CD19 CAR, or a GPC3 CAR.

Embodiment 28. The modified immune cell of embodiment 25, wherein the engineered receptor is a modified T-cell receptor (TCR).

Embodiment 29. The modified immune cell of embodiment 25, wherein the engineered receptor is a T-cell antigen coupler (TAC) receptor.

Embodiment 30. The modified immune cell of any one of embodiments 25-29, wherein the first nucleic acid sequence and the second nucleic acid sequence are operably linked to the same promoter.

Embodiment 31. The modified immune cell of any one of embodiments 25-29, wherein the first nucleic acid and the second nucleic acid are operably linked to separate promoters.

Embodiment 32. The modified immune cell of any one of the preceding embodiments, wherein the modified immune cell is selected from the group consisting of a cytotoxic T cell, a helper T cell, a natural killer (NK) cell, an NK-cell, an iNK-T cell, an NK-T like cell, an αβT cell and a γδT cell.

Embodiment 33. The modified immune cell of embodiment 32, wherein the modified immune cell is an NK cell.

Embodiment 34. The modified immune cell of embodiment 32, wherein the modified immune cell is a cytotoxic T cell.

Embodiment 35. The modified immune cell of any one of the preceding embodiments, wherein the modified immune cell has reduced toxicity in vivo when administered to an individual compared to a modified immune cell that does not comprise the first heterologous nucleic acid encoding the IL-15 polypeptide.

Embodiment 36. A method of producing a modified immune cell, comprising: introducing into a precursor immune cell a first nucleic acid sequence encoding an IL-15 polypeptide comprising one or more amino acid substitutions at positions 8 and/or 62, wherein numbering of the amino acid residue positions is according to SEQ ID NO: 1.

Embodiment 37. The method of embodiment 36, wherein the precursor immune cell is selected from the group consisting of a cytotoxic T cell, a helper T cell, an NK cell, an NK-T cell, an iNK-T cell, an NK-T like cell, an αβT cell and a γδT cell.

Embodiment 38. The method of embodiment 36 or 37, wherein the precursor immune cell comprises an engineered receptor.

Embodiment 39. The method of embodiment 36 or 37, further comprising introducing into the precursor immune cell a second nucleic acid encoding an engineered receptor.

Embodiment 40. The method of embodiment 38 or 39, wherein the engineered receptor is a chimeric antigen receptor (CAR), a modified T-cell receptor (TCR), or a T-cell antigen coupler (TAC) receptor.

Embodiment 41. The method of embodiment 39 or 40, wherein the first nucleic acid sequence and the second nucleic acid sequence are on the same vector.

Embodiment 42. The method of embodiment 41, wherein the vector is a viral vector.

Embodiment 43. The method of embodiment 42, wherein the viral vector is selected from the group consisting of an adenoviral vector, an adeno-associated virus vector, a retroviral vector, a lentiviral vector, a herpes simplex viral vector, and derivatives thereof.

Embodiment 44. The method of any one of embodiments 36-43, further comprising isolating or enriching immune cells comprising the first and/or the second nucleic acid sequence.

Embodiment 45. A modified immune cell produced by the method of any one of embodiments 36-44.

Embodiment 46. A pharmaceutical composition comprising the modified immune cell of embodiments 1-35 and 45, and a pharmaceutically acceptable carrier.

Embodiment 47. A method of treating a disease in an individual, comprising administering to the individual an effective amount of the pharmaceutical composition of embodiment 46.

Embodiment 48. The method of embodiment 47, wherein the disease is cancer.

Embodiment 49. The method of embodiment 48, wherein the individual has a low tumor burden.

Embodiment 50. The method of any one of embodiments 47-49, wherein the method does not result in cytokine storm in the individual.

Embodiment 51. The method of any one of embodiments 47-50, wherein the individual is human.

Embodiment 52. A method of reducing cytokine storm in an individual receiving treatment with an immune cell comprising an engineered receptor, comprising: (a) introducing to the immune cell a heterologous nucleic acid sequence encoding an IL-15 polypeptide comprising one or more amino acid substitutions at positions 8 and/or 62, wherein numbering of the amino acid residue positions is according to SEQ ID NO: 1, thereby providing a modified immune cell; and (b) administering to the individual an effective amount of the modified immune cell.

Embodiment 53. An engineered IL-15 polypeptide comprising amino acid substitution D8E, T62G, V3Y and/or L25F, wherein numbering of the amino acid residue positions is according to SEQ ID NO: 1.

Embodiment 54. The engineered IL-15 polypeptide of embodiment 53, comprising an amino acid sequence having at least about 90% sequence identity to an amino acid sequence selected from the group consisting of SEQ ID NOs: 5 7, 78 and 79.

### EXAMPLES

The examples below are intended to be purely exemplary of the disclosure and should therefore not be considered to limit the disclosure in any way. The following examples and detailed description are offered by way of illustration and not by way of limitation.

### Example 1: Preparation of CAR-NK Cells Expressing Exogenously Introduced Wildtype or Mutant IL-15

This example shows the construction of exemplary armored CAR-NK cells expressing exogenously introduced IL-15. In particular, this example shows the construction of wildtype or mutant IL-15 armored BCMA CAR-NK cells and wildtype or mutant IL-15 armored CD19 CAR-NK cells.

### 1.1. Construction of Chimeric Antigen Receptors (CARs)

To construct BCMA CAR or CD19 CAR, a CAR backbone sequence encoding a CAR backbone polypeptide comprising from the N-terminus to the C-terminus: a CD8α hinge domain (SEQ ID NO: 18), a CD8α transmembrane domain (SEQ ID NO: 19), a CD137 co-stimulatory signaling domain (SEQ ID NO: 20), a CD3ζ (primary intracellular signaling domain (SEQ ID NO: 21) and, operatively, an IL-15 armored design sequence (i.g., one sequence selected from SEQ ID NO: 2 to SEQ ID NO: 17, and SEQ ID NO: 78 and SEQ ID NO: 79) was chemically synthesized and cloned into a pre-modified retroviral vector (MSCV vector), downstream and operably linked to a constitutive hEF1α promoter for in vitro transcription. Transient retroviral supernatants were produced refer as exemplified in Blood (2006) 108 (12): 3890-3897. The sequences of the examplary CAR constructs are shown below.

BCMA CAR amino acid sequence
SEQ ID NO: 26
MALPVTALLLPLALLLHAARPAVQLVESGGGLVQAGDSLRLTCTASGRA
FSTYFMAWFRQAPGKEREFVAGIAWSGGSTAYADSVKGRFTISRDNAKN
TVYLQMNSLKSEDTAVYYCASRGIEVEEFGAWGQGTQVTVSSGGGGSQV
QLEESGGGSVQAGGSLRLSCAYTYSTYSNYYMGWFREAPGKARTSVAII
SSDTTITYKDAVKGRFTISKDNAKNTLYLQMNSLKPEDSAMYRCAAWTS
DWSVAYWGQGTQVTVSSTSTTTPAPRPPTPAPTIASQPLSLRPEACRPA
AGGAVHTRGLDFACDIYIWAPLAGTCGVLLLSLVITLYCKRGRKKLLYI
FKQPFMRPVQTTQEEDGCSCRFPEEEEGGCELRVKFSRSADAPAYQQGQ
NQLYNELNLGRREEYDVLDKRRGRDPEMGGKPRRKNPQEGLYNELQKDK
MAEAYSEIGMKGERRRGKGHDGLYQGLSTATKDTYDALHMQALPPR
sIL-15 wt armored BCMA CAR amino acid sequence
SEQ ID NO: 29
MALPVTALLLPLALLLHAARPAVOLVESGGGLVQAGDSLRLTCTASGRA
FSTYFMAWFRQAPGKEREFVAGIAWSGGSTAYADSVKGRFTISRDNAKN
TVYLQMNSLKSEDTAVYYCASRGIEVEEFGAWGQGTQVTVSSGGGGSQV
QLEESGGGSVQAGGSLRLSCAYTYSTYSNYYMGWFREAPGKARTSVAII
SSDTTITYKDAVKGRFTISKDNAKNTLYLQMNSLKPEDSAMYRCAAWTS
DWSVAYWGQGTQVTVSSTSTTTPAPRPPTPAPTIASQPLSLRPEACRPA
AGGAVHTRGLDFACDIYIWAPLAGTCGVLLLSLVITLYCKRGRKKLLYI
FKQPFMRPVQTTQEEDGCSCRFPEEEEGGCELRVKFSRSADAPAYQQGQ
NQLYNELNLGRREEYDVLDKRRGRDPEMGGKPRRKNPQEGLYNELQKDK
MAEAYSEIGMKGERRRGKGHDGLYQGLSTATKDTYDALHMQALPPRGSG
ATNFSLLKQAGDVEENPGPMRISKPHLRSISIQCYLCLLLNSHFLTEAG
IHVFILGCFSAGLPKTEANWVNVISDLKKIEDLIQSMHIDATLYTESDV
HPSCKVTAMKCFLLELQVISLESGDASIHDTVENLIILANNSLSSNGNV
TESGCKECEELEEKNIKEFLQSFVHIVQMFINTS

### 1.2. Construction of CAR-Natural Killer (NK) Cells

### NK Expansion

Human peripheral blood mononuclear cells (PBMCs) were purchased from HemaCare Corporation. PBMCs were thawed and cultured with a K562 genetically modified membrane bound IL-15 and a 4-1BB ligand (i.e., K562-mb15-41BBL). The cells were expanded using stem cell growth medium (SCGM; Cell Genix, Freiburg) supplemented with 50 IU of IL-2/mL of culture medium. After 7 days of culture, cells were collected and purified using anti-CD3 dynabeads (Miltenyi, Cat #11365D). The resulting NK cells were cultured and expanded in SCGM medium supplemented with 50 IU of IL-2/mL of culture medium.

### Virus Transduction of NK Cells

NK cells were collected and suspended at a concentration of 0.25×10⁶ cells in 2 mL of RPMI-1640 medium. Retrovirus supernatant was added to the NK cells, and the cells were incubated at 37° C. overnight. Following incubation, the cells were pelleted by centrifugation and medium was replace with fresh SCGM with 200 IU of IL-2/mL culture medium. Transduced NK cells were cultured, and used for experiments following their expansion for 12 to 20 days.

### Example 2: In Vitro Screening of Rationale-Designed Mutant IL-15 Armored CAR-NK Cells

This example shows comparable in vitro anti-tumor activity of mutant secreted IL-15 (i.g., “sIL-15 m6” or “sIL-15 m4”) armored CAR-NK cells compared with wildtype secreted IL-15 armored CAR-NK cells (i.e., “sIL-15 wt”).

Affinity measurement of IL-15 muteins to IL-15Rα and IL-2Rβ

The IL-15 receptor consists of three polypeptides, the type-specific IL-15R alpha (“IL-15R □”), the common IL-2/IL-15Rbeta (“IL-15Rβ” or “IL-2Rβ”), and the common gamma chain (“γC” or “gC”). The binding domain of IL-15, responsible for the binding of IL-15 to IL-15 alpha and beta receptors was analyzed. Several single residue mutations were made, and the in vitro binding affinity of each mutant IL-15 polypeptide to the IL-15Rα and IL-15Rβ was performed.

Briefly, HEK293 cells were pelleted and the crude IL-15 mutein supernatants were used for affinity measurement by Surface Plasmon Resonance (SPR). The experiment was performed on a Biacore T200 SPR biosensor (GE Healthcare) at room temperature. The anti-Avi tag sensor chips were prepared at 25° C. with a running buffer of 10 mM HEPES, 150 mM NaCl, 3 mM EDTA, and 0.005% (v/v) Tween-20, pH 7.4. All surfaces of a Biacore CM5 sensor chip were activated with a 1:1 (v/v) mixture of 400 mM EDC and 100 mM NHS for 7 minutes, at a flow rate of 10 μL/min. An anti-Avi reagent (Genscript, Cat. No: A00674-200) was diluted to 30 μg/mL in 10 mM sodium acetate (pH 5.0) and injected on all flow cells for 7 minutes at 10 μL/min. All flow cells were blocked with 1 M Ethanolamine-HCl, pH 8.5 for 7 minutes at 10 μL/min.

The affinity determination of all mutein supernatants was performed at 25° C. on Biacore T200 using a running buffer of 10 mM HEPES, 150 mM NaCl, 3 mM EDTA, and 0.005% (v/v) Tween-20, pH 7.4. Avi-tagged IL-15 muteins were captured on flow cells 2, 3 and 4 at a flow rate of 10 μL/min for 30 seconds. The flow cell 1 was used as a reference surface. Following capture of IL-15 muteins, analytes (human IL-15Rα protein concentration ranging from 0.15625 nM to 1280 nM or human IL-2Rβ concentration ranging from 0.625 nM to 1280 nM) was injected at a flow rate of 30 μL/min in all flow cells for 100 seconds. After each analyte injection, dissociation was monitored for 600 seconds (i.e., human IL-15Rα protein as analyte) or 300 seconds (i.e., human IL-2Rβ protein as analyte), followed by regeneration of all flow cells with three 15-second injections of 10 mM Glycine-HCl, pH2.0. Sensorgrams of buffer cycles were collected for double-referencing purposes (see, e.g., Myszka DG. 1999. Journal of molecular recognition 12: 279-284). For kinetic analysis, the double-referenced sensorgrams were fit globally to a simple 1:1 Langmuir binding model using Biacore T200 Evaluation Software version 3.0.

The kinetics and affinity parameters for examplary muteins are shown in Tables 3-4 below.

TABLE 3
Binding affinity of IL-15 muteins to IL-15Rα

| | human IL-15R alpha-His | | | | | |
|---|---|---|---|---|---|---|
| | Capture | Binding | | | | Affinity change |
| mutein | Level | Level | ka (1/Ms) | kd (1/s) | KD (M) | (muteins/WT) |
| m1 | 62.2 | 19.5 | >1.0E+07* | 1.7E−05 | <1.7E−12* | ~0.14 |
| m2 | 388.7 | 63.8 | 3.2E+04 | 2.3E−04 | 7.0E−09 | ~583 |
| m3 | 564.3 | 24.6 | 3.0E+06 | 1.0E−01 | 3.4E−08 | ~2833 |
| m4 | 57.4 | 25.3 | >1.0E+07* | 3.0E−05 | <3.0E−12* | ~0.25 |
| m5 | 119.4 | 37.2 | >1.0E+07* | 1.0E−04 | <1.0E−11* | ~8.83 |
| m6 | 213.8 | 20.7 | >1.0E+07* | 8.3E−05 | <8.3E−12* | ~0.78 |
| m7 | 87.9 | 33.2 | >1.0E+07* | 4.2E−05 | <4.2E−12* | ~0.35 |
| m8 | 420.1 | 44 | 8.0E+06 | 5.4E−02 | 6.7E−09 | ~558 |
| WT | 61.7 | 12.1 | >1.0E+07* | 1.2E−04 | <1.2E−11* | / |

*out off Biacore T200 limitation

TABLE 4
Binding affinity of IL-15 muteins to IL-15Rβ

| | human IL-15R beta-His | | | | | |
|---|---|---|---|---|---|---|
| | Capture | Binding | | | | Affinity change |
| mutein | Level | Level | ka (1/Ms) | kd (1/s) | KD (M) | (muteins/WT) |
| m1 | 207.4 | 50.3 | 9.7E+05 | 8.0E−03 | 8.3E−09 | 0.8 |
| m2 | 761.2 | 5.3 | 2.7E+04 | 7.7E−02 | 2.9E−06 | 285.5 |
| m3 | 218.9 | 46.2 | 8.6E+04 | 7.4E−03 | 8.6E−08 | 8.5 |
| m4 | 1057.7 | 3.6 | 8.4E+03 | 1.7E−03 | 2.0E−07 | 20 |
| m5 | 153.1 | 30.9 | 6.7E+05 | 2.1E−03 | 3.1E−09 | 0.3 |
| m6 | 608 | 14.2 | 5.6E+05 | 8.2E−02 | 1.5E−07 | 14.4 |
| m7 | 613.8 | 22.2 | 1.6E+05 | >1.0E−01* | >6.7E−07* | >67 |
| m8 | 240.8 | 41.2 | 8.2E+05 | 9.2E−03 | 1.1E−08 | 1.1 |
| WT | 199.8 | 33.1 | 8.2E+05 | 8.3E−03 | 1.0E−08 | / |

*out off Biacore T200 limitation
Screening of CAR-NK cells armored with IL-15 muteins

Some mutants were selected for expression in armored CAR-NK cells, namely, m1, m2, m3, m4, m5, m6, m7, m8, m17 and m18, which comprise amino acid substitutions A23L, L25E, Y26G, D8E, D61E, T62G, T62I, E89K, V3Y and L25F, respectively, on human wildtype IL-15 (SEQ TD NO: 1). Table 1 shows the amino acid sequences of the IL-15 muteins. Table 5 shows the constructs of IL-15 armored CAR-NK cells expressing wildtype and mutant sIL-15. The CAR-NK cells were generated using the methods described in Example 1.

TABLE 5
Structures of IL-15 armored CAR

| | | | Amino |
|---|---|---|---|
| | | | Acid |
| CAR Name | IL-15 form | Expression Structure | Sequence |
| sIL-15 wt armored | wild type hIL-15 | CD8αSP-VHH1-VHH2-CD8αhinge&TM- | SEQ ID |
| BCMA CAR | | 4-1BB-CD3zeta -P2A-SP-hIL15 | NO: 29 |
| sIL-15 wt armored | wild type hIL-15 | CD8αSP-CD19 scFv-CD8αhinge&TM-4- | SEQ ID |
| CD19 CAR | | 1BB-CD3zeta-P2A-SP-hIL15 | NO: 30 |
| sIL-15 m1 armored | IL15 mutein 1 | CD8αSP-VHH1-VHH2-CD8αhinge&TM- | SEQ ID |
| BCMA CAR | | 4-1BB -CD3zeta -P2A-SP-IL15 mutein 1 | NO: 31 |
| sIL-15 m2 armored | IL15 mutein 2 | CD8αSP-VHH1-VHH2-CD8αhinge&TM- | SEQ ID |
| BCMA CAR | | 4-1BB -CD3zeta -P2A-SP-IL15 mutein 2 | NO: 32 |
| sIL-15 m3 armored | IL15 mutein 3 | CD8αSP-VHH1-VHH2-CD8αhinge&TM- | SEQ ID |
| BCMA CAR | | 4-1BB-CD3zeta -P2A-SP-IL15 mutein 3 | NO: 33 |
| sIL-15 m4 armored | IL15 mutein 4 | CD8αSP-VHH1-VHH2-CD8αhinge&TM- | SEQ ID |
| BCMA CAR | | 4-1BB-CD3zeta -P2A-SP-IL15 mutein 4 | NO: 34 |
| sIL-15 m5 armored | IL15 mutein 5 | CD8αSP-VHH1-VHH2-CD8αhinge&TM- | SEQ ID |
| BCMA CAR | | 4-1BB-CD3zeta -P2A-SP-IL15 mutein 5 | NO: 35 |
| sIL-15 m6 armored | IL15 mutein 6 | CD8αSP-VHH1-VHH2-CD8αhinge&TM- | SEQ ID |
| BCMA CAR | | 4-1BB-CD3zeta -P2A-SP-IL15 mutein 6 | NO: 36 |
| sIL-15 m7 armored | IL15 mutein 7 | CD8αSP-VHH1-VHH2-CD8αhinge&TM- | SEQ ID |
| BCMA CAR | | 4-1BB-CD3zeta -P2A-SP-IL15 mutein 7 | NO: 37 |
| sIL-15 m8 armored | IL15 mutein 8 | CD8αSP-VHH1-VHH2-CD8αhinge&TM- | SEQ ID |
| BCMA CAR | | 4-1BB -CD3zeta -P2A-SP-IL15 mutein 8 | NO: 38 |
| sIL-15 m9 armored | IL15 mutein 9 | CD8αSP-VHH1-VHH2-CD8αhinge&TM- | SEQ ID |
| BCMA CAR | | 4-1BB -CD3zeta -P2A-SP-IL15 mutein 9 | NO: 42 |
| sIL-15 m10 | IL15 mutein 10 | CD8αSP-VHH1-VHH2-CD8αhinge&TM- | SEQ ID |
| armored BCMA | | 4-1BB -CD3zeta -P2A-SP-IL15 mutein 10 | NO: 43 |
| CAR | | | |
| sIL-15 m11 | IL15 mutein 11 | CD8αSP-VHH1-VHH2-CD8αhinge&TM- | SEQ ID |
| armored BCMA | | 4-1BB -CD3zeta -P2A-SP-IL15 mutein 11 | NO: 44 |
| CAR | | | |
| sIL-15 m12 | IL15 mutein 12 | CD8αSP-VHH1-VHH2-CD8αhinge&TM- | SEQ ID |
| armored BCMA | | 4-1BB -CD3zeta -P2A-SP-IL15 mutein 12 | NO: 45 |
| CAR | | | |
| sIL-15 m13 | IL15 mutein 13 | CD8αSP-VHH1-VHH2-CD8αhinge&TM- | SEQ ID |
| armored BCMA | | 4-1BB -CD3zeta -P2A-SP-IL15 mutein 13 | NO: 46 |
| CAR | | | |
| sIL-15 m14 | IL15 mutein 14 | CD8αSP-VHH1-VHH2-CD8αhinge&TM- | SEQ ID |
| armored BCMA | | 4-1BB -CD3zeta -P2A-SP-IL15 mutein 14 | NO: 47 |
| CAR | | | |
| sIL-15 m15 | IL15 mutein 15 | CD8αSP-VHH1-VHH2-CD8αhinge&TM- | SEQ ID |
| armored BCMA | | 4-1BB -CD3zeta -P2A-SP-IL15 mutein 15 | NO: 48 |
| CAR | | | |
| sIL-15 m16 | IL15 mutein 16 | CD8αSP-VHH1-VHH2-CD8αhinge&TM- | SEQ ID |
| armored BCMA | | 4-1BB -CD3zeta -P2A-SP-IL15 mutein 16 | NO: 49 |
| CAR | | | |
| mbIL-15 wt | hIL15-IL15RaTM | CD8αSP-VHH1-VHH2-CD8αhinge&TM- | SEQ ID |
| armored BCMA | | 4-1BB -CD3zeta-P2A-SP-hIL15- | NO: 39 |
| CAR | | IL15RaTM | |
| sIL-15 wt armored | wild type hIL-15 | CD8αSP-GPC3 scFv -CD8αhinge&TM-4- | SEQ ID |
| GPC3 CAR | | 1BB-CD3zeta -P2A-SP-hIL15 | NO: 82 |
| sIL-15 m17 | IL15 mutein 17 | CD8αSP-GPC3 scFv -CD8αhinge&TM-4- | SEQ ID |
| armored GPC3 | | 1BB-CD3zeta -P2A-SP- IL15 mutein 17 | NO: 83 |
| CAR | | | |
| sIL-15 m18 | IL15 mutein 18 | CD8αSP-GPC3 scFv -CD8αhinge&TM-4- | SEQ ID |
| armored GPC3 | | 1BB-CD3zeta -P2A-SP- IL15 mutein 18 | NO: 84 |
| CAR | | | |

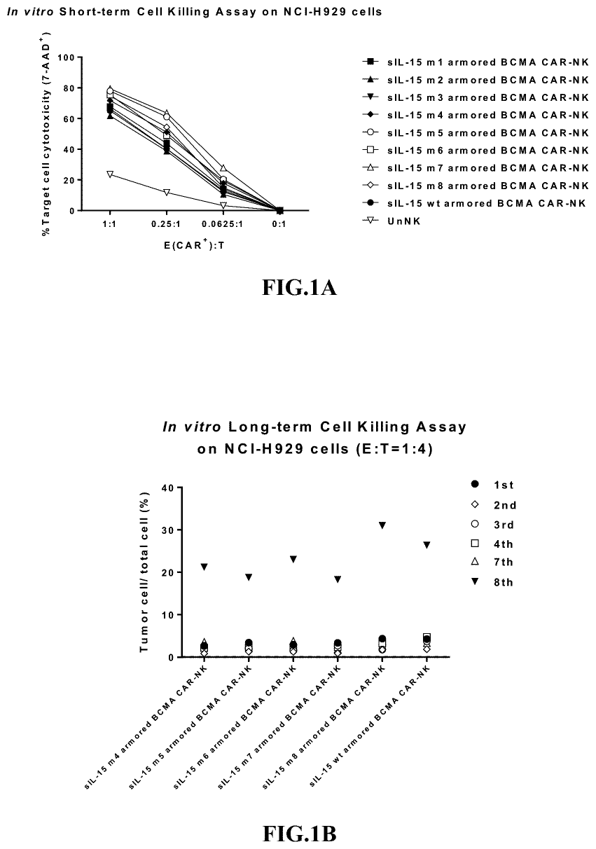

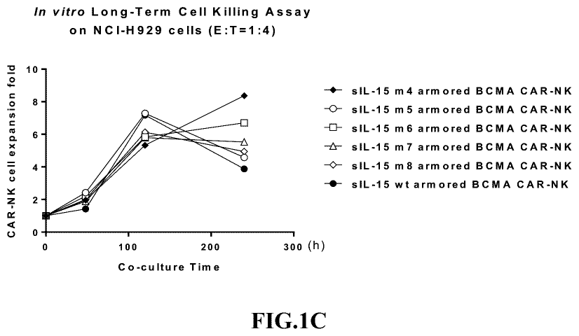

The mutant IL-15 armored CAR-NK cells were tested in vitro for cytotoxicity in a short-term (FIG. 1A) and long-term (FIG. 1B) cell killing assay. FIG. 1A shows in vitro short-term (4 hours) killing of BCMA-positive target, NCI-H929 cells, by the mutant IL-15 armored BCMA CAR-NK cells (E:T=1:1, 0.25:1, 0.0625:1). All mutant IL-15 (e.g., m1-m8) armored BCMA CAR-NIK cells showed potent anti-tumor efficacy in the short-term killing assay compared to untransduced NK cell (i.e., “UnNK”) controls. The percentage of cytotoxicity on target cells was calculated by 7-AAD+%: 7-AAD+cells/Target cells×100%.

FIG. 1B shows the in vitro long-term killing of the mutant IL-15 (e.g., m4 to m8) armored BCMA CAR-NK cells on NCI-H929 cells (E:T=1:4). In each run stimulation, tumor cells were added into NIK cells for co-culture about 24 hours to 48 hours, and after co-culture, cells were collected for further analyzed by flow cytometry. Runs were repeated for a total of 8 antigen stimulations. As shown in FIG. 1B, m4, m5, m6 and m7 mutant IL-15 armored BCMA CAR-NK cells showed better anti-tumor efficacy compared to sIL-15 wt armored BCMA CAR-NK cells. During the stimulation with tumor cells, m4 and m6 mutant IL-15 armored BCMA CAR-NK additionally showed better expansion (FIG. 1C). NK cells expressing wild type hIL-15 (hIL-15-P2A-EGFP, SEQ ID NO: 56) were used as a control.

### Example 3: In Vivo Toxicity of Wildtype IL-15 Armored BCMA CAR-NK Cells

This example shows that toxicity is observed in wildtype secreted IL-15 (i.e., “sIL-15 wt”) armored CAR-NK cells, independent of BCMA target and CAR expression, in a NCG mouse model (NCI-H929-luc model) with a multiple myeloma tumor xenografts.

BCMA CAR constructs were prepared as described in Example 1. To create the tumor xenograft, NCG mice were injected intravenously with NCI-H929-Luc cells (1×10⁶ cells/mouse, BCMA positive multiple myeloma cell line, #ATCC CRL-9068™, transduced with Luciferase). Ten to fourteen days later, tumor engrafted mice were treated with sIL-15 wt armored CD19 CAR-NK cells, sIL-15 wt armored BCMA CAR-NK cells, or un-transduced NK cells comprising human IL-15 at 0.5 μg/mouse (i.e., hIL-15 intraperitoneal injection or “UnNK cell, i.v., IL-15, i.p.”).

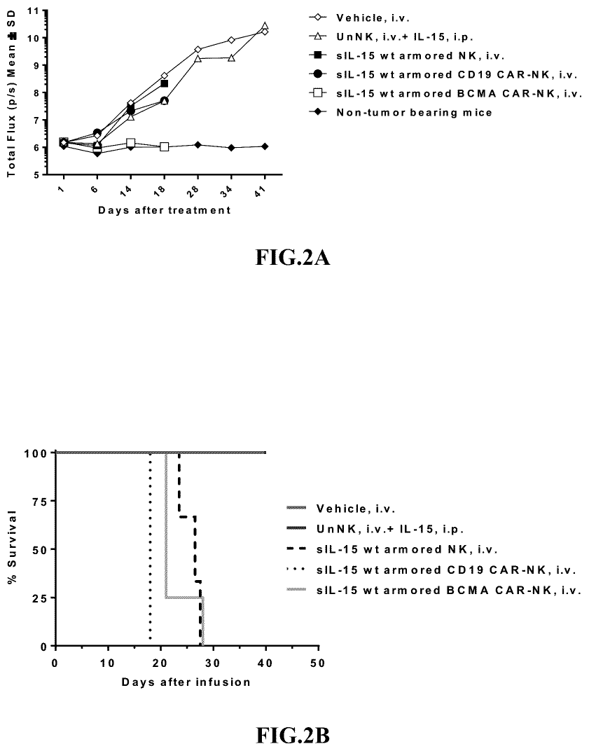

Mice were infused with 2 M CAR⁺ NK cells at day 0, 2, and day 5 respectively. Tumor progression was evaluated by in vivo bioluminescence imaging (BLI) weekly at each time point. As shown in FIGS. 2A-2B, sIL-15 wt armored NK cells, sIL-15 wt armored CD19 CAR-NK and sIL-15 wt armored BCMA CAR-NK cells showed similar toxicity in mice (dying between day 18 and day 28). Thus, the observed toxicity is from the wildtype IL-15 armor.

### Example 4: In Vivo Evaluation of Mutant IL-15 Armored BCMA CAR-NK Cells

This example shows that mutant IL-15 armored BCMA CAR-NK cells demonstrate strong anti-tumor effects without inducing uncontrolled cytokine release (e.g., cytokine storm).

BCMA CAR constructs and tumor xenograft mice were prepared as described in Examples 1 and 3. Tumor engrafted NCG mice were treated with sIL-15 wt armored BCMA CAR-NK cells, and sIL-15 m1 to m8 armored BCMA CAR-NK cells. Mice were infused with 2 M CAR+NK cells at day 0, 2, and day 5 respectively. Tumor progression was evaluated by in vivo bioluminescence imaging (BLI).

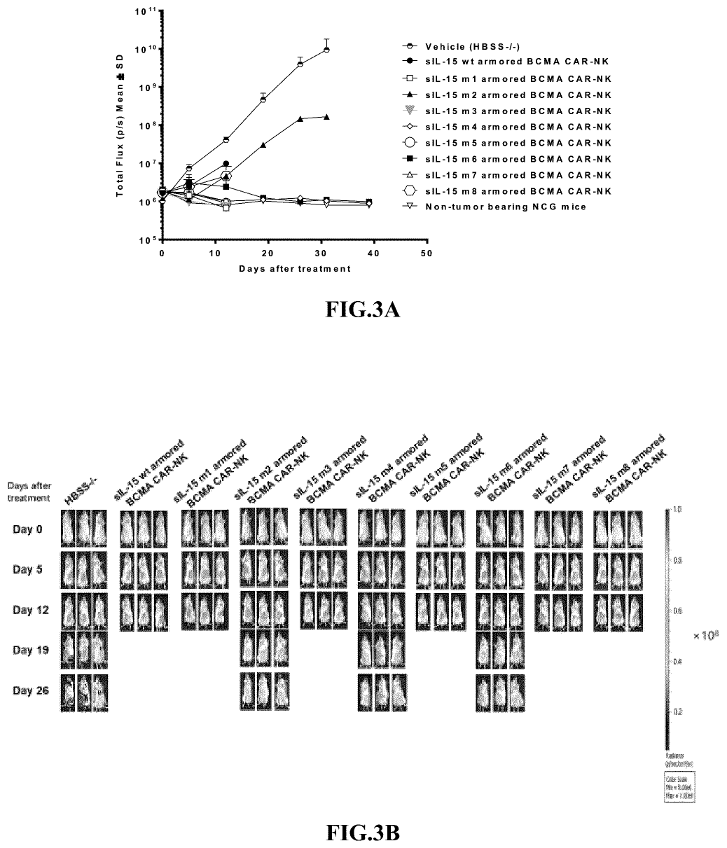

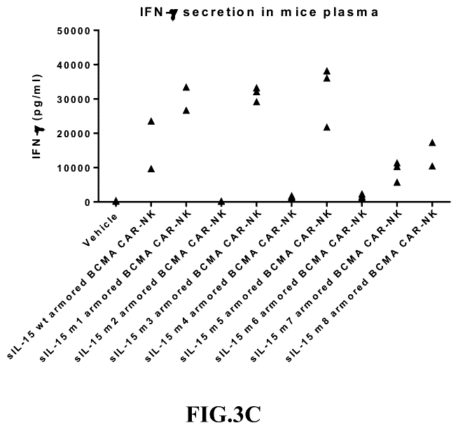

As shown in FIGS. 3A-3B, sIL-15 m1, m3, m4, m5, m6, and m7 armored BCMA CAR-NK cells showed potent anti-tumor efficacy. All mice treated with m4 and m6 mutant IL-15 armored BCMA CAR-NK cells survived the treatment, whereas mice treated with m1, m3, m5, m7, and m8 mutant IL-15 armored BCMA CAR-NK cells showed toxicity, with mice dying between day 10 to day 20. Mice in the groups of sIL-15 m2, m4 and m6 armored BCMA CAR-NK cells survived longer than others did, of which IFN-γ release were low (FIG. 3C), indicating that the cytokine release level is correlates with toxicity in mice. The IFN-γ secretion levels in plasma of each mouse shown in FIG. 3C are quantified in Table 6.

TABLE 6
IFN-γ secretion levels in mice plasma (pg/mL)

| Treatment | Mouse | Mouse | Mouse |
|---|---|---|---|
| group | No. 1 | No. 2 | No. 3 |
| Vehicle (HBSS−/−) | 428.88 | 195.69 | 279.66 |
| sIL-15 wt armored BCMA CAR-NK | 9731.12 | 23599.15 | — |
| sIL-15 m1 armored BCMA CAR-NK | 33490.79 | 26717.84 | 26641.33 |
| sIL-15 m2 armored BCMA CAR-NK | 313.06 | 183.15 | 198.76 |
| sIL-15 m3 armored BCMA CAR-NK | 33257.51 | 32146.80 | 29236.07 |
| sIL-15 m4 armored BCMA CAR-NK | 1828.57 | 1317.82 | 1559.97 |
| sIL-15 m5 armored BCMA CAR-NK | 21818.91 | 38167.39 | 36124.08 |
| sIL-15 m6 armored BCMA CAR-NK | 2379.98 | 1639.06 | 1290.88 |
| sIL-15 m7 armored BCMA CAR-NK | 10350.81 | 5773.59 | 11396.86 |
| sIL-15 m8 armored BCMA CAR-NK | 10486.21 | 17361.44 | — |

### Example 5: In Vivo Evaluation of IL-15 m6 Armored CAR-NK Cells

This example shows that secreted IL-15 m6 mutant armored BCMA CAR-NK cells demonstrate strong anti-tumor effects without inducing uncontrolled cytokine release (e.g., cytokine storm).

BCMA CAR constructs and tumor xenograft mice were prepared as described in Examples 1 and 3. After fourteen days, tumor engrafted NCG mice (high tumor burden) were treated with membrane bound wildtype IL-15 (i.e., “mbIL-15 wt”) armored BCMA CAR-NK cells, sIL-15 wt armored BCMA CAR-NK cells, and sIL-15 m6 armored BCMA CAR-NK cells. Mice were infused with 4.5 M CAR⁺ NK cells at day 0, 2, and day 5 respectively. Tumor progression was evaluated by in vivo bioluminescence imaging (BLI).

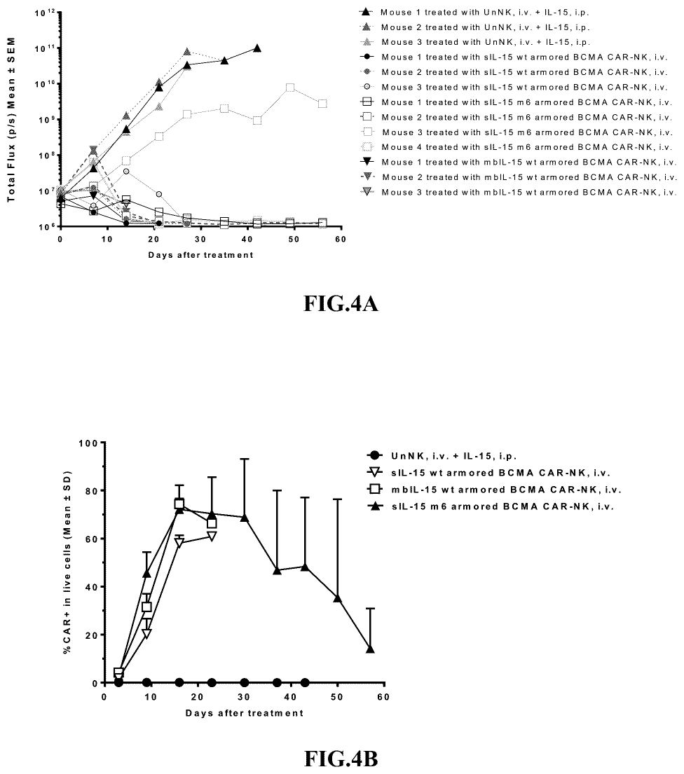

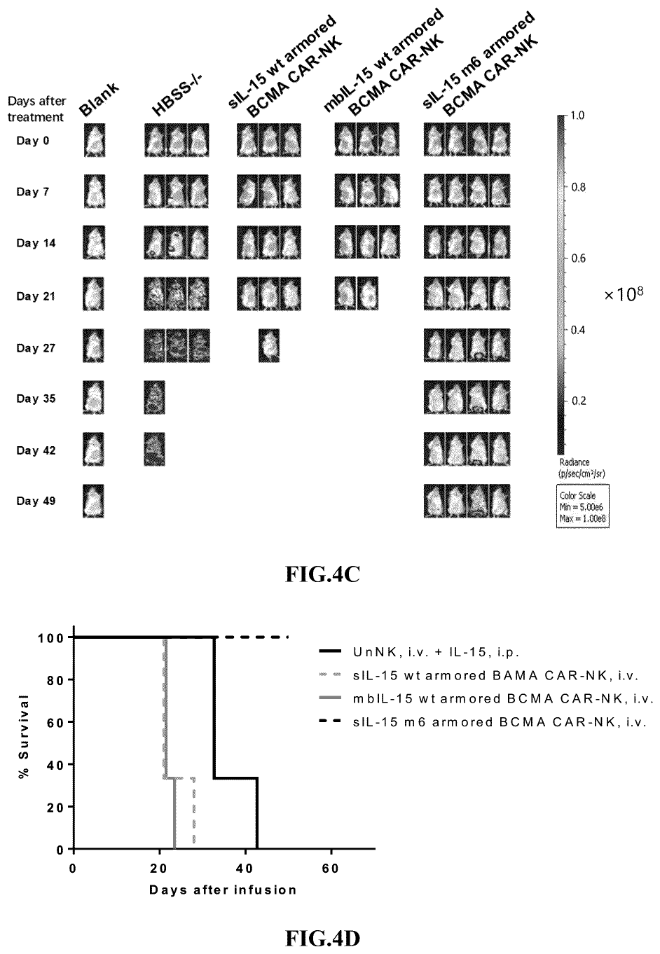

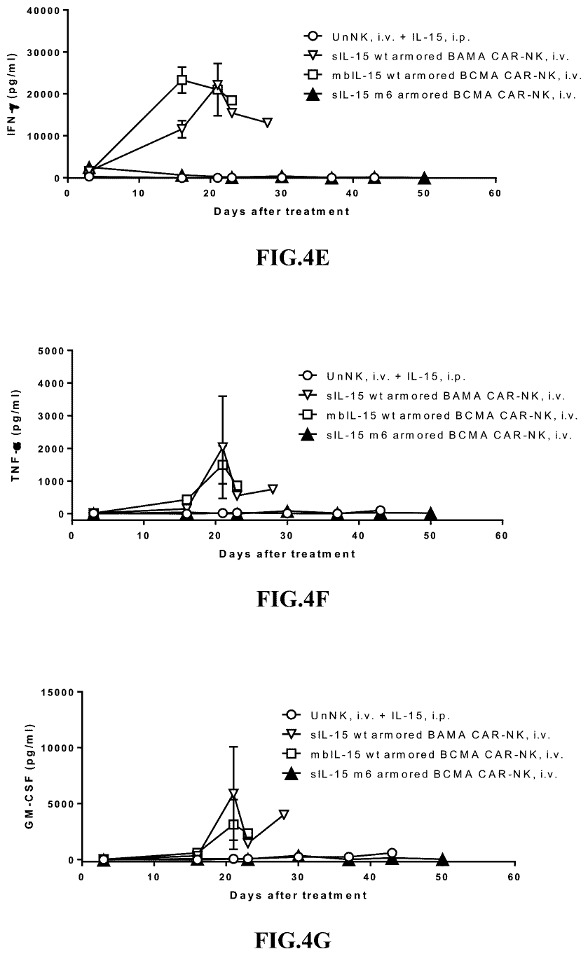

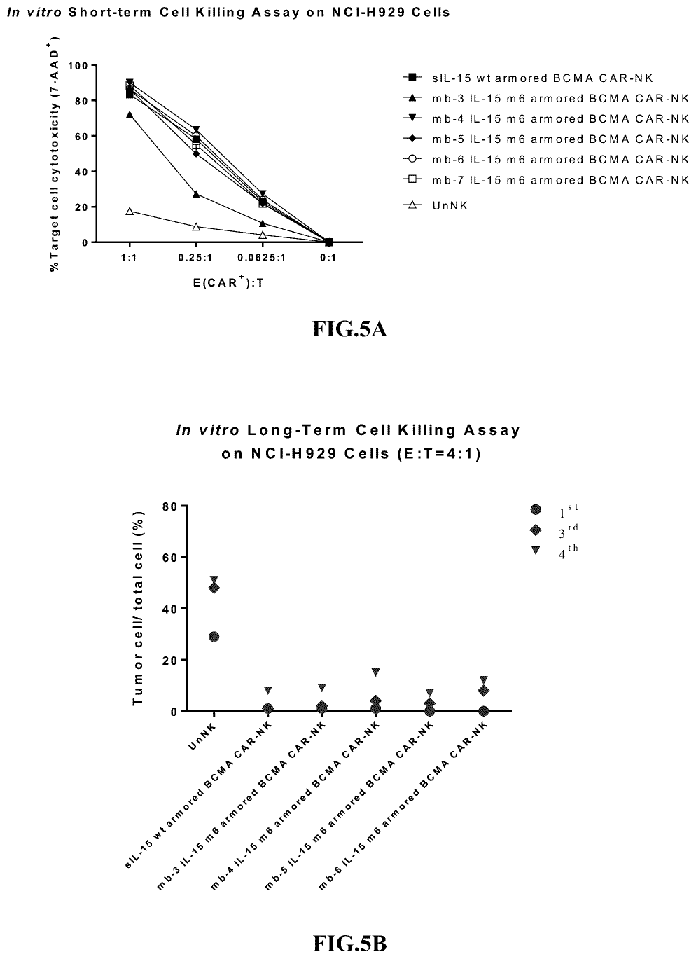

FIGS. 4A-4G show that mice treated with sIL-15 wt armored BCMA CAR-NK cells and mbIL-15 wt armored BCMA CAR-NK cells died between day 21 to day 27, indicating that these constructs are cytotoxic to the mice. The high pro-inflammatory cytokine release (e.g., IFN-γ, TNF-α, and GM-CSF) of mice treated with sIL-15 wt armored BCMA CAR-NK cells and mbIL-15 wt armored BCMA CAR-NK cells correlates with the observed toxicity (FIGS. 4E-4G). Conversely, low level of pro-inflammatory cytokine release in mice treated with mutant sIL-15 m6 armored BCMA CAR-NK cells correlates with a higher survival rate (FIG. 4D). Three of the four mice in the group treated with mutated sIL-15 m6 armored BCMA CAR-NK cells showed potent anti-tumor efficacy, and all the mice in the group survived the treatment. One mouse in the group of mutant sIL-15 m6 armored BCMA CAR-NK cells maintained the tumor burden even after treatment. By the analysis of the BCMA antigen in the tumor cells from this mice, it was determined that there was a loss of BCMA antigen in these tumor cells (data not shown).

Overall, m4 and m6 mutant IL-15 armored BCMA CAR-NK cells have improved anti-tumor efficacy and lower toxicity compared to BCMA CAR-NK cells bearing IL-15 wt or other IL-15 mutated constructs in mouse models.

### Example 6: Preparation and Evaluation of Membrane Bound Mutant IL-15 Armored CAR-NK Cells

NK cells expressing various constructs of soluble or membrane-bound IL-15 armored CAR were prepared. Table 7 below describes the structures of the constructs. The anti-tumor efficacy and toxicity of these IL-15 armored CAR-NK cells were evaluated in vivo using the mice model described in Example 3. Mice were infused with one dose of 1 M CAR+NK cells. Similar constructs can be made based on other IL-15 muteins and CARs described herein.

This example shows that membrane bound mutant IL-15 armored BCMA CAR-NK cells demonstrate strong anti-tumor effects without inducing uncontrolled cytokine release (e.g., cytokine storm).

TABLE 7
Exemplary Constructs of mutant IL-15 armored CAR

| CAR Name | IL-15 form | Expression Structure |
|---|---|---|
| sIL-15 m6-Rα armored | sIL-15 m6-Rα | SP-IL15 m6-IL15Rα Extracellular Domain (ED)- |
| BCMA CAR | | P2A-CD8αSP-VHH1-VHH2-CD8αTM-4-1BB - |
| | | CD3zeta |
| mb-1 IL-15 m6 armored | mb-1 IL-15 m6 | SP-IL15 m6-IL15Rα Suchi Domain-P2A- |
| BCMA CAR | | CD8αSP-VHH1-VHH2-CD8αTM-4-1BB - |
| | | CD3zeta |
| mb-2 IL-15 m6 armored | mb-2 IL-15 m6 | SP-IL15 m6-linker - VHH1-VHH2-CD8αTM-4- |
| BCMA CAR | | 1BB -CD3zeta |
| mb-3 IL-15 m6 armored | mb-3 IL-15 m6 | CD8αSP-VHH1-VHH2-linker- IL15 m6-linker- |
| BCMA CAR | | CD8αTM-4-1BB -CD3zeta |
| mb-4 IL-15 m6 armored | mb-4 IL-15 m6 | SP-IL15 m6-linker-IL15Rβ-P2A-γc -P2A- |
| BCMA CAR | | CD8αSP-VHH1-VHH2-CD8αTM-4-1BB - |
| | | CD3zeta |
| mb-5 IL-15 m6 armored | mb-5 IL-15 m6 | SP-IL15 m6-linker-IL15Rα-P2A-CD8αSP- |
| BCMA CAR | | VHH1-VHH2-CD8αTM-4-1BB -CD3zeta |
| mb-6 IL-15 m6 armored | mb-6 IL-15 m6 | SP-IL15 m6-linker-IL15Rβ-P2A-CD8αSP- |
| BCMA CAR | | VHH1-VHH2-CD8αTM-4-1BB -CD3zeta |
| mb-7 IL-15 m6 armored | mb-7 IL-15 m6 | SP-IL15 m6-linker-γc-P2A-CD8αSP-VHH1- |
| BCMA CAR | | VHH2-CD8αTM-4-1BB -CD3zeta |
| mb-8 IL-15 m6 armored | mb-8 IL-15 m6 | SP-IL15 m6-linker-IL15RαTM-P2A-CD8αSP- |
| BCMA CAR | | VHH1-VHH2-CD8αTM-4-1BB -CD3zeta |
| mb-9 IL-15 m4 armored | mb-9 IL-15 m4 | CD8αSP-VHH1-VHH2-linker-m4-linker- |
| BCMA CAR | | CD8αTM-4-1BB -CD3zeta |
| mb-10 IL-15 m4 | mb-10 IL-15 m4 | SP-m4- linker -IL-15Rβ-P2A-γc -P2A-CD8αSP- |
| armored BCMA CAR | | VHH1-VHH2-CD8αTM-4-1Bβ -CD3zeta |
| mb-11 IL-15 m4 | mb-11 IL-15 m4 | SP-m4 - linker -IL-15Rα-P2A-CD8αSP-VHH1- |
| armored BCMA CAR | | VHH2-CD8αTM-4-1BB -CD3zeta |

The amino acid sequence of mb-3 IL-15 m6 armored BCMA CAR described above is shown in SEQ ID NO: 66. To achieve membrane binding of IL-15 m6, other exemplary expression structures of membrane bound IL-15 m6 were also constructed, including mb-4 IL-15 m6 (SEQ ID NO: 60), mb-5 IL-15 m6 (SEQ ID NO: 61), mb-6 IL-15 m6 (SEQ ID NO: 62), and mb-7 IL-15 m6 (SEQ ID NO: 63). Membrane bound IL-15 m6 armored BCMA CAR was constructed by fusing membrane bound IL-15 m6 and BCMA CAR through P2A, as shown in Table 7.

The amino acid sequence of mb-9 IL-15 m4 armored BCMA CAR described above is shown in SEQ ID NO: 75. To achieve membrane binding of IL-15 m4, other exemplary expression structures of membrane bound IL-15 m4 were also constructed, including mb-10 IL-15 m4 (SEQ ID NO: 76) armored BCMA CAR and mb-11 IL-15 m4 (SEQ ID NO: 77) armored BCMA CAR. Membrane bound IL-15 m4 armored BCMA CAR was constructed by fusing membrane bound IL-15 m4 and BCMA CAR through P2A, as shown in Table 7.

The membrane bound IL-15 m6 armored CAR-NK cells were tested in vitro for cytotoxicity in a short-term (FIG. 5A) and long-term (FIG. 5B) cell killing assay. FIG. 5A shows in vitro short-term (4 hours) killing of NCI-H929 cells by the membrane bound IL-15 m6 armored BCMA CAR-NK cells (E:T=1:1, 0.25:1, 0.0625:1). With different m6-bearing IL-15 membrane-bound structures, mb-3, mb-4, mb-5, mb-6 and mb-7 IL-15 m6 armored BCMA CAR-NK cells showed potent anti-tumor efficacy in the short-term killing assay compared to UnNK controls. The percentage of cytotoxicity on target cells was calculated by 7-AAD+%: 7-AAD+cells/Target cells×100%.

FIG. 5B shows the in vitro long-term killing of the membrane bound mutant IL-15 m6 (e.g., mb-3, mb-4, mb-5 and mb-6) armored BCMA CAR-NK cells on NCI-H929 cells (E:T=1:4). In each run of stimulation, tumor cells were added into NK cells for co-culture about 24 hours to 48 hours, and after co-culture, cells were collected for further analyzed by flow cytometry. Runs were repeated for a total of 4 antigen stimulations. As shown in FIG. 5B, the membrane bound mutant IL-15 m6 (e.g., mb-3, mb-4, mb-5, and mb-6) armored BCMA CAR-NK cells showed similar anti-tumor efficacy compared to sIL-15 wt armored BCMA CAR-NK cells.

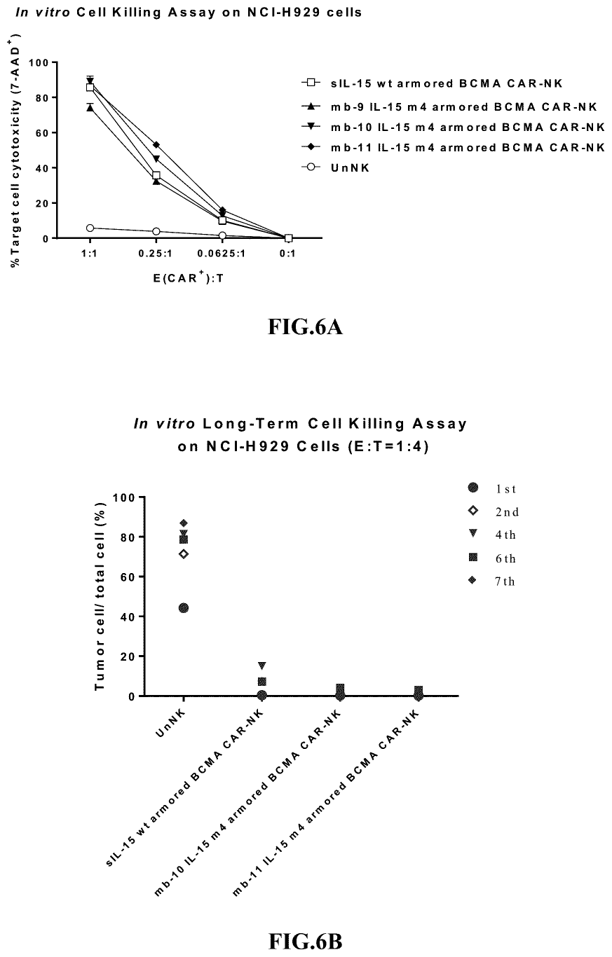

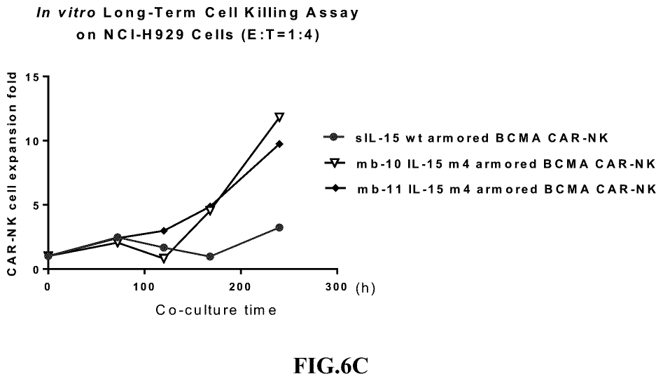

The membrane bound IL-15 m4 armored CAR-NK cells were also tested in vitro for cytotoxicity in a short-term (FIG. 6A) and long-term (FIG. 6B and FIG. 6C) cell killing assay. FIG. 6A shows in vitro short-term (4 hours) killing of NCI-H929 cells by the membrane bound IL-15 m4 (mb-9, mb-10 and mb-11) armored BCMA CAR-NK cells (E:T=1:1, 0.25:1, 0.0625:1). With different m4-bearing IL-15 membrane-bound structures, mb-9, mb-10 and mb-11 IL-15 m4 armored BCMA CAR-NK cells showed potent anti-tumor efficacy in the short-term killing assay compared to UnNK controls. The percentage of cytotoxicity on target cells was calculated by 7-AAD+%: 7-AAD+cells/Target cells×100%.

FIG. 6B shows the in vitro long-term killing of the membrane bound mutant IL-15 m4 (e.g., mb-10 and mb-11) armored BCMA CAR-NK cells on NCI-H929 cells (E:T=1:4). In each run of stimulation, tumor cells were added into NK cells for co-culture about 24 hours to 48 hours, and after co-culture, cells were collected for further analyzed by flow cytometry. Runs were repeated for a total of 7 antigen stimulations. As shown in FIG. 6B, the membrane bound mutant IL-15 m4 (e.g., mb-10 and mb-11) armored BCMA CAR-NK cells showed better anti-tumor efficacy compared to sIL-15 wt armored BCMA CAR-NK cells. During the stimulation with tumor cells, mb-10 IL-15 m4 armored BCMA CAR-NK cells and mb-11 IL-15 m4 armored BCMA CAR-NK cells additionally showed better expansion (FIG. 6C).

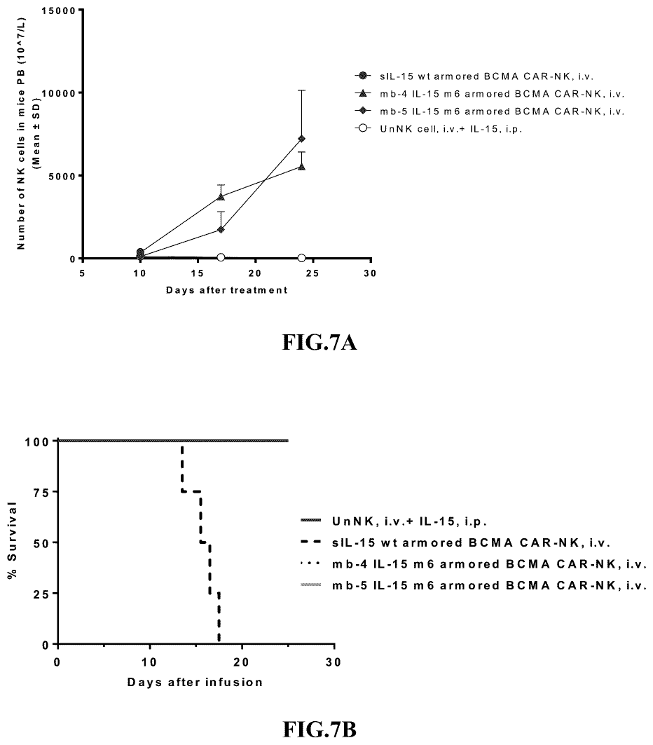

FIGS. 7A-7B show in vivo evaluation of BCMA CAR-NK cells armored with sIL-15 wt and membrane bound IL-15 m6 against BCMA-positive target cells, NCI-H929, in a NCG mouse model (NCI-H929-Luc model) as described above. FIG. 7A shows BCMA CAR PK in mouse peripheral blood. The membrane bound mutant IL-15 m6 (mb-4 and mb-5) armored BCMA CAR-NK cells showed good expansion in mouse peripheral blood. FIG. 7B shows the survival curve of mice treated with sIL-15 wt armored BCMA CAR-NK cells and membrane bound mutated IL-15 (mb-4 IL-15 m6 and mb-5 IL-15 m6) armored BCMA CAR-NK cells. The mice in group of sIL-15 wt armored BCMA CAR-NK cells died between among day 13 to day 17 post CAR-NK cell infusion. Mice in groups of membrane bound mutated IL-15 (mb-4 IL-15 m6 and mb-5 IL-15 m6) armored BCMA CAR-NK cells survived longer.

### Example 7: Assays for the Evaluation of In Vitro Activities of Mutant IL-15 Armored GPC3 CAR-NK Cells

### Generation of Mutant IL-15 Armored GPC3 CAR

To prepare the GPC3 CAR, the scfv of an anti-GPC3 antibody (SEQ ID NO: 80) was fused with a CAR backbone comprising from the N-terminus to the C-terminus: a CD8α hinge domain, a CD8α transmembrane domain, a 4-1BB co-stimulatory domain and a CD3ζ intracellular domain (SEQ ID NO: 85). The CAR backbone was operably linked to a CD8α signal peptide fused to the N-terminus of GPC3 scFv. The amino acid sequence of the GPC3 CAR is shown in SEQ ID NO: 81. Wild type IL-15 or IL-15 mutein (m17 or m18) was fused with the GPC3 CAR using a 2A self-cleaving peptide linker, hereinafter referred to as “sIL-15 wt armored GPC3 CAR”, “sIL-15 m17 armored GPC3 CAR” and “sIL-15 m18 armored GPC3 CAR”, also see Table 5. The nucleic acids encoding the polypeptides were cloned into a retroviral vector as described above. Mutant IL-15 armored GPC3 CAR-NK cells were generated using the methods described in Example 1.

### Cell Cytotoxicity Assay-Luciferase Assay

For the short-term cell cytotoxicity assay, mutant IL-15 armored GPC3 CAR-NK cells were co-cultured with luciferase-expressing Huh7 cells (GPC3 positive, Huh7/Luc) at 1:10 of effector to target ratios (E:T) at 37° C. for 72 hours. The residual luciferase activity (Lucᵣₑₛᵢ) was determined by ONE-Glo luciferase assay system (Promega) following the users' manual. The same number of target cells cultured without effector cells was used as a control (Lucₘₐₓ). The percentage of target cell lysis was calculated as following formulation: (Lucₘₐₓ-Lucᵣₑₛᵢ)/Lucₘₐₓ×100%.

For the long-term cell cytotoxicity assay, mutant IL-15 armored GPC3 CAR-NK cells were co-cultured with Huh7/Luc cells (E/T=1:2) at 37° C. in 6-well plate. Every 24 hours (as a round of stimulation), 10% of the cell mixture was collected and subjected to flow cytometry assay to determine the percentage of residual target cells, while the rest of the cells was co-incubated with the newly added 4×10⁵Huh7/Luc cells.

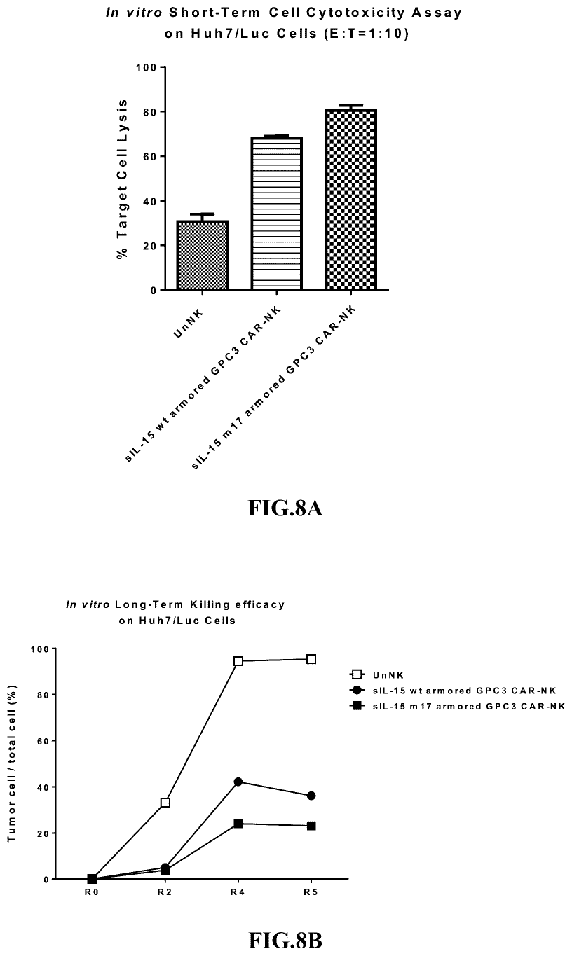

sIL-15 m17 armored GPC3 CAR-NK cells were tested in vitro for cytotoxicity in a short-term (FIG. 8A) and long-term (FIG. 8B) cell killing assay. As shown in FIG. 8A, at the E:T ratio of 1:10, sIL-15 m17 armored GPC3 CAR-NK cells with higher target cell lysis percentage showed potent anti-tumor efficacy against Huh7 cells in the short-term (72 hours) killing assay compared to sIL-15 wt armored GPC3 CAR-NK cells. The same results were also observed in a long-term cell killing assay after the second round (R2) of stimulation (FIG. 8B).

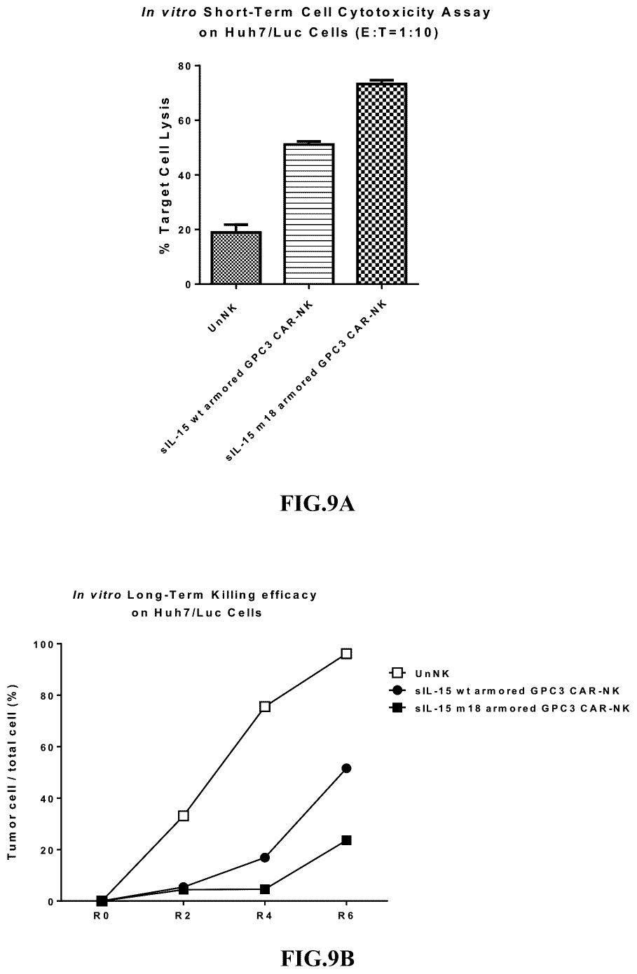

sIL-15 m18 armored GPC3 CAR-NK cells were tested in vitro for cytotoxicity in a short-term (FIG. 9A) and long-term (FIG. 9B) cell killing assay. As shown in FIG. 9A, at the E:T ratio of 1:10, sIL-15 m18 armored GPC3 CAR-NK cells with higher target cell lysis percentage showed potent anti-tumor efficacy against Huh7 cells in the short-term (72 hours) killing assay compared to sIL-15 wt armored GPC3 CAR-NK cells. The same results were also observed in a long-term cell killing assay after the second round (R2) of stimulation (FIG. 9B).

SEQUENCE LISTING

SEQ ID NO: 1. wild type hIL-15
NWVNVISDLKKIEDLIQSMHIDATLYTESDVHPSCKVTAMKCFLLELQVISLESGDASIHDT
VENLIILANNSLSSNGNVTESGCKECEELEEKNIKEFLQSFVHIVQMFINTS
SEQ ID NO: 2. IL15 mutein 1
NWVNVISDLKKIEDLIQSMHIDLTLYTESDVHPSCKVTAMKCFLLELQVISLESGDASIHDT
VENLIILANNSLSSNGNVTESGCKECEELEEKNIKEFLQSFVHIVQMFINTS
SEQ ID NO: 3. IL15 mutein 2
NWVNVISDLKKIEDLIQSMHIDATEYTESDVHPSCKVTAMKCFLLELQVISLESGDASIHDT
VENLIILANNSLSSNGNVTESGCKECEELEEKNIKEFLQSFVHIVQMFINTS
SEQ ID NO: 4. IL15 mutein 3
NWVNVISDLKKIEDLIQSMHIDATLGTESDVHPSCKVTAMKCFLLELQVISLESGDASIHDT
VENLIILANNSLSSNGNVTESGCKECEELEEKNIKEFLQSFVHIVQMFINTS
SEQ ID NO: 5. IL15 mutein 4
NWVNVISELKKIEDLIQSMHIDATLYTESDVHPSCKVTAMKCFLLELQVISLESGDASIHDT
VENLIILANNSLSSNGNVTESGCKECEELEEKNIKEFLQSFVHIVQMFINTS
SEQ ID NO: 6. IL15 mutein 5
NWVNVISDLKKIEDLIQSMHIDATLYTESDVHPSCKVTAMKCFLLELQVISLESGDASIHET
VENLIILANNSLSSNGNVTESGCKECEELEEKNIKEFLQSFVHIVQMFINTS
SEQ ID NO: 7. IL15 mutein 6
NWVNVISDLKKIEDLIQSMHIDATLYTESDVHPSCKVTAMKCFLLELQVISLESGDASIHDG
VENLIILANNSLSSNGNVTESGCKECEELEEKNIKEFLQSFVHIVQMFINTS
SEQ ID NO: 8. IL15 mutein 7
NWVNVISDLKKIEDLIQSMHIDATLYTESDVHPSCKVTAMKCFLLELQVISLESGDASIHDIV
ENLIILANNSLSSNGNVTESGCKECEELEEKNIKEFLQSFVHIVQMFINTS
SEQ ID NO: 9. IL15 mutein 8
NWVNVISDLKKIEDLIQSMHIDATLYTESDVHPSCKVTAMKCFLLELQVISLESGDASIHDT
VENLIILANNSLSSNGNVTESGCKECKELEEKNIKEFLQSFVHIVQMFINTS
SEQ ID NO: 10. IL15 mutein 9
NWVNVISDLKKIEDLIQSMHIDATLYTESDVHPSCKVTAMKCFLLELQVISLESGDADIHDT
VENLIILANNSLSSNGNVTESGCKECEELEEKNIKEFLQSFVHIVQMFINTS
SEQ ID NO: 11. IL15 mutein 10
NWVNVISDLKKIEDLIQSMHIDATLYTESDVHPSCKVTAMKCFLLELQVISLESGDASIHDQ
VENLIILANNSLSSNGNVTESGCKECEELEEKNIKEFLQSFVHIVQMFINTS
SEQ ID NO: 12. IL15 mutein 11
NWVNVISDLKKIEDLIQSMHIDATLYTESDVHPSCKVTAMKCFLLELQVISLESGDASIHDV
VENLIILANNSLSSNGNVTESGCKECEELEEKNIKEFLQSFVHIVQMFINTS
SEQ ID NO: 13. IL15 mutein 12
NWVNVISDLKKIEDLIQSMHIDATLYTESDVHPSCKVTAMKCFLLELQVISLESGDASIHDP
VENLIILANNSLSSNGNVTESGCKECEELEEKNIKEFLQSFVHIVQMFINTS
SEQ ID NO: 14. IL15 mutein 13
NWVNVISDLKKIEDLIQSMHIDATLYTESDVHPSCKVTAMKCFLLELQVISLESGDASIHDL
VENLIILANNSLSSNGNVTESGCKECEELEEKNIKEFLQSFVHIVQMFINTS
SEQ ID NO: 15. IL15 mutein 14
NWVNVISDLKKIEDLIQSMHIDATLYTESDVHPSCKVTAMKCFLLELQVISLESGDASIHDA
VENLIILANNSLSSNGNVTESGCKECEELEEKNIKEFLQSFVHIVQMFINTS
SEQ ID NO: 16. IL15 mutein 15
NWVNVISDLKKIEDLIQSMHIDATLYTESDVHPSCKVTAMKCFLLELQVISLESGDASIHDS
VENLIILANNSLSSNGNVTESGCKECEELEEKNIKEFLQSFVHIVQMFINTS
SEQ ID NO: 17. IL15 mutein 16
NWVNVISDLKKIEDLIQSMHIDATLYTESDVHPSCKVTAMKCFLLELQVISLESGDASIHDY
VENLIILANNSLSSNGNVTESGCKECEELEEKNIKEFLQSFVHIVQMFINTS
SEQ ID NO: 18. CD8α hinge
TTTPAPRPPTPAPTIASQPLSLRPEACRPAAGGAVHTRGLDFACD
SEQ ID NO: 19. CD8α TM
IYIWAPLAGTCGVLLLSLVITLYC
SEQ ID NO: 20. 4-1BB co-stimulatory signaling domain
KRGRKKLLYIFKQPFMRPVQTTQEEDGCSCRFPEEEEGGCEL
SEQ ID NO: 21. CD3ζ primary intracellular signaling domain
RVKFSRSADAPAYQQGQNQLYNELNLGRREEYDVLDKRRGRDPEMGGKPRRKNPQEGLY
NELQKDKMAEAYSEIGMKGERRRGKGHDGLYQGLSTATKDTYDALHMQALPPR
SEQ ID NO: 22. CD8αSP
MALPVTALLLPLALLLHAARP
SEQ ID NO: 23. BCMA VHH1-VHH2
AVQLVESGGGLVQAGDSLRLTCTASGRAFSTYFMAWFRQAPGKEREFVAGIAWSGGSTAY
ADSVKGRFTISRDNAKNTVYLQMNSLKSEDTAVYYCASRGIEVEEFGAWGQGTQVTVSSG
GGGSQVQLEESGGGSVQAGGSLRLSCAYTYSTYSNYYMGWFREAPGKARTSVAIISSDTTI
TYKDAVKGRFTISKDNAKNTLYLQMNSLKPEDSAMYRCAAWTSDWSVAYWGQGTQVTV
SS
SEQ ID NO: 24. CD19 scFv
DIQMTQTTSSLSASLGDRVTISCRASQDISKYLNWYQQKPDGTVKLLIYHTSRLHSGVPSRF
SGSGSGTDYSLTISNLEQEDIATYFCQQGNTLPYTFGGGTKLEITGGGGSGGGGSGGGGSEV
KLQESGPGLVAPSQSLSVTCTVSGVSLPDYGVSWIRQPPRKGLEWLGVIWGSETTYYNSAL
KSRLTIIKDNSKSQVFLKMNSLQTDDTAIYYCAKHYYYGGSYAMDYWGQGTSVTVSS
SEQ ID NO: 25. hIL15-IL15RaTM
NWVNVISDLKKIEDLIQSMHIDATLYTESDVHPSCKVTAMKCFLLELQVISLESGDASIHDT
VENLIILANNSLSSNGNVTESGCKECEELEEKNIKEFLQSFVHIVQMFINTSGGGGSGGGGSG
GGGSVAISTSTVLLCGLSAVSLLACYL
SEQ ID NO: 26. BCMA CAR
MALPVTALLLPLALLLHAARPAVQLVESGGGLVQAGDSLRLTCTASGRAFSTYFMAWFRQ
APGKEREFVAGIAWSGGSTAYADSVKGRFTISRDNAKNTVYLQMNSLKSEDTAVYYCASR
GIEVEEFGAWGQGTQVTVSSGGGGSQVQLEESGGGSVQAGGSLRLSCAYTYSTYSNYYMG
WFREAPGKARTSVAIISSDTTITYKDAVKGRFTISKDNAKNTLYLQMNSLKPEDSAMYRCA
AWTSDWSVAYWGQGTQVTVSSTSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQ
ALPPR
SEQ ID NO: 27. CD19 CAR
MALPVTALLLPLALLLHAARPDIQMTQTTSSLSASLGDRVTISCRASQDISKYLNWYQQKP
DGTVKLLIYHTSRLHSGVPSRFSGSGSGTDYSLTISNLEQEDIATYFCQQGNTLPYTFGGGTK
LEITGGGGSGGGGSGGGGSEVKLQESGPGLVAPSQSLSVTCTVSGVSLPDYGVSWIRQPPR
KGLEWLGVIWGSETTYYNSALKSRLTIIKDNSKSQVFLKMNSLQTDDTAIYYCAKHYYYG
GSYAMDYWGQGTSVTVSSTSTTTPAPRPPTPAPTIASQPLSLRPEACRPAAGGAVHTRGLDF
ACDIYIWAPLAGTCGVLLLSLVITLYCKRGRKKLLYIFKQPFMRPVQTTQEEDGCSCRFPEE
EEGGCELRVKFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYDALHMQALPP
R
SEQ ID NO: 28. P2A
GSGATNFSLLKQAGDVEENPGP
SEQ ID NO: 29. sIL-15 wt armored BCMA CAR
MALPVTALLLPLALLLHAARPAVQLVESGGGLVQAGDSLRLTCTASGRAFSTYFMAWFRQ
APGKEREFVAGIAWSGGSTAYADSVKGRFTISRDNAKNTVYLQMNSLKSEDTAVYYCASR
GIEVEEFGAWGQGTQVTVSSGGGGSQVQLEESGGGSVQAGGSLRLSCAYTYSTYSNYYMG
WFREAPGKARTSVAIISSDTTITYKDAVKGRFTISKDNAKNTLYLQMNSLKPEDSAMYRCA
AWTSDWSVAYWGQGTQVTVSSTSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQ
ALPPRGSGATNFSLLKQAGDVEENPGPMRISKPHLRSISIQCYLCLLLNSHFLTEAGIHVFILG
CFSAGLPKTEANWVNVISDLKKIEDLIQSMHIDATLYTESDVHPSCKVTAMKCFLLELQVIS
LESGDASIHDTVENLIILANNSLSSNGNVTESGCKECEELEEKNIKEFLQSFVHIVQMFINTS
SEQ ID NO: 30. sIL-15 wt armored CD19 CAR
MALPVTALLLPLALLLHAARPDIQMTQTTSSLSASLGDRVTISCRASQDISKYLNWYQQKP
DGTVKLLIYHTSRLHSGVPSRFSGSGSGTDYSLTISNLEQEDIATYFCQQGNTLPYTFGGGTK
LEITGGGGSGGGGSGGGGSEVKLQESGPGLVAPSQSLSVTCTVSGVSLPDYGVSWIRQPPR
KGLEWLGVIWGSETTYYNSALKSRLTIIKDNSKSQVFLKMNSLQTDDTAIYYCAKHYYYG
GSYAMDYWGQGTSVTVSSTSTTTPAPRPPTPAPTIASQPLSLRPEACRPAAGGAVHTRGLDF
ACDIYIWAPLAGTCGVLLLSLVITLYCKRGRKKLLYIFKQPFMRPVQTTQEEDGCSCRFPEE
EEGGCELRVKFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYDALHMQALPP
RGSGATNFSLLKQAGDVEENPGPMRISKPHLRSISIQCYLCLLLNSHFLTEAGIHVFILGCFSA
GLPKTEANWVNVISDLKKIEDLIQSMHIDATLYTESDVHPSCKVTAMKCFLLELQVISLESG
DASIHDTVENLIILANNSLSSNGNVTESGCKECEELEEKNIKEFLQSFVHIVQMFINTS
SEQ ID NO: 31. sIL-15 m1 armored BCMA CAR
MALPVTALLLPLALLLHAARPAVQLVESGGGLVQAGDSLRLTCTASGRAFSTYFMAWFRQ
APGKEREFVAGIAWSGGSTAYADSVKGRFTISRDNAKNTVYLQMNSLKSEDTAVYYCASR
GIEVEEFGAWGQGTQVTVSSGGGGSQVQLEESGGGSVQAGGSLRLSCAYTYSTYSNYYMG
WFREAPGKARTSVAIISSDTTITYKDAVKGRFTISKDNAKNTLYLQMNSLKPEDSAMYRCA
AWTSDWSVAYWGQGTQVTVSSTSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQ
ALPPRGSGATNFSLLKQAGDVEENPGPMRISKPHLRSISIQCYLCLLLNSHFLTEAGIHVFILG
CFSAGLPKTEANWVNVISDLKKIEDLIQSMHIDLTLYTESDVHPSCKVTAMKCFLLELQVIS
LESGDASIHDTVENLIILANNSLSSNGNVTESGCKECEELEEKNIKEFLQSFVHIVQMFINTS
SEQ ID NO: 32. sIL-15 m2 armored BCMA CAR
MALPVTALLLPLALLLHAARPAVQLVESGGGLVQAGDSLRLTCTASGRAFSTYFMAWFRQ
APGKEREFVAGIAWSGGSTAYADSVKGRFTISRDNAKNTVYLQMNSLKSEDTAVYYCASR
GIEVEEFGAWGQGTQVTVSSGGGGSQVQLEESGGGSVQAGGSLRLSCAYTYSTYSNYYMG
WFREAPGKARTSVAIISSDTTITYKDAVKGRFTISKDNAKNTLYLQMNSLKPEDSAMYRCA
AWTSDWSVAYWGQGTQVTVSSTSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQ
ALPPRGSGATNFSLLKQAGDVEENPGPMRISKPHLRSISIQCYLCLLLNSHFLTEAGIHVFILG
CFSAGLPKTEANWVNVISDLKKIEDLIQSMHIDATEYTESDVHPSCKVTAMKCFLLELQVIS
LESGDASIHDTVENLIILANNSLSSNGNVTESGCKECEELEEKNIKEFLQSFVHIVQMFINTS
SEQ ID NO: 33. sIL-15 m3 armored BCMA CAR
MALPVTALLLPLALLLHAARPAVQLVESGGGLVQAGDSLRLTCTASGRAFSTYFMAWFRQ
APGKEREFVAGIAWSGGSTAYADSVKGRFTISRDNAKNTVYLQMNSLKSEDTAVYYCASR
GIEVEEFGAWGQGTQVTVSSGGGGSQVQLEESGGGSVQAGGSLRLSCAYTYSTYSNYYMG
WFREAPGKARTSVAIISSDTTITYKDAVKGRFTISKDNAKNTLYLQMNSLKPEDSAMYRCA
AWTSDWSVAYWGQGTQVTVSSTSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQ
ALPPRGSGATNFSLLKQAGDVEENPGPMRISKPHLRSISIQCYLCLLLNSHFLTEAGIHVFILG
CFSAGLPKTEANWVNVISDLKKIEDLIQSMHIDATLGTESDVHPSCKVTAMKCFLLELQVIS
LESGDASIHDTVENLIILANNSLSSNGNVTESGCKECEELEEKNIKEFLQSFVHIVQMFINTS
SEQ ID NO: 34. sIL-15 m4 armored BCMA CAR
MALPVTALLLPLALLLHAARPAVQLVESGGGLVQAGDSLRLTCTASGRAFSTYFMAWFRQ
APGKEREFVAGIAWSGGSTAYADSVKGRFTISRDNAKNTVYLQMNSLKSEDTAVYYCASR
GIEVEEFGAWGQGTQVTVSSGGGGSQVQLEESGGGSVQAGGSLRLSCAYTYSTYSNYYMG
WFREAPGKARTSVAIISSDTTITYKDAVKGRFTISKDNAKNTLYLQMNSLKPEDSAMYRCA
AWTSDWSVAYWGQGTQVTVSSTSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQ
ALPPRGSGATNFSLLKQAGDVEENPGPMRISKPHLRSISIQCYLCLLLNSHFLTEAGIHVFILG
CFSAGLPKTEANWVNVISELKKIEDLIQSMHIDATLYTESDVHPSCKVTAMKCFLLELQVIS
LESGDASIHDTVENLIILANNSLSSNGNVTESGCKECEELEEKNIKEFLQSFVHIVQMFINTS
SEQ ID NO: 35. sIL-15 m5 armored BCMA CAR
MALPVTALLLPLALLLHAARPAVQLVESGGGLVQAGDSLRLTCTASGRAFSTYFMAWFRQ
APGKEREFVAGIAWSGGSTAYADSVKGRFTISRDNAKNTVYLQMNSLKSEDTAVYYCASR
GIEVEEFGAWGQGTQVTVSSGGGGSQVQLEESGGGSVQAGGSLRLSCAYTYSTYSNYYMG
WFREAPGKARTSVAIISSDTTITYKDAVKGRFTISKDNAKNTLYLQMNSLKPEDSAMYRCA
AWTSDWSVAYWGQGTQVTVSSTSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQ
ALPPRGSGATNFSLLKQAGDVEENPGPMRISKPHLRSISIQCYLCLLLNSHFLTEAGIHVFILG
CFSAGLPKTEANWVNVISDLKKIEDLIQSMHIDATLYTESDVHPSCKVTAMKCFLLELQVIS
LESGDASIHETVENLIILANNSLSSNGNVTESGCKECEELEEKNIKEFLQSFVHIVQMFINTS
SEQ ID NO: 36. sIL-15 m6 armored BCMA CAR
MALPVTALLLPLALLLHAARPAVQLVESGGGLVQAGDSLRLTCTASGRAFSTYFMAWFRQ
APGKEREFVAGIAWSGGSTAYADSVKGRFTISRDNAKNTVYLQMNSLKSEDTAVYYCASR
GIEVEEFGAWGQGTQVTVSSGGGGSQVQLEESGGGSVQAGGSLRLSCAYTYSTYSNYYMG
WFREAPGKARTSVAIISSDTTITYKDAVKGRFTISKDNAKNTLYLQMNSLKPEDSAMYRCA
AWTSDWSVAYWGQGTQVTVSSTSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQ
ALPPRGSGATNFSLLKQAGDVEENPGPMRISKPHLRSISIQCYLCLLLNSHFLTEAGIHVFILG
CFSAGLPKTEANWVNVISDLKKIEDLIQSMHIDATLYTESDVHPSCKVTAMKCFLLELQVIS
LESGDASIHDGVENLIILANNSLSSNGNVTESGCKECEELEEKNIKEFLQSFVHIVQMFINTS
SEQ ID NO: 37. sIL-15 m7 armored BCMA CAR
MALPVTALLLPLALLLHAARPAVQLVESGGGLVQAGDSLRLTCTASGRAFSTYFMAWFRQ
APGKEREFVAGIAWSGGSTAYADSVKGRFTISRDNAKNTVYLQMNSLKSEDTAVYYCASR
GIEVEEFGAWGQGTQVTVSSGGGGSQVQLEESGGGSVQAGGSLRLSCAYTYSTYSNYYMG
WFREAPGKARTSVAIISSDTTITYKDAVKGRFTISKDNAKNTLYLQMNSLKPEDSAMYRCA
AWTSDWSVAYWGQGTQVTVSSTSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQ
ALPPRGSGATNFSLLKQAGDVEENPGPMRISKPHLRSISIQCYLCLLLNSHFLTEAGIHVFILG
CFSAGLPKTEANWVNVISDLKKIEDLIQSMHIDATLYTESDVHPSCKVTAMKCFLLELQVIS
LESGDASIHDIVENLIILANNSLSSNGNVTESGCKECEELEEKNIKEFLQSFVHIVQMFINTS
SEQ ID NO: 38. sIL-15 m8 armored BCMA CAR
MALPVTALLLPLALLLHAARPAVQLVESGGGLVQAGDSLRLTCTASGRAFSTYFMAWFRQ
APGKEREFVAGIAWSGGSTAYADSVKGRFTISRDNAKNTVYLQMNSLKSEDTAVYYCASR
GIEVEEFGAWGQGTQVTVSSGGGGSQVQLEESGGGSVQAGGSLRLSCAYTYSTYSNYYMG
WFREAPGKARTSVAIISSDTTITYKDAVKGRFTISKDNAKNTLYLQMNSLKPEDSAMYRCA
AWTSDWSVAYWGQGTQVTVSSTSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQ
ALPPRGSGATNFSLLKQAGDVEENPGPMRISKPHLRSISIQCYLCLLLNSHFLTEAGIHVFILG
CFSAGLPKTEANWVNVISDLKKIEDLIQSMHIDATLYTESDVHPSCKVTAMKCFLLELQVIS
LESGDASIHDTVENLIILANNSLSSNGNVTESGCKECKELEEKNIKEFLQSFVHIVQMFINTS
SEQ ID NO: 39. mbIL-15 wt armored BCMA CAR
MALPVTALLLPLALLLHAARPAVQLVESGGGLVQAGDSLRLTCTASGRAFSTYFMAWFRQ
APGKEREFVAGIAWSGGSTAYADSVKGRFTISRDNAKNTVYLQMNSLKSEDTAVYYCASR
GIEVEEFGAWGQGTQVTVSSGGGGSQVQLEESGGGSVQAGGSLRLSCAYTYSTYSNYYMG
WFREAPGKARTSVAIISSDTTITYKDAVKGRFTISKDNAKNTLYLQMNSLKPEDSAMYRCA
AWTSDWSVAYWGQGTQVTVSSTSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQ
ALPPRGSGATNFSLLKQAGDVEENPGPMYRMQLLSCIALSLALVTNSNWVNVISDLKKIED
LIQSMHIDATLYTESDVHPSCKVTAMKCFLLELQVISLESGDASIHDTVENLIILANNSLSSN
GNVTESGCKECEELEEKNIKEFLQSFVHIVQMFINTSGGGGSGGGGSGGGGSVAISTSTVLL
CGLSAVSLLACYL
SEQ ID NO: 40. GS linker 1
GGGGSGGGGSGGGGSGGGGS
SEQ ID NO: 41. GS linker 2
GGGGS
SEQ ID NO: 42. sIL-15 m9 armored BCMA CAR
MALPVTALLLPLALLLHAARPAVQLVESGGGLVQAGDSLRLTCTASGRAFSTYFMAWFRQ
APGKEREFVAGIAWSGGSTAYADSVKGRFTISRDNAKNTVYLQMNSLKSEDTAVYYCASR
GIEVEEFGAWGQGTQVTVSSGGGGSQVQLEESGGGSVQAGGSLRLSCAYTYSTYSNYYMG
WFREAPGKARTSVAIISSDTTITYKDAVKGRFTISKDNAKNTLYLQMNSLKPEDSAMYRCA
AWTSDWSVAYWGQGTQVTVSSTSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQ
ALPPRGSGATNFSLLKQAGDVEENPGPMRISKPHLRSISIQCYLCLLLNSHFLTEAGIHVFILG
CFSAGLPKTEANWVNVISDLKKIEDLIQSMHIDATLYTESDVHPSCKVTAMKCFLLELQVIS
LESGDADIHDTVENLIILANNSLSSNGNVTESGCKECEELEEKNIKEFLQSFVHIVQMFINTS
SEQ ID NO: 43. sIL-15 m10 armored BCMA CAR
MALPVTALLLPLALLLHAARPAVQLVESGGGLVQAGDSLRLTCTASGRAFSTYFMAWFRQ
APGKEREFVAGIAWSGGSTAYADSVKGRFTISRDNAKNTVYLQMNSLKSEDTAVYYCASR
GIEVEEFGAWGQGTQVTVSSGGGGSQVQLEESGGGSVQAGGSLRLSCAYTYSTYSNYYMG
WFREAPGKARTSVAIISSDTTITYKDAVKGRFTISKDNAKNTLYLQMNSLKPEDSAMYRCA
AWTSDWSVAYWGQGTQVTVSSTSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQ
ALPPRGSGATNFSLLKQAGDVEENPGPMRISKPHLRSISIQCYLCLLLNSHFLTEAGIHVFILG
CFSAGLPKTEANWVNVISDLKKIEDLIQSMHIDATLYTESDVHPSCKVTAMKCFLLELQVIS
LESGDASIHDQVENLIILANNSLSSNGNVTESGCKECEELEEKNIKEFLQSFVHIVQMFINTS
SEQ ID NO: 44. sIL-15 m11 armored BCMA CAR
MALPVTALLLPLALLLHAARPAVQLVESGGGLVQAGDSLRLTCTASGRAFSTYFMAWFRQ
APGKEREFVAGIAWSGGSTAYADSVKGRFTISRDNAKNTVYLQMNSLKSEDTAVYYCASR
GIEVEEFGAWGQGTQVTVSSGGGGSQVQLEESGGGSVQAGGSLRLSCAYTYSTYSNYYMG
WFREAPGKARTSVAIISSDTTITYKDAVKGRFTISKDNAKNTLYLQMNSLKPEDSAMYRCA
AWTSDWSVAYWGQGTQVTVSSTSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQ
ALPPRGSGATNFSLLKQAGDVEENPGPMRISKPHLRSISIQCYLCLLLNSHFLTEAGIHVFILG
CFSAGLPKTEANWVNVISDLKKIEDLIQSMHIDATLYTESDVHPSCKVTAMKCFLLELQVIS
LESGDASIHDVVENLIILANNSLSSNGNVTESGCKECEELEEKNIKEFLQSFVHIVQMFINTS
SEQ ID NO: 45. sIL-15 m12 armored BCMA CAR
MALPVTALLLPLALLLHAARPAVQLVESGGGLVQAGDSLRLTCTASGRAFSTYFMAWFRQ
APGKEREFVAGIAWSGGSTAYADSVKGRFTISRDNAKNTVYLQMNSLKSEDTAVYYCASR
GIEVEEFGAWGQGTQVTVSSGGGGSQVQLEESGGGSVQAGGSLRLSCAYTYSTYSNYYMG
WFREAPGKARTSVAIISSDTTITYKDAVKGRFTISKDNAKNTLYLQMNSLKPEDSAMYRCA
AWTSDWSVAYWGQGTQVTVSSTSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQ
ALPPRGSGATNFSLLKQAGDVEENPGPMRISKPHLRSISIQCYLCLLLNSHFLTEAGIHVFILG
CFSAGLPKTEANWVNVISDLKKIEDLIQSMHIDATLYTESDVHPSCKVTAMKCFLLELQVIS
LESGDASIHDPVENLIILANNSLSSNGNVTESGCKECEELEEKNIKEFLQSFVHIVQMFINTS
SEQ ID NO: 46. sIL-15 m13 armored BCMA CAR
MALPVTALLLPLALLLHAARPAVQLVESGGGLVQAGDSLRLTCTASGRAFSTYFMAWFRQ
APGKEREFVAGIAWSGGSTAYADSVKGRFTISRDNAKNTVYLQMNSLKSEDTAVYYCASR
GIEVEEFGAWGQGTQVTVSSGGGGSQVQLEESGGGSVQAGGSLRLSCAYTYSTYSNYYMG
WFREAPGKARTSVAIISSDTTITYKDAVKGRFTISKDNAKNTLYLQMNSLKPEDSAMYRCA
AWTSDWSVAYWGQGTQVTVSSTSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQ
ALPPRGSGATNFSLLKQAGDVEENPGPMRISKPHLRSISIQCYLCLLLNSHFLTEAGIHVFILG
CFSAGLPKTEANWVNVISDLKKIEDLIQSMHIDATLYTESDVHPSCKVTAMKCFLLELQVIS
LESGDASIHDLVENLIILANNSLSSNGNVTESGCKECEELEEKNIKEFLQSFVHIVQMFINTS
SEQ ID NO: 47. sIL-15 m14 armored BCMA CAR
MALPVTALLLPLALLLHAARPAVQLVESGGGLVQAGDSLRLTCTASGRAFSTYFMAWFRQ
APGKEREFVAGIAWSGGSTAYADSVKGRFTISRDNAKNTVYLQMNSLKSEDTAVYYCASR
GIEVEEFGAWGQGTQVTVSSGGGGSQVQLEESGGGSVQAGGSLRLSCAYTYSTYSNYYMG
WFREAPGKARTSVAIISSDTTITYKDAVKGRFTISKDNAKNTLYLQMNSLKPEDSAMYRCA
AWTSDWSVAYWGQGTQVTVSSTSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQ
ALPPRGSGATNFSLLKQAGDVEENPGPMRISKPHLRSISIQCYLCLLLNSHFLTEAGIHVFILG
CFSAGLPKTEANWVNVISDLKKIEDLIQSMHIDATLYTESDVHPSCKVTAMKCFLLELQVIS
LESGDASIHDAVENLIILANNSLSSNGNVTESGCKECEELEEKNIKEFLQSFVHIVQMFINTS
SEQ ID NO: 48. sIL-15 m15 armored BCMA CAR
MALPVTALLLPLALLLHAARPAVQLVESGGGLVQAGDSLRLTCTASGRAFSTYFMAWFRQ
APGKEREFVAGIAWSGGSTAYADSVKGRFTISRDNAKNTVYLQMNSLKSEDTAVYYCASR
GIEVEEFGAWGQGTQVTVSSGGGGSQVQLEESGGGSVQAGGSLRLSCAYTYSTYSNYYMG
WFREAPGKARTSVAIISSDTTITYKDAVKGRFTISKDNAKNTLYLQMNSLKPEDSAMYRCA
AWTSDWSVAYWGQGTQVTVSSTSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQ
ALPPRGSGATNFSLLKQAGDVEENPGPMRISKPHLRSISIQCYLCLLLNSHFLTEAGIHVFILG
CFSAGLPKTEANWVNVISDLKKIEDLIQSMHIDATLYTESDVHPSCKVTAMKCFLLELQVIS
LESGDASIHDSVENLIILANNSLSSNGNVTESGCKECEELEEKNIKEFLQSFVHIVQMFINTS
SEQ ID NO: 49. sIL-15 m16 armored BCMA CAR
MALPVTALLLPLALLLHAARPAVQLVESGGGLVQAGDSLRLTCTASGRAFSTYFMAWFRQ
APGKEREFVAGIAWSGGSTAYADSVKGRFTISRDNAKNTVYLQMNSLKSEDTAVYYCASR
GIEVEEFGAWGQGTQVTVSSGGGGSQVQLEESGGGSVQAGGSLRLSCAYTYSTYSNYYMG
WFREAPGKARTSVAIISSDTTITYKDAVKGRFTISKDNAKNTLYLQMNSLKPEDSAMYRCA
AWTSDWSVAYWGQGTQVTVSSTSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQ
ALPPRGSGATNFSLLKQAGDVEENPGPMRISKPHLRSISIQCYLCLLLNSHFLTEAGIHVFILG
CFSAGLPKTEANWVNVISDLKKIEDLIQSMHIDATLYTESDVHPSCKVTAMKCFLLELQVIS
LESGDASIHDYVENLIILANNSLSSNGNVTESGCKECEELEEKNIKEFLQSFVHIVQMFINTS
SEQ ID NO: 50. IL15RaED
ITCPPPMSVEHADIWVKSYSLYSRERYICNSGFKRKAGTSSLTECVLNKATNVAHWTTPSL
KCIRDPALVHQRPAPPSTVTTAGVTPQPESLSPSGKEPAASSPSSNNTAATTAAIVPGSQLMP
SKSPSTGTTEISSHESSHGTPSQTTAKNWELTASASHQPPGVYPQGHSDTT
SEQ ID NO: 51. IL15RaED short(s)
ITCPPPMSVEHADIWVKSYSLYSRERYICNSGFKRKAGTSSLTECVLNKATNVAHWTTPSL
KCIR
SEQ ID NO: 52. IL15Rb
AVNGTSQFTCFYNSRANISCVWSQDGALQDTSCQVHAWPDRRRWNQTCELLPVSQASWA
CNLILGAPDSQKLTTVDIVTLRVLCREGVRWRVMAIQDFKPFENLRLMAPISLQVVHVETH
RCNISWEISQASHYFERHLEFEARTLSPGHTWEEAPLLTLKQKQEWICLETLTPDTQYEFQV
RVKPLQGEFTTWSPWSQPLAFRTKPAALGKDTIPWLGHLLVGLSGAFGFIILVYLLINCRNT
GPWLKKVLKCNTPDPSKFFSQLSSEHGGDVQKWLSSPFPSSSFSPGGLAPEISPLEVLERDK
VTQLLLQQDKVPEPASLSSNHSLTSCFTNQGYFFFHLPDALEIEACQVYFTYDPYSEEDPDE
GVAGAPTGSSPQPLQPLSGEDDAYCTFPSRDDLLLFSPSLLGGPSPPSTAPGGSGAGEERMPP
SLQERVPRDWDPQPLGPPTPGVPDLVDFQPPPELVLREAGEEVPDAGPREGVSFPWSRPPG
QGEFRALNARLPLNTDAYLSLQELQGQDPTHLV
SEQ ID NO: 53. gc, common gamma chain
MLKPSLPFTSLLFLQLPLLGVGLNTTILTPNGNEDTTADFFLTTMPTDSLSVSTLPLPEVQCF
VFNVEYMNCTWNSSSEPQPTNLTLHYWYKNSDNDKVQKCSHYLFSEEITSGCQLQKKEIH
LYQTFVVQLQDPREPRRQATQMLKLQNLVIPWAPENLTLHKLSESQLELNWNNRFLNHCL
EHLVQYRTDWDHSWTEQSVDYRHKFSLPSVDGQKRYTFRVRSRFNPLCGSAQHWSEWSH
PIHWGSNTSKENPFLFALEAVVISVGSMGLIISLLCVYFWLERTMPRIPTLKNLEDLVTEYHG
NFSAWSGVSKGLAESLQPDYSERLCLVSEIPPKGGALGEGPGASPCNQHSPYWAPPCYTLK
PET
SEQ ID NO: 54. IL15Ra
ITCPPPMSVEHADIWVKSYSLYSRERYICNSGFKRKAGTSSLTECVLNKATNVAHWTTPSL
KCIRDPALVHQRPAPPSTVTTAGVTPQPESLSPSGKEPAASSPSSNNTAATTAAIVPGSQLMP
SKSPSTGTTEISSHESSHGTPSQTTAKNWELTASASHQPPGVYPQGHSDTTVAISTSTVLLCG
LSAVSLLACYLKSRQTPPLASVEMEAMEALPVTWGTSSRDEDLENCSHHL
SEQ ID NO: 55. IL15RaTM
VAISTSTVLLCGLSAVSLLACYL
SEQ ID NO: 56. hIL-15-P2A-EGFP
MRISKPHLRSISIQCYLCLLLNSHFLTEAGIHVFILGCFSAGLPKTEANWVNVISDLKKIEDLI
QSMHIDATLYTESDVHPSCKVTAMKCFLLELQVISLESGDASIHDTVENLIILANNSLSSNGN
VTESGCKECEELEEKNIKEFLQSFVHIVQMFINTSGSGATNFSLLKQAGDVEENPGPMVSKG
EELFTGVVPILVELDGDVNGHKFSVSGEGEGDATYGKLTLKFICTTGKLPVPWPTLVTTLTY
GVQCFSRYPDHMKQHDFFKSAMPEGYVQERTIFFKDDGNYKTRAEVKFEGDTLVNRIELK
GIDFKEDGNILGHKLEYNYNSHNVYIMADKQKNGIKVNFKIRHNIEDGSVQLADHYQQNTP
IGDGPVLLPDNHYLSTQSALSKDPNEKRDHMVLLEFVTAAGITLGMDELYK
SEQ ID NO: 57. sIL-15 m6-Ra
MRISKPHLRSISIQCYLCLLLNSHFLTEAGIHVFILGCFSAGLPKTEANWVNVISDLKKIEDLI
QSMHIDATLYTESDVHPSCKVTAMKCFLLELQVISLESGDASIHDGVENLIILANNSLSSNG
NVTESGCKECEELEEKNIKEFLQSFVHIVQMFINTSGGGGSGGGGSGGGGSITCPPPMSVEH
ADIWVKSYSLYSRERYICNSGFKRKAGTSSLTECVLNKATNVAHWTTPSLKCIRDPALVHQ
RPAPPSTVTTAGVTPQPESLSPSGKEPAASSPSSNNTAATTAAIVPGSQLMPSKSPSTGTTEIS
SHESSHGTPSQTTAKNWELTASASHQPPGVYPQGHSDTT
SEQ ID NO: 58. sIL-15 m6-sRa
MRISKPHLRSISIQCYLCLLLNSHFLTEAGIHVFILGCFSAGLPKTEANWVNVISDLKKIEDLI
QSMHIDATLYTESDVHPSCKVTAMKCFLLELQVISLESGDASIHDGVENLIILANNSLSSNG
NVTESGCKECEELEEKNIKEFLQSFVHIVQMFINTSGGGGSGGGGGGGGSITCPPPMSVEH
ADIWVKSYSLYSRERYICNSGFKRKAGTSSLTECVLNKATNVAHWTTPSLKCIR
SEQ ID NO: 59. mb-1 IL-15 m6
MRISKPHLRSISIQCYLCLLLNSHFLTEAGIHVFILGCFSAGLPKTEANWVNVISDLKKIEDLI
QSMHIDATLYTESDVHPSCKVTAMKCFLLELQVISLESGDASIHDGVENLIILANNSLSSNG
NVTESGCKECEELEEKNIKEFLQSFVHIVQMFINTSGGGGSGGGGSGGGGSITCPPPMSVEH
ADIWVKSYSLYSRERYICNSGFKRKAGTSSLTECVLNKATNVAHWTTPSLKCIRGGGGSGG
GGSGGGGSVAISTSTVLLCGLSAVSLLACYL
SEQ ID NO: 60. mb-4 IL-15 m6
MAAPALSWRLPLLILLLPLATSWASANWVNVISDLKKIEDLIQSMHIDATLYTESDVHPSCK
VTAMKCFLLELQVISLESGDASIHDGVENLIILANNSLSSNGNVTESGCKECEELEEKNIKEF
LQSFVHIVQMFINTSGGGGSGGGGSGGGGSGGGGSAVNGTSQFTCFYNSRANISCVWSQD
GALQDTSCQVHAWPDRRRWNQTCELLPVSQASWACNLILGAPDSQKLTTVDIVTLRVLCR
EGVRWRVMAIQDFKPFENLRLMAPISLQVVHVETHRCNISWEISQASHYFERHLEFEARTLS
PGHTWEEAPLLTLKQKQEWICLETLTPDTQYEFQVRVKPLQGEFTTWSPWSQPLAFRTKPA
ALGKDTIPWLGHLLVGLSGAFGFIILVYLLINCRNTGPWLKKVLKCNTPDPSKFFSQLSSEH
GGDVQKWLSSPFPSSSFSPGGLAPEISPLEVLERDKVTQLLLQQDKVPEPASLSSNHSLTSCF
TNQGYFFFHLPDALEIEACQVYFTYDPYSEEDPDEGVAGAPTGSSPQPLQPLSGEDDAYCTF
PSRDDLLLFSPSLLGGPSPPSTAPGGSGAGEERMPPSLQERVPRDWDPQPLGPPTPGVPDLV
DFQPPPELVLREAGEEVPDAGPREGVSFPWSRPPGQGEFRALNARLPLNTDAYLSLQELQG
QDPTHLVGSGATNFSLLKQAGDVEENPGPMLKPSLPFTSLLFLQLPLLGVGLNTTILTPNGN
EDTTADFFLTTMPTDSLSVSTLPLPEVQCFVFNVEYMNCTWNSSSEPQPTNLTLHYWYKNS
DNDKVQKCSHYLFSEEITSGCQLQKKEIHLYQTFVVQLQDPREPRRQATQMLKLQNLVIPW
APENLTLHKLSESQLELNWNNRFLNHCLEHLVQYRTDWDHSWTEQSVDYRHKFSLPSVDG
QKRYTFRVRSRFNPLCGSAQHWSEWSHPIHWGSNTSKENPFLFALEAVVISVGSMGLIISLL
CVYFWLERTMPRIPTLKNLEDLVTEYHGNFSAWSGVSKGLAESLQPDYSERLCLVSEIPPK
GGALGEGPGASPCNQHSPYWAPPCYTLKPET
SEQ ID NO: 61. mb-5 IL-15 m6
MAPRRARGCRTLGLPALLLLLLLRPPATRGNWVNVISDLKKIEDLIQSMHIDATLYTESDVH
PSCKVTAMKCFLLELQVISLESGDASIHDGVENLIILANNSLSSNGNVTESGCKECEELEEKN
IKEFLQSFVHIVQMFINTSGGGGSGGGGSGGGGSITCPPPMSVEHADIWVKSYSLYSRERYIC
NSGFKRKAGTSSLTECVLNKATNVAHWTTPSLKCIRDPALVHQRPAPPSTVTTAGVTPQPE
SLSPSGKEPAASSPSSNNTAATTAAIVPGSQLMPSKSPSTGTTEISSHESSHGTPSQTTAKNW
ELTASASHQPPGVYPQGHSDTTVAISTSTVLLCGLSAVSLLACYLKSRQTPPLASVEMEAME
ALPVTWGTSSRDEDLENCSHHL
SEQ ID NO: 62. mb-6 IL-15 m6
MAAPALSWRLPLLILLLPLATSWASANWVNVISDLKKIEDLIQSMHIDATLYTESDVHPSCK
VTAMKCFLLELQVISLESGDASIHDGVENLIILANNSLSSNGNVTESGCKECEELEEKNIKEF
LQSFVHIVQMFINTSGGGGSGGGGSGGGGSAVNGTSQFTCFYNSRANISCVWSQDGALQD
TSCQVHAWPDRRRWNQTCELLPVSQASWACNLILGAPDSQKLTTVDIVTLRVLCREGVRW
RVMAIQDFKPFENLRLMAPISLQVVHVETHRCNISWEISQASHYFERHLEFEARTLSPGHTW
EEAPLLTLKQKQEWICLETLTPDTQYEFQVRVKPLQGEFTTWSPWSQPLAFRTKPAALGKD
TIPWLGHLLVGLSGAFGFIILVYLLINCRNTGPWLKKVLKCNTPDPSKFFSQLSSEHGGDVQ
KWLSSPFPSSSFSPGGLAPEISPLEVLERDKVTQLLLQQDKVPEPASLSSNHSLTSCFTNQGY
FFFHLPDALEIEACQVYFTYDPYSEEDPDEGVAGAPTGSSPQPLQPLSGEDDAYCTFPSRDD
LLLFSPSLLGGPSPPSTAPGGSGAGEERMPPSLQERVPRDWDPQPLGPPTPGVPDLVDFQPPP
ELVLREAGEEVPDAGPREGVSFPWSRPPGQGEFRALNARLPLNTDAYLSLQELQGQDPTHL
V
SEQ ID NO: 63. mb-7 IL-15 m6
MLKPSLPFTSLLFLQLPLLGVGNWVNVISDLKKIEDLIQSMHIDATLYTESDVHPSCKVTAM
KCFLLELQVISLESGDASIHDGVENLIILANNSLSSNGNVTESGCKECEELEEKNIKEFLQSFV
HIVQMFINTSGGGGSGGGGSGGGGSLNTTILTPNGNEDTTADFFLTTMPTDSLSVSTLPLPE
VQCFVFNVEYMNCTWNSSSEPQPTNLTLHYWYKNSDNDKVQKCSHYLFSEEITSGCQLQK
KEIHLYQTFVVQLQDPREPRRQATQMLKLQNLVIPWAPENLTLHKLSESQLELNWNNRFLN
HCLEHLVQYRTDWDHSWTEQSVDYRHKFSLPSVDGQKRYTFRVRSRFNPLCGSAQHWSE
WSHPIHWGSNTSKENPFLFALEAVVISVGSMGLIISLLCVYFWLERTMPRIPTLKNLEDLVTE
YHGNFSAWSGVSKGLAESLQPDYSERLCLVSEIPPKGGALGEGPGASPCNQHSPYWAPPCY
TLKPET
SEQ ID NO: 64. mb-8 IL-15 m6
MYRMQLLSCIALSLALVTNSNWVNVISDLKKIEDLIQSMHIDATLYTESDVHPSCKVTAMK
CFLLELQVISLESGDASIHDGVENLIILANNSLSSNGNVTESGCKECEELEEKNIKEFLQSFVH
IVQMFINTSGGGGSGGGGSGGGGSVAISTSTVLLCGLSAVSLLACYL
SEQ ID NO: 65. mb-2 IL-15 m6 armored BCMA CAR
MRISKPHLRSISIQCYLCLLLNSHFLTEAGIHVFILGCFSAGLPKTEANWVNVISDLKKIEDLI
QSMHIDATLYTESDVHPSCKVTAMKCFLLELQVISLESGDASIHDGVENLIILANNSLSSNG
NVTESGCKECEELEEKNIKEFLQSFVHIVQMFINTSGGGGSGGGGSGGGGSGGGGSAVQLV
ESGGGLVQAGDSLRLTCTASGRAFSTYFMAWFRQAPGKEREFVAGIAWSGGSTAYADSVK
GRFTISRDNAKNTVYLQMNSLKSEDTAVYYCASRGIEVEEFGAWGQGTQVTVSSGGGGSQ
VQLEESGGGSVQAGGSLRLSCAYTYSTYSNYYMGWFREAPGKARTSVAIISSDTTITYKDA
VKGRFTISKDNAKNTLYLQMNSLKPEDSAMYRCAAWTSDWSVAYWGQGTQVTVSSTSTT
TPAPRPPTPAPTIASQPLSLRPEACRPAAGGAVHTRGLDFACDIYIWAPLAGTCGVLLLSLVI
TLYCKRGRKKLLYIFKQPFMRPVQTTQEEDGCSCRFPEEEEGGCELRVKFSRSADAPAYQQ
GQNQLYNELNLGRREEYDVLDKRRGRDPEMGGKPRRKNPQEGLYNELQKDKMAEAYSEI
GMKGERRRGKGHDGLYQGLSTATKDTYDALHMQALPPR
SEQ ID NO: 66. mb-3 IL-15 m6 armored BCMA CAR
MALPVTALLLPLALLLHAARPAVQLVESGGGLVQAGDSLRLTCTASGRAFSTYFMAWFRQ
APGKEREFVAGIAWSGGSTAYADSVKGRFTISRDNAKNTVYLQMNSLKSEDTAVYYCASR
GIEVEEFGAWGQGTQVTVSSGGGGSQVQLEESGGGSVQAGGSLRLSCAYTYSTYSNYYMG
WFREAPGKARTSVAIISSDTTITYKDAVKGRFTISKDNAKNTLYLQMNSLKPEDSAMYRCA
AWTSDWSVAYWGQGTQVTVSSGGGGSGGGGSGGGGSNWVNVISDLKKIEDLIQSMHIDA
TLYTESDVHPSCKVTAMKCFLLELQVISLESGDASIHDGVENLIILANNSLSSNGNVTESGCK
ECEELEEKNIKEFLQSFVHIVQMFINTSGGGGSGGGGSGGGGSTSTTTPAPRPPTPAPTIASQP
LSLRPEACRPAAGGAVHTRGLDFACDIYIWAPLAGTCGVLLLSLVITLYCKRGRKKLLYIFK
QPFMRPVQTTQEEDGCSCRFPEEEEGGCELRVKFSRSADAPAYQQGQNQLYNELNLGRRE
EYDVLDKRRGRDPEMGGKPRRKNPQEGLYNELQKDKMAEAYSEIGMKGERRRGKGHDG
LYQGLSTATKDTYDALHMQALPPR
SEQ ID NO: 67 GS linker 3
(GS)ₙ
SEQ ID NO: 68 GS linker 4
(GSGGS)ₙ
SEQ ID NO: 69 GS linker 5
(GGGS)ₙ
SEQ ID NO: 70 IL15 Signal Peptide (SP) and pro-peptide
MRISKPHLRSISIQCYLCLLLNSHFLTEAGIHVFILGCFSAGLPKTEA
SEQ ID NO: 71 IL-15Rβ SP
MAAPALSWRLPLLILLLPLATSWASA
SEQ ID NO: 72 IL-15Rα SP
MAPRRARGCRTLGLPALLLLLLLRPPATRG
SEQ ID NO: 73 SP of common gamma chain
MLKPSLPFTSLLFLQLPLLGVG
SEQ ID NO: 74 SP of IL-2
MYRMQLLSCIALSLALVINS
SEQ ID NO: 75 mb-9 IL-15 m4 armored BCMA CAR
MALPVTALLLPLALLLHAARPAVQLVESGGGLVQAGDSLRLTCTASGRAFSTYFMAWFRQ
APGKEREFVAGIAWSGGSTAYADSVKGRFTISRDNAKNTVYLQMNSLKSEDTAVYYCASR
GIEVEEFGAWGQGTQVTVSSGGGGSQVQLEESGGGSVQAGGSLRLSCAYTYSTYSNYYMG
WFREAPGKARTSVAIISSDTTITYKDAVKGRFTISKDNAKNTLYLQMNSLKPEDSAMYRCA
AWTSDWSVAYWGQGTQVTVSSGGGGSGGGGSGGGGSNWVNVISELKKIEDLIQSMHIDA
TLYTESDVHPSCKVTAMKCFLLELQVISLESGDASIHDTVENLIILANNSLSSNGNVTESGCK
ECEELEEKNIKEFLQSFVHIVQMFINTSGGGGSGGGGSGGGGSTSTTTPAPRPPTPAPTIASQP
LSLRPEACRPAAGGAVHTRGLDFACDIYIWAPLAGTCGVLLLSLVITLYCKRGRKKLLYIFK
QPFMRPVQTTQEEDGCSCRFPEEEEGGCELRVKFSRSADAPAYQQGQNQLYNELNLGRRE
EYDVLDKRRGRDPEMGGKPRRKNPQEGLYNELQKDKMAEAYSEIGMKGERRRGKGHDG
LYQGLSTATKDTYDALHMQALPPR
SEQ ID NO: 76 mb-10 IL-15 m4
MAAPALSWRLPLLILLLPLATSWASANWVNVISELKKIEDLIQSMHIDATLYTESDVHPSCK
VTAMKCFLLELQVISLESGDASIHDTVENLIILANNSLSSNGNVTESGCKECEELEEKNIKEF
LQSFVHIVQMFINTSGGGGSGGGGSGGGGSGGGGSAVNGTSQFTCFYNSRANISCVWSQD
GALQDTSCQVHAWPDRRRWNQTCELLPVSQASWACNLILGAPDSQKLTTVDIVTLRVLCR
EGVRWRVMAIQDFKPFENLRLMAPISLQVVHVETHRCNISWEISQASHYFERHLEFEARTLS
PGHTWEEAPLLTLKQKQEWICLETLTPDTQYEFQVRVKPLQGEFTTWSPWSQPLAFRTKPA
ALGKDTIPWLGHLLVGLSGAFGFIILVYLLINCRNTGPWLKKVLKCNTPDPSKFFSQLSSEH
GGDVQKWLSSPFPSSSFSPGGLAPEISPLEVLERDKVTQLLLQQDKVPEPASLSSNHSLTSCF
TNQGYFFFHLPDALEIEACQVYFTYDPYSEEDPDEGVAGAPTGSSPQPLQPLSGEDDAYCTF
PSRDDLLLFSPSLLGGPSPPSTAPGGSGAGEERMPPSLQERVPRDWDPQPLGPPTPGVPDLV
DFQPPPELVLREAGEEVPDAGPREGVSFPWSRPPGQGEFRALNARLPLNTDAYLSLQELQG
QDPTHLVGSGATNFSLLKQAGDVEENPGPMLKPSLPFTSLLFLQLPLLGVGLNTTILTPNGN
EDTTADFFLTTMPTDSLSVSTLPLPEVQCFVFNVEYMNCTWNSSSEPQPTNLTLHYWYKNS
DNDKVQKCSHYLFSEEITSGCQLQKKEIHLYQTFVVQLQDPREPRRQATQMLKLQNLVIPW
APENLTLHKLSESQLELNWNNRFLNHCLEHLVQYRTDWDHSWTEQSVDYRHKFSLPSVDG
QKRYTFRVRSRFNPLCGSAQHWSEWSHPIHWGSNTSKENPFLFALEAVVISVGSMGLIISLL
CVYFWLERTMPRIPTLKNLEDLVTEYHGNFSAWSGVSKGLAESLQPDYSERLCLVSEIPPK
GGALGEGPGASPCNQHSPYWAPPCYTLKPET
SEQ ID NO: 77 mb-11 IL-15 m4
MRISKPHLRSISIQCYLCLLLNSHFLTEAGIHVFILGCFSAGLPKTEANWVNVISELKKIEDLI
QSMHIDATLYTESDVHPSCKVTAMKCFLLELQVISLESGDASIHDTVENLIILANNSLSSNGN
VTESGCKECEELEEKNIKEFLQSFVHIVQMFINTSGGGGSGGGGSGGGGSITCPPPMSVEHA
DIWVKSYSLYSRERYICNSGFKRKAGTSSLTECVLNKATNVAHWTTPSLKCIRDPALVHQR
PAPPSTVTTAGVTPQPESLSPSGKEPAASSPSSNNTAATTAAIVPGSQLMPSKSPSTGTTEISS
HESSHGTPSQTTAKNWELTASASHQPPGVYPQGHSDTTVAISTSTVLLCGLSAVSLLACYL
KSRQTPPLASVEMEAMEALPVTWGTSSRDEDLENCSHHL
SEQ ID NO: 78 IL15 mutein 17
NWYNVISDLKKIEDLIQSMHIDATLYTESDVHPSCKVTAMKCFLLELQVISLESGDASIHDT
VENLIILANNSLSSNGNVTESGCKECEELEEKNIKEFLQSFVHIVQMFINTS
SEQ ID NO: 79 IL15 mutein 18
NWVNVISDLKKIEDLIQSMHIDATFYTESDVHPSCKVTAMKCFLLELQVISLESGDASIHDT
VENLIILANNSLSSNGNVTESGCKECEELEEKNIKEFLQSFVHIVQMFINTS
SEQ ID NO: 80 GPC3 scFv
DIVMTQSPLSLPVTPGEPASISCRSSQTIVHSNGNTYLEWYLQKPGQSPQLLIYKVSNRFSGV
PDRFSGSGSGTDFTLKISRVEAEDVGVYYCFQVSHVPYTFGGGTKLEIKGSTSGSGKPGSGE
GSTKGQVQLVQSGAEVKKPGASVKVSCKASGYTFTDYEMHWVRQATGQGLEWMGGIDP
ETGNTAYSQKFKGRVTMTRNTSISTAYMELSSLRSEDTAVYYCARYYSFAYWGQGTLVTV
SS
SEQ ID NO: 81 GPC3 CAR
MALPVTALLLPLALLLHAARPDIVMTQSPLSLPVTPGEPASISCRSSQTIVHSNGNTYLEWY
LQKPGQSPQLLIYKVSNRFSGVPDRFSGSGSGTDFTLKISRVEAEDVGVYYCFQVSHVPYTF
GGGTKLEIKGSTSGSGKPGSGEGSTKGQVQLVQSGAEVKKPGASVKVSCKASGYTFTDYE
MHWVRQATGQGLEWMGGIDPETGNTAYSQKFKGRVTMTRNTSISTAYMELSSLRSEDTA
VYYCARYYSFAYWGQGTLVTVSSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATKDTYDALHM
QALPPR
SEQ ID NO: 82 sIL-15 wt armored GPC3 CAR
MALPVTALLLPLALLLHAARPDIVMTQSPLSLPVTPGEPASISCRSSQTIVHSNGNTYLEWY
LQKPGQSPQLLIYKVSNRFSGVPDRFSGSGSGTDFTLKISRVEAEDVGVYYCFQVSHVPYTF
GGGTKLEIKGSTSGSGKPGSGEGSTKGQVQLVQSGAEVKKPGASVKVSCKASGYTFTDYE
MHWVRQATGQGLEWMGGIDPETGNTAYSQKFKGRVTMTRNTSISTAYMELSSLRSEDTA
VYYCARYYSFAYWGQGTLVTVSSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATKDTYDALHM
QALPPRGSGATNFSLLKQAGDVEENPGPMRISKPHLRSISIQCYLCLLLNSHFLTEAGIHVFIL
GCFSAGLPKTEANWVNVISDLKKIEDLIQSMHIDATLYTESDVHPSCKVTAMKCFLLELQVI
SLESGDASIHDTVENLIILANNSLSSNGNVTESGCKECEELEEKNIKEFLQSFVHIVQMFINTS
SEQ ID NO: 83 sIL-15 m17 armored GPC3 CAR
MALPVTALLLPLALLLHAARPDIVMTQSPLSLPVTPGEPASISCRSSQTIVHSNGNTYLEWY
LQKPGQSPQLLIYKVSNRFSGVPDRFSGSGSGTDFTLKISRVEAEDVGVYYCFQVSHVPYTF
GGGTKLEIKGSTSGSGKPGSGEGSTKGQVQLVQSGAEVKKPGASVKVSCKASGYTFTDYE
MHWVRQATGQGLEWMGGIDPETGNTAYSQKFKGRVTMTRNTSISTAYMELSSLRSEDTA
VYYCARYYSFAYWGQGTLVTVSSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATKDTYDALHM
QALPPRGSGATNFSLLKQAGDVEENPGPMRISKPHLRSISIQCYLCLLLNSHFLTEAGIHVFIL
GCFSAGLPKTEANWYNVISDLKKIEDLIQSMHIDATLYTESDVHPSCKVTAMKCFLLELQVI
SLESGDASIHDTVENLIILANNSLSSNGNVTESGCKECEELEEKNIKEFLQSFVHIVQMFINTS
SEQ ID NO: 84 sIL-15 m18 armored GPC3 CAR
MALPVTALLLPLALLLHAARPDIVMTQSPLSLPVTPGEPASISCRSSQTIVHSNGNTYLEWY
LQKPGQSPQLLIYKVSNRFSGVPDRFSGSGSGTDFTLKISRVEAEDVGVYYCFQVSHVPYTF
GGGTKLEIKGSTSGSGKPGSGEGSTKGQVQLVQSGAEVKKPGASVKVSCKASGYTFTDYE
MHWVRQATGQGLEWMGGIDPETGNTAYSQKFKGRVTMTRNTSISTAYMELSSLRSEDTA
VYYCARYYSFAYWGQGTLVTVSSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATKDTYDALHM
QALPPRGSGATNFSLLKQAGDVEENPGPMRISKPHLRSISIQCYLCLLLNSHFLTEAGIHVFIL
GCFSAGLPKTEANWVNVISDLKKIEDLIQSMHIDATFYTESDVHPSCKVTAMKCFLLELQVI
SLESGDASIHDTVENLIILANNSLSSNGNVTESGCKECEELEEKNIKEFLQSFVHIVQMFINTS
SEQ ID NO: 85 CD32 intracellular domain
RVKFSRSADAPAYQQGQNQLYNELNLGRREEYDVLDKRRGRDPEMGGKPQRRKNPQEGL
YNELQKDKMAEAYSEIGMKGERRRGKGHDGLYQGLSTATKDTYDALHMQALPPR

What is claimed is:

1. A modified immune cell comprising a first heterologous nucleic acid sequence encoding an IL-15 polypeptide comprising one or more amino acid substitutions at positions 8 and/or 62, wherein numbering of the amino acid residue positions is according to SEQ ID NO: 1.

2. The modified immune cell of claim 1, wherein the IL-15 polypeptide comprises an amino acid substitution at position 62.

3. The modified immune cell of claim 2, wherein the IL-15 polypeptide comprises an amino acid residue selected from the group consisting of Glycine (G), Isoleucine (I), Glutamine (Q), Valine (V), Proline (P), Leucine (L), Alanine (A), Serine (S) and Tyrosine (Y) at position 62.

4. The modified immune cell of claim 2 or 3, wherein the amino acid substitution at position 62 is selected from the group consisting of T62G, T62I, T62Q, T62V, T62P, T62L, T62A, T62S and T62Y.

5. The modified immune cell of claim 4, wherein the amino acid substitution at position 62 is T62G.

6. The modified immune cell of any one of claims 2-5, wherein the IL-15 polypeptide comprises an amino acid sequence having at least 90% sequence identity to the amino acid sequence of SEQ ID NO: 7.

7. The modified immune cell of any one of the preceding claims, wherein the IL-15 polypeptide comprises an amino acid substitution at position 8.

8. The modified immune cell of claim 7, wherein the IL-15 polypeptide comprises an amino acid residue Glutamic acid (E) at position 8.

9. The modified immune cell of claim 8, wherein the amino acid substitution at position 8 is D8E.

10. The modified immune cell of claim 8 or 9, wherein the IL-15 polypeptide comprises the amino acid sequence having at least 90% sequence identity to the amino acid sequence of SEQ ID NO: 5.

11. A modified immune cell comprising a first heterologous nucleic acid sequence encoding an IL-15 polypeptide comprising one or more amino acid substitutions at positions 3 and/or 25, wherein numbering of the amino acid residue positions is according to SEQ ID NO: 1.

12. The modified immune cell of claim 11, wherein the amino acid substitution at position 3 is V3Y and/or the amino acid substitution at position 25 is L25F.

13. The modified immune cell of claim 12, wherein the IL-15 polypeptide comprises the amino acid sequence having at least 90% sequence identity to the amino acid sequence of SEQ ID NO: 78 or 79.

14. The modified immune cell of any one of the preceding claims, wherein the one or more amino acid substitutions reduce affinity of the IL-15 polypeptide to IL-15Rβ compared to an IL-15 polypeptide that does not comprise the one or more amino acid substitutions.

15. A modified immune cell comprising a first heterologous nucleic acid sequence encoding an IL-15 polypeptide that induces secretion of an inflammatory cytokine by the modified immune cell at a level that is least 50% lower than that by a modified immune cell comprising a heterologous nucleic acid sequence encoding a wildtype TL-15 polypeptide.

16. The modified immune cell of any one of the preceding claims, wherein the IL-15 polypeptide is secreted.

17. The modified immune cell of any one of claims 1-16, wherein the IL-15 polypeptide is membrane bound.

18. The modified immune cell of claim 17, wherein the IL-15 polypeptide comprises a glycosylphosphatidylinositol (GPI)—anchoring peptide sequence.

19. The modified immune cell of claim 17, wherein the IL-15 polypeptide comprises a transmembrane domain.

20. The modified immune cell of claim 17, wherein the IL-15 polypeptide comprises a membrane anchoring domain.

21. The modified immune cell of any one of the preceding claims, wherein the IL-15 polypeptide is a fusion protein comprising an IL-15 fragment fused to a second polypeptide fragment.

22. The modified immune cell of claim 21, wherein the second polypeptide fragment is selected from the group consisting of IL-15Rα, an extracellular domain of IL-15Rα, a Sushi domain of IL-15Rα, a transmembrane domain of IL-15Rα, IL-15Rβ, common gamma chain (γc) and combinations thereof.

23. The modified immune cell of claim 22, wherein the second polypeptide fragment comprises an amino acid sequence selected from the group consisting of SEQ ID NOs: 50-55.

24. The modified immune cell of claim 17, wherein the IL-15 polypeptide comprises: (a) an antigen-binding domain; (b) an IL-15 fragment; (c) a transmembrane domain; and (d) an intracellular domain.

25. The modified immune cell of any one of claims 1-23, wherein the modified immune cell comprises a second heterologous nucleic acid sequence encoding an engineered receptor.

26. The modified immune cell of claim 25, wherein the engineered receptor is a chimeric antigen receptor (CAR).

27. The modified immune cell of claim 26, wherein the CAR is a BCMA CAR, a CD19 CAR, or a GPC3 CAR.

28. The modified immune cell of claim 25, wherein the engineered receptor is a modified T-cell receptor (TCR).

29. The modified immune cell of claim 25, wherein the engineered receptor is a T-cell antigen coupler (TAC) receptor.

30. The modified immune cell of any one of claims 25-29, wherein the first nucleic acid sequence and the second nucleic acid sequence are operably linked to the same promoter.

31. The modified immune cell of any one of claims 25-29, wherein the first nucleic acid and the second nucleic acid are operably linked to separate promoters.

32. The modified immune cell of any one of the preceding claims, wherein the modified immune cell is selected from the group consisting of a cytotoxic T cell, a helper T cell, a natural killer (NK) cell, an NK-cell, an iNK-T cell, an NK-T like cell, an αβT cell and a γδT cell.

33. The modified immune cell of claim 32, wherein the modified immune cell is an NK cell.

34. The modified immune cell of claim 32, wherein the modified immune cell is a cytotoxic T cell.

35. The modified immune cell of any one of the preceding claims, wherein the modified immune cell has reduced toxicity in vivo when administered to an individual compared to a modified immune cell that does not comprise the first heterologous nucleic acid encoding the IL-15 polypeptide.

36. A method of producing a modified immune cell, comprising: introducing into a precursor immune cell a first nucleic acid sequence encoding an IL-15 polypeptide comprising one or more amino acid substitutions at positions 8 and/or 62, wherein numbering of the amino acid residue positions is according to SEQ ID NO: 1.

37. The method of claim 36, wherein the precursor immune cell is selected from the group consisting of a cytotoxic T cell, a helper T cell, an NK cell, an NK-T cell, an iNK-T cell, an NK-T like cell, an αβT cell and a γδT cell.

38. The method of claim 36 or 37, wherein the precursor immune cell comprises an engineered receptor.

39. The method of claim 36 or 37, further comprising introducing into the precursor immune cell a second nucleic acid encoding an engineered receptor.

40. The method of claim 38 or 39, wherein the engineered receptor is a chimeric antigen receptor (CAR), a modified T-cell receptor (TCR), or a T-cell antigen coupler (TAC) receptor.

41. The method of claim 39 or 40, wherein the first nucleic acid sequence and the second nucleic acid sequence are on the same vector.

42. The method of claim 41, wherein the vector is a viral vector.

43. The method of claim 42, wherein the viral vector is selected from the group consisting of an adenoviral vector, an adeno-associated virus vector, a retroviral vector, a lentiviral vector, a herpes simplex viral vector, and derivatives thereof.

44. The method of any one of claims 36-43, further comprising isolating or enriching immune cells comprising the first and/or the second nucleic acid sequence.

45. A modified immune cell produced by the method of any one of claims 36-44.

46. A pharmaceutical composition comprising the modified immune cell of claims 1-35 and 45, and a pharmaceutically acceptable carrier.

47. A method of treating a disease in an individual, comprising administering to the individual an effective amount of the pharmaceutical composition of claim 46.

48. The method of claim 47, wherein the disease is cancer.

49. The method of claim 48, wherein the individual has a low tumor burden.

50. The method of any one of claims 47-49, wherein the method does not result in cytokine storm in the individual.

51. The method of any one of claims 47-50, wherein the individual is human.

52. A method of reducing cytokine storm in an individual receiving treatment with an immune cell comprising an engineered receptor, comprising: (a) introducing to the immune cell a heterologous nucleic acid sequence encoding an IL-15 polypeptide comprising one or more amino acid substitutions at positions 8 and/or 62, wherein numbering of the amino acid residue positions is according to SEQ ID NO: 1, thereby providing a modified immune cell; and (b) administering to the individual an effective amount of the modified immune cell.

53. An engineered IL-15 polypeptide comprising amino acid substitution D8E, T62G, V3Y and/or L25F, wherein numbering of the amino acid residue positions is according to SEQ ID NO: 1.

54. The engineered IL-15 polypeptide of claim 53, comprising an amino acid sequence having at least about 90% sequence identity to an amino acid sequence selected from the group consisting of SEQ ID NOs: 5, 7, 78 and 79.


SEQUENCE LISTING (85 sequences)

SEQ ID NO: 1 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - protein, 114 aa, synthetic construct
NWVNVISDLKKIEDLIQSMHIDLTLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - protein, 114 aa, synthetic construct
NWVNVISDLKKIEDLIQSMHIDATEYTESDVHPSCKVTAMKCFLLELQVISLESGDASIHDTVENLIILANNSLSSNGNVTESGCKECEELEEKNIKEFLQSFVHIVQMFINTS

SEQ ID NO: 4 - protein, 114 aa, synthetic construct
NWVNVISDLKKIEDLIQSMHIDATLGTESDVHPSCKVTAMKCFLLELQVISLESGDASIHDTVENLIILANNSLSSNGNVTESGCKECEELEEKNIKEFLQSFVHIVQMFINTS

SEQ ID NO: 5 - protein, 114 aa, synthetic construct
NWVNVISELKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 6 - protein, 114 aa, synthetic construct
NWVNVISDLKKIEDLIQSMHIDATLYTESDVHPSCKVTAMKCFLLELQVISLESGDASIHETVENLIILANNSLSSNGNVTESGCKECEELEEKNIKEFLQSFVHIVQMFINTS

SEQ ID NO: 7 - protein, 114 aa, synthetic construct
NWVNVISDLKKIEDLIQSMHIDATLYTESDVHPSCKVTAMKCFLLELQVISLESGDASIHDGVENLIILANNSLSSNGNVTESGCKECEELEEKNIKEFLQSFVHIVQMFINTS

SEQ ID NO: 8 - protein, 114 aa, synthetic construct
NWVNVISDLKKIEDLIQSMHIDATLYTESDVHPSCKVTAMKCFLLELQVISLESGDASIHDIVENLIILANNSLSSNGNVTESGCKECEELEEKNIKEFLQSFVHIVQMFINTS

SEQ ID NO: 9 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECKELEEKNIKEFLQSFVHIVQMFINTS

SEQ ID NO: 10 - protein, 114 aa, synthetic construct
NWVNVISDLKKIEDLIQSMHIDATLYTESDVHPSCKVTAMKCFLLELQVISLESGDADIHDTVENLIILANNSLSSNGNVTESGCKECEELEEKNIKEFLQSFVHIVQMFINTS

SEQ ID NO: 11 - protein, 114 aa, synthetic construct
NWVNVISDLKKIEDLIQSMHIDATLYTESDVHPSCKVTAMKCFLLELQVISLESGDASIHDQVENLIILANNSLSSNGNVTESGCKECEELEEKNIKEFLQSFVHIVQMFINTS

SEQ ID NO: 12 - protein, 114 aa, synthetic construct
NWVNVISDLKKIEDLIQSMHIDATLYTESDVHPSCKVTAMKCFLLELQVISLESGDASIHDVVENLIILANNSLSSNGNVTESGCKECEELEEKNIKEFLQSFVHIVQMFINTS

SEQ ID NO: 13 - protein, 114 aa, synthetic construct
NWVNVISDLKKIEDLIQSMHIDATLYTESDVHPSCKVTAMKCFLLELQVISLESGDASIHDPVENLIILANNSLSSNGNVTESGCKECEELEEKNIKEFLQSFVHIVQMFINTS

SEQ ID NO: 14 - protein, 114 aa, synthetic construct
NWVNVISDLKKIEDLIQSMHIDATLYTESDVHPSCKVTAMKCFLLELQVISLESGDASIHDLVENLIILANNSLSSNGNVTESGCKECEELEEKNIKEFLQSFVHIVQMFINTS

SEQ ID NO: 15 - protein, 114 aa, synthetic construct
NWVNVISDLKKIEDLIQSMHIDATLYTESDVHPSCKVTAMKCFLLELQVISLESGDASIHDAVENLIILANNSLSSNGNVTESGCKECEELEEKNIKEFLQSFVHIVQMFINTS

SEQ ID NO: 16 - protein, 114 aa, synthetic construct
NWVNVISDLKKIEDLIQSMHIDATLYTESDVHPSCKVTAMKCFLLELQVISLESGDASIHDSVENLIILANNSLSSNGNVTESGCKECEELEEKNIKEFLQSFVHIVQMFINTS

SEQ ID NO: 17 - protein, 114 aa, synthetic construct
NWVNVISDLKKIEDLIQSMHIDATLYTESDVHPSCKVTAMKCFLLELQVISLESGDASIHDYVENLIILANNSLSSNGNVTESGCKECEELEEKNIKEFLQSFVHIVQMFINTS

SEQ ID NO: 18 - protein, 45 aa, synthetic construct
TTTPAPRPPTPAPTIASQPLSLRPEACRPAAGGAVHTRGLDFACD

SEQ ID NO: 19 - protein, 24 aa, synthetic construct
IYIWAPLAGTCGVLLLSLVITLYC

SEQ ID NO: 20 - protein, 42 aa, synthetic construct
KRGRKKLLYIFKQPFMRPVQTTQEEDGCSCRFPEEEEGGCEL

SEQ ID NO: 21 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 21 aa, synthetic construct
MALPVTALLLPLALLLHAARP

SEQ ID NO: 23 - protein, 241 aa, synthetic construct
AVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSS

SEQ ID NO: 24 - protein, 242 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 25 - protein, 152 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSGGGGSVAISTSTVLLCGLSAVSLLACYL

SEQ ID NO: 26 - protein, 487 aa, synthetic construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 488 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 22 aa, synthetic construct
GSGATNFSLLKQAGDVEENPGP

SEQ ID NO: 29 - protein, 671 aa, synthetic construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 30 - protein, 672 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 31 - protein, 671 aa, synthetic construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDLTLYTESDVHPSCKVTAMKCFLLELQVISLESGDASIHDTVENLIILANNSLSSNGNVTESGCKECEELEEKNIKEFLQSFVHIVQMFINTS

SEQ ID NO: 32 - protein, 671 aa, synthetic construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATEYTESDVHPSCKVTAMKCFLLELQVISLESGDASIHDTVENLIILANNSLSSNGNVTESGCKECEELEEKNIKEFLQSFVHIVQMFINTS

SEQ ID NO: 33 - protein, 671 aa, synthetic construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLGTESDVHPSCKVTAMKCFLLELQVISLESGDASIHDTVENLIILANNSLSSNGNVTESGCKECEELEEKNIKEFLQSFVHIVQMFINTS

SEQ ID NO: 34 - protein, 671 aa, synthetic construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISELKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 35 - protein, 671 aa, synthetic construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHETVENLIILANNSLSSNGNVTESGCKECEELEEKNIKEFLQSFVHIVQMFINTS

SEQ ID NO: 36 - protein, 671 aa, synthetic construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDGVENLIILANNSLSSNGNVTESGCKECEELEEKNIKEFLQSFVHIVQMFINTS

SEQ ID NO: 37 - protein, 671 aa, synthetic construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDIVENLIILANNSLSSNGNVTESGCKECEELEEKNIKEFLQSFVHIVQMFINTS

SEQ ID NO: 38 - protein, 671 aa, synthetic construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECKELEEKNIKEFLQSFVHIVQMFINTS

SEQ ID NO: 39 - protein, 681 aa, synthetic construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMYRMQLLSCIALSLALVTNSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSGGGGSVAISTSTVLLCGLSAVSLLACYL

SEQ ID NO: 40 - protein, 20 aa, synthetic construct
GGGGSGGGGSGGGGSGGGGS

SEQ ID NO: 41 - protein, 5 aa, synthetic construct
GGGGS

SEQ ID NO: 42 - protein, 671 aa, synthetic construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDADIHDTVENLIILANNSLSSNGNVTESGCKECEELEEKNIKEFLQSFVHIVQMFINTS

SEQ ID NO: 43 - protein, 671 aa, synthetic construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDQVENLIILANNSLSSNGNVTESGCKECEELEEKNIKEFLQSFVHIVQMFINTS

SEQ ID NO: 44 - protein, 671 aa, synthetic construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDVVENLIILANNSLSSNGNVTESGCKECEELEEKNIKEFLQSFVHIVQMFINTS

SEQ ID NO: 45 - protein, 671 aa, synthetic construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDPVENLIILANNSLSSNGNVTESGCKECEELEEKNIKEFLQSFVHIVQMFINTS

SEQ ID NO: 46 - protein, 671 aa, synthetic construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDLVENLIILANNSLSSNGNVTESGCKECEELEEKNIKEFLQSFVHIVQMFINTS

SEQ ID NO: 47 - protein, 671 aa, synthetic construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDAVENLIILANNSLSSNGNVTESGCKECEELEEKNIKEFLQSFVHIVQMFINTS

SEQ ID NO: 48 - protein, 671 aa, synthetic construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDSVENLIILANNSLSSNGNVTESGCKECEELEEKNIKEFLQSFVHIVQMFINTS

SEQ ID NO: 49 - protein, 671 aa, synthetic construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDYVENLIILANNSLSSNGNVTESGCKECEELEEKNIKEFLQSFVHIVQMFINTS

SEQ ID NO: 50 - protein, 175 aa, synthetic construct
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 51 - protein, 65 aa, synthetic construct
ITCPPPMSVEHADIWVKSYSLYSRERYICNSGFKRKAGTSSLTECVLNKATNVAHWTTPSLKCIR

SEQ ID NO: 52 - protein, 525 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 53 - protein, 369 aa, synthetic construct
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 54 - protein, 237 aa, synthetic construct
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 55 - protein, 23 aa, synthetic construct
VAISTSTVLLCGLSAVSLLACYL

SEQ ID NO: 56 - protein, 423 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 57 - protein, 352 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDGVENLIILANNSLSSNGNVTESGCKECEELEEKNIKEFLQSFVHIVQMFINTSGGGGSGGGGSGGGGSITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 58 - protein, 242 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDGVENLIILANNSLSSNGNVTESGCKECEELEEKNIKEFLQSFVHIVQMFINTSGGGGSGGGGSGGGGSITCPPPMSVEHADIWVKSYSLYSRERYICNSGFKRKAGTSSLTECVLNKATNVAHWTTPSLKCIR

SEQ ID NO: 59 - protein, 280 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDGVENLIILANNSLSSNGNVTESGCKECEELEEKNIKEFLQSFVHIVQMFINTSGGGGSGGGGSGGGGSITCPPPMSVEHADIWVKSYSLYSRERYICNSGFKRKAGTSSLTECVLNKATNVAHWTTPSLKCIRGGGGSGGGGSGGGGSVAISTSTVLLCGLSAVSLLACYL

SEQ ID NO: 60 - protein, 1076 aa, synthetic construct
MAAPALSWRLPLLILLLPLATSWASANWVNVISDLKKIEDLIQSMHIDATLYTESDVHPSCKVTAMKCFLLELQVISLESGDASIHDGVENLIILANNSLSSNGNVTESGCKECEELEEKNIKEFLQSFVHIVQMFINTSGGGGSGGGGSGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVGSGATNFSLLKQAGDVEENPGPMLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 61 - protein, 396 aa, synthetic construct
MAPRRARGCRTLGLPALLLLLLLRPPATRGNWVNVISDLKKIEDLIQSMHIDATLYTESDVHPSCKVTAMKCFLLELQVISLESGDASIHDGVENLIILANNSLSSNGNVTESGCKECEELEEKNIKEFLQSFVHIVQMFINTSGGGGSGGGGSGGGGSITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 62 - protein, 680 aa, synthetic construct
MAAPALSWRLPLLILLLPLATSWASANWVNVISDLKKIEDLIQSMHIDATLYTESDVHPSCKVTAMKCFLLELQVISLESGDASIHDGVENLIILANNSLSSNGNVTESGCKECEELEEKNIKEFLQSFVHIVQMFINTSGGGGSGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 63 - protein, 498 aa, synthetic construct
MLKPSLPFTSLLFLQLPLLGVGNWVNVISDLKKIEDLIQSMHIDATLYTESDVHPSCKVTAMKCFLLELQVISLESGDASIHDGVENLIILANNSLSSNGNVTESGCKECEELEEKNIKEFLQSFVHIVQMFINTSGGGGSGGGGSGGGGSLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 64 - protein, 172 aa, synthetic construct
MYRMQLLSCIALSLALVTNSNWVNVISDLKKIEDLIQSMHIDATLYTESDVHPSCKVTAMKCFLLELQVISLESGDASIHDGVENLIILANNSLSSNGNVTESGCKECEELEEKNIKEFLQSFVHIVQMFINTSGGGGSGGGGSGGGGSVAISTSTVLLCGLSAVSLLACYL

SEQ ID NO: 65 - protein, 648 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDGVENLIILANNSLSSNGNVTESGCKECEELEEKNIKEFLQSFVHIVQMFINTSGGGGSGGGGSGGGGSGGGGSAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - protein, 631 aa, synthetic construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSGGGGSGGGGSGGGGSNWVNVISDLKKIEDLIQSMHIDATLYTESDVHPSCKVTAMKCFLLELQVISLESGDASIHDGVENLIILANNSLSSNGNVTESGCKECEELEEKNIKEFLQSFVHIVQMFINTSGGGGSGGGGSGGGGSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - skipped

SEQ ID NO: 68 - protein, 5 aa, synthetic construct
  REPEAT 1..5
GSGGS

SEQ ID NO: 69 - protein, 4 aa, synthetic construct
  REPEAT 1..4
GGGS

SEQ ID NO: 70 - protein, 48 aa, synthetic construct
MRISKPHLRSISIQCYLCLLLNSHFLTEAGIHVFILGCFSAGLPKTEA

SEQ ID NO: 71 - protein, 26 aa, synthetic construct
MAAPALSWRLPLLILLLPLATSWASA

SEQ ID NO: 72 - protein, 30 aa, synthetic construct
MAPRRARGCRTLGLPALLLLLLLRPPATRG

SEQ ID NO: 73 - protein, 22 aa, synthetic construct
MLKPSLPFTSLLFLQLPLLGVG

SEQ ID NO: 74 - protein, 20 aa, synthetic construct
MYRMQLLSCIALSLALVTNS

SEQ ID NO: 75 - protein, 631 aa, synthetic construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSGGGGSGGGGSGGGGSNWVNVISELKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSGGGGSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 1076 aa, synthetic construct
MAAPALSWRLPLLILLLPLATSWASANWVNVISELKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVGSGATNFSLLKQAGDVEENPGPMLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 77 - protein, 414 aa, synthetic construct
MRISKPHLRSISIQCYLCLLLNSHFLTEAGIHVFILGCFSAGLPKTEANWVNVISELKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSGGGGSITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 78 - protein, 114 aa, synthetic construct
NWYNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 79 - protein, 114 aa, synthetic construct
NWVNVISDLKKIEDLIQSMHIDATFYTESDVHPSCKVTAMKCFLLELQVISLESGDASIHDTVENLIILANNSLSSNGNVTESGCKECEELEEKNIKEFLQSFVHIVQMFINTS

SEQ ID NO: 80 - protein, 245 aa, synthetic construct
DIVMTQSPLSLPVTPGEPASISCRSSQTIVHSNGNTYLEWYLQKPGQSPQLLIYKVSNRFSGVPDRFSGSGSGTDFTLKISRVEAEDVGVYYCFQVSHVPYTFGGGTKLEIKGSTSGSGKPGSGEGSTKGQVQLVQSGAEVKKPGASVKVSCKASGYTFTDYEMHWVRQATGQGLEWMGGIDPETGNTAYSQKFKGRVTMTRNTSISTAYMELSSLRSEDTAVYYCARYYSFAYWGQGTLVTVSS

SEQ ID NO: 81 - protein, 490 aa, synthetic construct
MALPVTALLLPLALLLHAARPDIVMTQSPLSLPVTPGEPASISCRSSQTIVHSNGNTYLEWYLQKPGQSPQLLIYKVSNRFSGVPDRFSGSGSGTDFTLKISRVEAEDVGVYYCFQVSHVPYTFGGGTKLEIKGSTSGSGKPGSGEGSTKGQVQLVQSGAEVKKPGASVKVSCKASGYTFTDYEMHWVRQATGQGLEWMGGIDPETGNTAYSQKFKGRVTMTRNTSISTAYMELSSLRSEDTAVYYCARYYSFAYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 82 - protein, 674 aa, synthetic construct
MALPVTALLLPLALLLHAARPDIVMTQSPLSLPVTPGEPASISCRSSQTIVHSNGNTYLEWYLQKPGQSPQLLIYKVSNRFSGVPDRFSGSGSGTDFTLKISRVEAEDVGVYYCFQVSHVPYTFGGGTKLEIKGSTSGSGKPGSGEGSTKGQVQLVQSGAEVKKPGASVKVSCKASGYTFTDYEMHWVRQATGQGLEWMGGIDPETGNTAYSQKFKGRVTMTRNTSISTAYMELSSLRSEDTAVYYCARYYSFAYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 83 - protein, 674 aa, synthetic construct
MALPVTALLLPLALLLHAARPDIVMTQSPLSLPVTPGEPASISCRSSQTIVHSNGNTYLEWYLQKPGQSPQLLIYKVSNRFSGVPDRFSGSGSGTDFTLKISRVEAEDVGVYYCFQVSHVPYTFGGGTKLEIKGSTSGSGKPGSGEGSTKGQVQLVQSGAEVKKPGASVKVSCKASGYTFTDYEMHWVRQATGQGLEWMGGIDPETGNTAYSQKFKGRVTMTRNTSISTAYMELSSLRSEDTAVYYCARYYSFAYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWYNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 84 - protein, 674 aa, synthetic construct
MALPVTALLLPLALLLHAARPDIVMTQSPLSLPVTPGEPASISCRSSQTIVHSNGNTYLEWYLQKPGQSPQLLIYKVSNRFSGVPDRFSGSGSGTDFTLKISRVEAEDVGVYYCFQVSHVPYTFGGGTKLEIKGSTSGSGKPGSGEGSTKGQVQLVQSGAEVKKPGASVKVSCKASGYTFTDYEMHWVRQATGQGLEWMGGIDPETGNTAYSQKFKGRVTMTRNTSISTAYMELSSLRSEDTAVYYCARYYSFAYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATFYTESDVHPSCKVTAMKCFLLELQVISLESGDASIHDTVENLIILANNSLSSNGNVTESGCKECEELEEKNIKEFLQSFVHIVQMFINTS

SEQ ID NO: 85 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR